(19) United States
(12) Patent Application Publication
(10) Pub. No.: US 2022/0259612 A1
(43) Pub. Date: Aug. 18, 2022

(54) METHODS AND COMPOSITIONS FOR MODIFYING CYTOKININ OXIDASE LEVELS IN PLANTS

(71) Applicants: Pairwise Plants Services, Inc., Durtham, NC (US); Monsanto Technology LLC, St. Louis, MO (US)

(72) Inventors: Lolita George Mathew, Cary, NC (US); Shunhong Dai, St. Louis, MO (US); Huachun Larue, St. Louis, MO (US); Benjamin Julius, St. Louis, MO (US); Brent Delbert Brower-Toland, St. Louis, MO (US); Thomas L. Slewinski, St. Louis, MO (US); HaeJin Kim, Durham, NC (US)

(21) Appl. No.: 17/668,570

(22) Filed: Feb. 10, 2022

Related U.S. Application Data
(60) Provisional application No. 63/148,439, filed on Feb. 11, 2021.

(51) Int. Cl. C12N 15/82; C07K 14/415; C12N 9/22; C12N 15/113

(52) U.S. Cl. CPC C12N 15/8261; C07K 14/415; C12N 2310/20; C12N 9/22; C12N 15/113; C12N 15/8213

(57) ABSTRACT

This invention relates to compositions and methods for improving/enhancing yield traits by modifying cytokinin oxidase (CKX) levels in a plant. The invention further relates to plants produced using the methods and compositions of the invention.

### STATEMENT OF PRIORITY

This application claims the benefit, under 35 U.S.C. § 119 (e), of U.S. Provisional Application No. 63/148,439 filed on Feb. 11, 2021, the entire contents of which is incorporated by reference herein.

### STATEMENT REGARDING ELECTRONIC FILING OF A SEQUENCE LISTING

A Sequence Listing in ASCII text format, submitted under 37 C.F.R. § 1.821, entitled 1499.48_ST25.txt, 1,990,037 bytes in size, generated on Feb. 5, 2022 and filed via EFS-Web, is provided in lieu of a paper copy. This Sequence Listing is hereby incorporated herein by reference into the specification for its disclosures.

### FIELD OF THE INVENTION

This invention relates to compositions and methods for improving or enhancing yield traits by modifying cytokinin oxidase (CKX) levels in a plant. The invention further relates to plants produced using the methods and compositions of the invention.

### BACKGROUND OF THE INVENTION

Soybean is a key component of global food security providing high protein animal feed and over half of the world's oilseed production. With a growing population to feed, there is continuous need to increase the crop yields. Currently, key staple crops, including soybean, increase yield only by 0.9-1.6% per year and this magnitude of yield increase is not enough to meet the future needs in food production. The present invention addresses these shortcomings in the art by providing new compositions and methods for improving/enhancing yield traits in plants including soybean.

### SUMMARY OF THE INVENTION

One aspect of the invention provides a plant or plant part thereof comprising at least one non-natural mutation in at least one endogenous Cytokinin Oxidase/Dehydrogenase (CKX) gene encoding a CKX protein.

Another aspect of the invention provides a plant cell comprising an editing system, the editing system comprising (a) a CRISPR-associated effector protein; and (b) a guide nucleic acid (gRNA, gDNA, crRNA, crDNA, sgRNA, sgDNA) comprising a spacer sequence with complementarity to an endogenous target gene encoding a CKX protein in the plant cell.

A further aspect of the invention provides a plant cell comprising at least one non-natural mutation within an endogenous Cytokinin Oxidase/Dehydrogenase (CKX) gene that results in a neo-allele with altered level of expression or a null allele or knockout of the CKX gene, wherein the at least one non-natural mutation is a base substitution, base insertion or a base deletion that is introduced using an editing system that comprises a nucleic acid binding domain that binds to a target site in the CKX gene.

Also provided is a method of providing a plurality of plants having improved yield traits, the method comprising planting two or more plants of the invention in a growing area, thereby providing a plurality of plants having at least one improved yield trait(s) as compared to a plurality of control plants not comprising the at least one non-natural mutation.

The invention further provides a method of producing/breeding a transgene-free genome-edited plant, comprising: (a) crossing a plant of the invention with a transgene free plant, thereby introducing the mutation into the plant that is transgene-free; and (b) selecting a progeny plant that comprises the mutation but is transgene-free, thereby producing a transgene free genome-edited plant.

Another aspect of the invention provides a method for editing a specific site in the genome of a plant cell, the method comprising: cleaving, in a site-specific manner, a target site within an endogenous Cytokinin Oxidase/Dehydrogenase (CKX) gene in the plant cell, wherein the endogenous CKX gene (a) comprises a sequence having at least 80% sequence identity to any one of the nucleotide sequences of SEQ ID NOs:72, 73, 75, 76, 78, 79, 81, 82, 84, 87, 88, or 91; (b) comprises a region having at least 80% sequence identity to any one of the nucleotide sequences of SEQ ID NOs:93-98; and/or (c) encodes a polypeptide having at least 80% sequence identity to any one of the amino acid sequences of SEQ ID NOs:74, 77, 80, 83, 89, or 92, thereby generating an edit in the endogenous CKX gene of the plant cell and producing a plant cell comprising an edit in the endogenous CKX gene.

An additional aspect of the invention provides a method for making a plant, comprising: (a) contacting a population of plant cells comprising at least one endogenous Cytokinin Oxidase/Dehydrogenase (CKX) gene with a nuclease targeted to the endogenous CKX gene, wherein the nuclease is linked to a nucleic acid binding domain (e.g., DNA binding domain) (e.g., an editing system) that binds to a target site in the at least one endogenous CKX gene, wherein the at least one endogenous CKX gene (i) comprises a sequence having at least 80% sequence identity to any one of the nucleotide sequences of SEQ ID NOs:72, 73, 75, 76, 78, 79, 81, 82, 84, 87, 88, or 91; (ii) comprises a region having at least 80% sequence identity to any one of the nucleotide sequences of SEQ ID NOs:93-98; and/or (iii) encodes a polypeptide having at least 80% identity to any one of the amino acid sequences of SEQ ID NOs: 74, 77, 80, 83, 89, or 92; (b) selecting from the population a plant cell that comprises a mutation in the at least one endogenous CKX gene, wherein the mutation is a substitution and/or a deletion; and (c) growing the selected plant cell into a plant comprising the mutation in the at least one endogenous CKX gene.

In an additional aspect, a method improving yield traits in a plant or part thereof, comprising (a) contacting a plant cell comprising an endogenous Cytokinin Oxidase/Dehydrogenase (CKX) gene with a nuclease targeting the endogenous CKX gene, wherein the nuclease is linked to a nucleic acid binding domain (e.g., a DNA binding domain) that binds to a target site in the endogenous CKX gene, wherein the endogenous CKX gene: (i) comprises a sequence having at least 80% sequence identity to any one of the nucleotide sequences of SEQ ID NOs:72, 73, 75, 76, 78, 79, 81, 82, 84, 87, 88, or 91; (ii) comprises a region having at least 80% sequence identity to any one of the nucleotide sequences of SEQ ID NOs:93-98; and/or (iii) encodes a polypeptide having at least 80% identity to any one of the amino acid sequences of SEQ ID NOs: 74, 77, 80, 83, 89, or 92; and (b) growing the plant cell into a plant comprising the mutation in the endogenous CKX gene, thereby improving yield traits (e.g., increased seed number, increased seed size; increased pod number; increased yield or improved yield traits under increased planting density) in the plant or part thereof.

In another aspect, a method is provided for producing a plant or part thereof comprising at least one cell having a mutation in an endogenous Cytokinin Oxidase/Dehydrogenase (CKX) gene, the method comprising contacting a target site in the endogenous CKX gene in the plant or plant part with a nuclease comprising a cleavage domain and a nucleic acid binding domain (e.g., a DNA binding domain), wherein the nucleic acid binding domain of the nuclease binds to a target site in the endogenous CKX gene, the endogenous CKX gene: (a) comprising a sequence having at least 80% sequence identity to any one of the nucleotide sequences of SEQ ID NOs:72, 73, 75, 76, 78, 79, 81, 82, 84, 87, 88, or 91; (b) comprising a region having at least 80% sequence identity to any one of the nucleotide sequences of SEQ ID NOs:93-98; and/or (c) encoding a polypeptide having at least 80% identity to any one of the amino acid sequences of SEQ ID NOs: 74, 77, 80, 83, 89, or 92, thereby producing the plant or part thereof comprising at least one cell having a mutation in the endogenous CKX gene.

In a further aspect, a method of producing a plant or part thereof comprising a mutation in an endogenous Cytokinin Oxidase/Dehydrogenase (CKX) gene and improved yield traits, the method comprising contacting a target site in an endogenous CKX gene in the plant or plant part with a nuclease comprising a cleavage domain and a nucleic acid binding domain (e.g., a DNA binding domain), wherein the nucleic acid binding domain binds to a target site in the endogenous CKX gene, the endogenous CKX gene: (a) comprising a sequence having at least 80% sequence identity to any one of the nucleotide sequences of SEQ ID NOs:72, 73, 75, 76, 78, 79, 81, 82, 84, 87, 88, or 91; (b) comprising a region having at least 80% sequence identity to any one of the nucleotide sequences of SEQ ID NOs:93-98; and/or (c) encoding a polypeptide having at least 80% identity to any one of the amino acid sequences of SEQ ID NOs: 74, 77, 80, 83, 89, or 92, thereby producing the plant or part thereof comprising a mutation in the endogenous CKX gene and exhibiting improved yield traits.

An additional aspect of the invention provides a guide nucleic acid that that binds to a target site in a Cytokinin Oxidase/Dehydrogenase (CKX) gene, the CKX gene: (a) comprising a sequence having at least 80% sequence identity to any one of the nucleotide sequences of SEQ ID NOs:72, 73, 75, 76, 78, 79, 81, 82, 84, 87, 88, or 91; (b) comprising a region having at least 80% sequence identity to any one of the nucleotide sequences of SEQ ID NOs:93-98; and/or (c) encoding a polypeptide having at least 80% identity to any one of the amino acid sequences of SEQ ID NOs: 74, 77, 80, 83, 89, or 92.

A further aspect of the invention provides a system comprising a guide nucleic acid of the invention and a CRISPR-Cas effector protein that associates with the guide nucleic acid.

Another aspect of the invention provides gene editing system comprising a CRISPR-Cas effector protein in association with a guide nucleic acid, wherein the guide nucleic acid comprises a spacer sequence that is complementary to and binds to a Cytokinin Oxidase/Dehydrogenase (CKX) gene.

An additional aspect of the invention provides a complex comprising a CRISPR-Cas effector protein comprising a cleavage domain and a guide nucleic acid, wherein the guide nucleic acid binds to a target site in an endogenous Cytokinin Oxidase/Dehydrogenase (CKX) gene, wherein the CKX gene: (a) comprises a sequence having at least 80% sequence identity to any one of the nucleotide sequences of SEQ ID NOs:72, 73, 75, 76, 78, 79, 81, 82, 84, 87, 88, or 91; (b) comprises a region having at least 80% sequence identity to any one of the nucleotide sequences of SEQ ID NOs:93-98; and/or (c) encodes a polypeptide having at least 80% sequence identity to any one of the amino acid sequences of SEQ ID NOs:74, 77, 80, 83, 89, or 92, wherein the cleavage domain cleaves a target strand in the CKX gene.

An further aspect provides an expression cassette comprising ((a) a polynucleotide encoding CRISPR-Cas effector protein comprising a cleavage domain and (b) a guide nucleic acid that binds to a target site in an endogenous Cytokinin Oxidase/Dehydrogenase (CKX) gene, wherein the guide nucleic acid comprises a spacer sequence that is complementary to and binds to a portion of the endogenous CKX gene, the endogenous CKX gene having at least 80% sequence identity to any one of the nucleotide sequences of SEQ ID NOs:72, 73, 75, 76, 78, 79, 81, 82, 84, 87, 88, or 91 or encoding a sequence having at least 80% sequence identity to any one of the amino acid sequences of SEQ ID NOs:74, 77, 80, 83, 89, or 92, optionally wherein the spacer sequence is complementary to and binds to a portion of the endogenous CKX gene having at least 80% sequence identity to any one of the nucleotide sequences of SEQ ID NOs:93-98.

Another aspect of the invention provides a nucleic acid comprising a mutated Cytokinin Oxidase/Dehydrogenase (CKX) gene, wherein the mutated CKX gene produces a truncated CKX protein or no protein.

Further provided are plants comprising in their genome one or more Cytokinin Oxidase/Dehydrogenase (CKX) genes having a non-natural mutation produced by the methods of the invention as well as polypeptides, polynucleotides, nucleic acid constructs, expression cassettes and vectors for making a plant of this invention.

These and other aspects of the invention are set forth in more detail in the description of the invention below.

### BRIEF DESCRIPTION OF THE SEQUENCES

SEQ ID NOs:1-17 are exemplary Cas12a amino acid sequences useful with this invention.

SEQ ID NOs:18-20 are exemplary Cas12a nucleotide sequences useful with this invention.

SEQ ID NO:21-22 are exemplary regulatory sequences encoding a promoter and intron.

SEQ ID NOs:23-29 are exemplary cytosine deaminase sequences useful with this invention.

SEQ ID NOs:30-40 are exemplary adenine deaminase amino acid sequences useful with this invention.

SEQ ID NO:41 is an exemplary uracil-DNA glycosylase inhibitor (UGI) sequences useful with this invention.

SEQ ID NOs:42-44 provides an example of a protospacer adjacent motif position for a Type V CRISPR-Cas12a nuclease.

SEQ ID NOs:45-47 provide example peptide tags and affinity polypeptides useful with this invention.

SEQ ID NOs:48-58 provide example RNA recruiting motifs and corresponding affinity polypeptides useful with this invention.

SEQ ID NOs:59-60 are example Cas9 polypeptide sequences useful with this invention.

SEQ ID NOs:61-71 are example Cas9 polynucleotide sequences useful with this invention.

SEQ ID NOs: 72, 75, 78, 81, 84, 87, or 90 are example CKX genomic sequences (CKX1, CKX2, CKX3, CKX4, CKX5, CKX6, and CKX5, respectively).

SEQ ID NOs:73, 76, 79, 82, 85, 88 or 91 are example CKX coding (cds) sequences (CKX1, CKX2, CKX3, CKX4, CKX5, CKX6, and CKX5, respectively).

SEQ ID NOs:74, 77, 80, 83, 86, 89, or 92 are example CKX polypeptide sequences (CKX1, CKX2, CKX3, CKX4, CKX5, CKX6, and CKX5, respectively).

SEQ ID NOs:92-98 are example nucleic acid sequences (regions) from CKX polynucleotides (example regions (e.g., example target sites) from CKX1, CKX2, CKX3, CKX4, CKX5, CKX6, and CKX5, respectively).

SEQ ID NOs:99-101 are example spacer sequences for a CKX1 gene.

SEQ ID NOs:102-104 are example spacer sequences for a CKX2 gene.

SEQ ID NOs:105-107 are example spacer sequences for a CKX3 gene.

SEQ ID NO:108 and SEQ ID NO:109 are example spacer sequences for a CKX4 gene.

SEQ ID NO:110 and SEQ ID NO:111 are example spacer sequences for a CKX5 gene.

SEQ ID NO:112 and SEQ ID NO:113 are example spacer sequences for a CKX6 gene.

SEQ ID NOs:114-284 are example edited sequences.

### DETAILED DESCRIPTION

The present invention now will be described hereinafter with reference to the examples, in which embodiments of the invention are shown. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

As used in the description of the invention and the appended claims, the singular forms “a,” “an” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount or concentration and the like, is meant to encompass variations of ±10%, ±5%, ±1%, ±0.5%, or even ±0.1% of the specified value as well as the specified value. For example, “about X” where X is the measurable value, is meant to include X as well as variations of ±10%, ±5%, ±1%, ±0.5%, or even ±0.1% of X. A range provided herein for a measurable value may include any other range and/or individual value therein.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

Recitation of ranges of values herein are merely intended to serve as a shorthand method of referring individually to each separate value falling within the range, unless otherwise indicated herein, and each separate value is incorporated into the specification as if it were individually recited herein. For example, if the range 10 to 15 is disclosed, then 11, 12, 13, and 14 are also disclosed.

The term “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

As used herein, the terms “increase,” “increasing,” “increased,” “enhance,” “enhanced,” “enhancing,” and “enhancement” (and grammatical variations thereof) describe an elevation of at least about 15%, 20%, 25%, 50%, 75%, 100%, 150%, 200%, 300%, 400%, 500% or more as compared to a control.

As used herein, the terms “reduce,” “reduced,” “reducing,” “reduction,” “diminish,” and “decrease” (and grammatical variations thereof), describe, for example, a decrease of at least about 5%, 10%, 15%, 20%, 25%, 35%, 50%, 75%, 80%, 85%, 90%, 95%, 96%, 97%, 98%, 99%, 99.5%, 99.6%, 99.7%, 99.8%, 99.9%, or 100% as compared to a control. In some embodiments, the reduction can result in no or essentially no (i.e., an insignificant amount, e.g., less than about 10% or even 5%) detectable activity or amount.

As used herein, the terms “express,” “expresses,” “expressed” or “expression,” and the like, with respect to a nucleic acid molecule and/or a nucleotide sequence (e.g., RNA or DNA) indicates that the nucleic acid molecule and/or a nucleotide sequence is transcribed and, optionally, translated. Thus, a nucleic acid molecule and/or a nucleotide sequence may express a polypeptide of interest or, for example, a functional untranslated RNA.

A “heterologous” or a “recombinant” nucleotide sequence is a nucleotide sequence not naturally associated with a host cell into which it is introduced, including non-naturally occurring multiple copies of a naturally occurring nucleotide sequence.

A “native” or “wild type” nucleic acid, nucleotide sequence, polypeptide or amino acid sequence refers to a naturally occurring or endogenous nucleic acid, nucleotide sequence, polypeptide or amino acid sequence. Thus, for example, a “wild type mRNA” is an mRNA that is naturally occurring in or endogenous to the reference organism.

As used herein, the term “heterozygous” refers to a genetic status wherein different alleles reside at corresponding loci on homologous chromosomes.

As used herein, the term “homozygous” refers to a genetic status wherein identical alleles reside at corresponding loci on homologous chromosomes.

As used herein, the term “allele” refers to one of two or more different nucleotides or nucleotide sequences that occur at a specific locus.

A “null allele” is a nonfunctional allele caused by a genetic mutation that results in a complete lack of production of the corresponding protein or produces a protein that is non-functional.

A “recessive mutation” is a mutation in a gene that produces a phenotype when homozygous but the phenotype is not observable when the locus is heterozygous.

A “dominant negative mutation” is a mutation that produces an altered gene product (e.g., having an aberrant function relative to wild type), which gene product adversely affects the function of the wild-type allele or gene product. For example, a “dominant negative mutation” may block a function of the wild type gene product. A dominant negative mutation may also be referred to as an “antimorphic mutation.”

A “semi-dominant mutation” refers to a mutation in which the penetrance of the phenotype in a heterozygous organism is less than that observed for a homozygous organism.

A “weak loss-of-function mutation” is a mutation that results in a gene product having partial function or reduced function (partially inactivated) as compared to the wild type gene product.

A “hypomorphic mutation” is a mutation that results in a partial loss of gene function, which may occur through reduced expression (e.g., reduced protein and/or reduced RNA) or reduced functional performance (e.g., reduced activity), but not a complete loss of function/activity. A “hypomorphic” allele is a semi-functional allele caused by a genetic mutation that results in production of the corresponding protein that functions at anywhere between 1% and 99% of normal efficiency.

A “hypermorphic mutation” is a mutation that results in increased expression of the gene product and/or increased activity of the gene product.

A “locus” is a position on a chromosome where a gene or marker or allele is located. In some embodiments, a locus may encompass one or more nucleotides.

As used herein, the terms “desired allele,” “target allele” and/or “allele of interest” are used interchangeably to refer to an allele associated with a desired trait. In some embodiments, a desired allele may be associated with either an increase or a decrease (relative to a control) of or in a given trait, depending on the nature of the desired phenotype.—In some embodiments of this invention, the phrase “desired allele,” “target allele” or “allele of interest” refers to an allele(s) that is associated with increased yield under non-water stress conditions in a plant relative to a control plant not having the target allele or alleles.

A marker is “associated with” a trait when said trait is linked to it and when the presence of the marker is an indicator of whether and/or to what extent the desired trait or trait form will occur in a plant/germplasm comprising the marker. Similarly, a marker is “associated with” an allele or chromosome interval when it is linked to it and when the presence of the marker is an indicator of whether the allele or chromosome interval is present in a plant/germplasm comprising the marker.

As used herein, the terms “backcross” and “backcrossing” refer to the process whereby a progeny plant is crossed back to one of its parents one or more times (e.g., 1, 2, 3, 4, 5, 6, 7, 8, etc.). In a backcrossing scheme, the “donor” parent refers to the parental plant with the desired gene or locus to be introgressed. The “recipient” parent (used one or more times) or “recurrent” parent (used two or more times) refers to the parental plant into which the gene or locus is being introgressed. For example, see Ragot, M. et al. Marker-assisted Backcrossing: A Practical Example, in TECHNIQUES ET UTILISATIONS DES MARQUEURS MOLECULAIRES LES COLLOQUES, Vol. 72, pp. 45-56 (1995); and Openshaw et al., Marker-assisted Selection in Backcross Breeding, in PROCEEDINGS OF THE SYMPOSIUM “ANALYSIS OF MOLECULAR MARKER DATA,” pp. 41-43 (1994). The initial cross gives rise to the F1 generation. The term “BC1” refers to the second use of the recurrent parent, “BC2” refers to the third use of the recurrent parent, and so on.

As used herein, the terms “cross” or “crossed” refer to the fusion of gametes via pollination to produce progeny (e.g., cells, seeds or plants). The term encompasses both sexual crosses (the pollination of one plant by another) and selfing (self-pollination, e.g., when the pollen and ovule are from the same plant). The term “crossing” refers to the act of fusing gametes via pollination to produce progeny.

As used herein, the terms “introgression,” “introgressing” and “introgressed” refer to both the natural and artificial transmission of a desired allele or combination of desired alleles of a genetic locus or genetic loci from one genetic background to another. For example, a desired allele at a specified locus can be transmitted to at least one progeny via a sexual cross between two parents of the same species, where at least one of the parents has the desired allele in its genome. Alternatively, for example, transmission of an allele can occur by recombination between two donor genomes, e.g., in a fused protoplast, where at least one of the donor protoplasts has the desired allele in its genome. The desired allele may be a selected allele of a marker, a QTL, a transgene, or the like. Offspring comprising the desired allele can be backcrossed one or more times (e.g., 1, 2, 3, 4, or more times) to a line having a desired genetic background, selecting for the desired allele, with the result being that the desired allele becomes fixed in the desired genetic background. For example, a marker associated with increased yield under non-water stress conditions may be introgressed from a donor into a recurrent parent that does not comprise the marker and does not exhibit increased yield under non-water stress conditions. The resulting offspring could then be backcrossed one or more times and selected until the progeny possess the genetic marker(s) associated with increased yield under non-water stress conditions in the recurrent parent background.

A “genetic map” is a description of genetic linkage relationships among loci on one or more chromosomes within a given species, generally depicted in a diagrammatic or tabular form. For each genetic map, distances between loci are measured by the recombination frequencies between them. Recombination between loci can be detected using a variety of markers. A genetic map is a product of the mapping population, types of markers used, and the polymorphic potential of each marker between different populations. The order and genetic distances between loci can differ from one genetic map to another.

As used herein, the term “genotype” refers to the genetic constitution of an individual (or group of individuals) at one or more genetic loci, as contrasted with the observable and/or detectable and/or manifested trait (the phenotype). Genotype is defined by the allele(s) of one or more known loci that the individual has inherited from its parents. The term genotype can be used to refer to an individual's genetic constitution at a single locus, at multiple loci, or more generally, the term genotype can be used to refer to an individual's genetic make-up for all the genes in its genome. Genotypes can be indirectly characterized, e.g., using markers and/or directly characterized by nucleic acid sequencing.

As used herein, the term “germplasm” refers to genetic material of or from an individual (e.g., a plant), a group of individuals (e.g., a plant line, variety or family), or a clone derived from a line, variety, species, or culture. The germplasm can be part of an organism or cell or can be separate from the organism or cell. In general, germplasm provides genetic material with a specific genetic makeup that provides a foundation for some or all of the hereditary qualities of an organism or cell culture. As used herein, germplasm includes cells, seed or tissues from which new plants may be grown, as well as plant parts that can be cultured into a whole plant (e.g., leaves, stems, buds, roots, pollen, cells, etc.).

As used herein, the terms “cultivar” and “variety” refer to a group of similar plants that by structural or genetic features and/or performance can be distinguished from other varieties within the same species.

As used herein, the terms “exotic,” “exotic line” and “exotic germplasm” refer to any plant, line or germplasm that is not elite. In general, exotic plants/germplasms are not derived from any known elite plant or germplasm, but rather are selected to introduce one or more desired genetic elements into a breeding program (e.g., to introduce novel alleles into a breeding program).

As used herein, the term “hybrid” in the context of plant breeding refers to a plant that is the offspring of genetically dissimilar parents produced by crossing plants of different lines or breeds or species, including but not limited to the cross between two inbred lines.

As used herein, the term “inbred” refers to a substantially homozygous plant or variety. The term may refer to a plant or plant variety that is substantially homozygous throughout the entire genome or that is substantially homozygous with respect to a portion of the genome that is of particular interest.

A “haplotype” is the genotype of an individual at a plurality of genetic loci, i.e., a combination of alleles. Typically, the genetic loci that define a haplotype are physically and genetically linked, i.e., on the same chromosome segment. The term “haplotype” can refer to polymorphisms at a particular locus, such as a single marker locus, or polymorphisms at multiple loci along a chromosomal segment.

As used herein, the term “heterologous” refers to a nucleotide/polypeptide that originates from a foreign species, or, if from the same species, is substantially modified from its native form in composition and/or genomic locus by deliberate human intervention.

A plant in which the activity of at least one CKX polypeptide is modified as described herein may have improved yield traits as compared to a plant that does not comprise the modification (e.g., an increase or a decrease) in CKX activity. As used herein, “improved yield traits” refers to any plant trait associated with growth, for example, biomass, yield, nitrogen use efficiency (NUE), inflorescence size/weight, fruit yield, fruit quality, fruit size, seed size, seed number, foliar tissue weight, nodulation number, nodulation mass, nodulation activity, number of seed heads, number of tillers, number of branches, number of flowers, number of tubers, tuber mass, bulb mass, number of seeds, total seed mass, rate of leaf emergence, rate of tiller/branch emergence, rate of seedling emergence, length of roots, number of roots, size and/or weight of root mass, or any combination thereof. Thus, in some aspects, “improved yield traits” may include, but is not limited to, increased inflorescence production, increased fruit production (e.g., increased number, weight and/or size of fruit; e.g., increase number, weight, and/or size of ears for, e.g., maize), increased fruit quality, increased number, size and/or weight of roots, increased meristem size, increased seed size, increased biomass, increased leaf size, increased nitrogen use efficiency, increased height, increased internode number and/or increased internode length as compared to a control plant or part thereof (e.g., a plant that does not comprise a mutated endogenous CKX nucleic acid (e.g., a mutated CKX1 gene, a mutated CKX2 gene, a mutated CKX3 gene, a mutated CKX4 gene, a mutated CKX5 gene, and/or a mutated CKX6 gene)). Improved yield traits can also result from increased planting density of plants of the invention. Thus, in some aspects, a plant of the invention is capable of being planted at an increased density (as a consequence of altered plant architecture resulting from the endogenous mutation), which results in improved yield traits as compared to a control plant that is planted at the same density. In some aspects, improved yield traits can be expressed as quantity of grain produced per area of land (e.g., bushels per acre of land).

As used herein a “control plant” means a plant that does not contain an edited CKX gene or genes as described herein that imparts an enhanced/improved trait (e.g., yield trait) or altered phenotype. A control plant is used to identify and select a plant edited as described herein and that has an enhanced trait or altered phenotype. A suitable control plant can be a plant of the parental line used to generate a plant comprising a mutated CKX gene(s), for example, a wild type plant devoid of an edit in an endogenous CKX gene as described herein. A suitable control plant can also be a plant that contains recombinant nucleic acids that impart other traits, for example, a transgenic plant having enhanced herbicide tolerance. A suitable control plant can in some cases be a progeny of a heterozygous or hemizygous transgenic plant line that is devoid of a mutated CKX gene as described herein, known as a negative segregant, or a negative isogenic line.

An enhanced trait may be, for example, decreased days from planting to maturity, increased stalk size, increased number of leaves, increased plant height growth rate in vegetative stage, increased ear size, increased ear dry weight per plant, increased number of kernels per ear, increased weight per kernel, increased number of kernels per plant, decreased ear void, extended grain fill period, reduced plant height, increased number of root branches, increased total root length, increased yield, increased nitrogen use efficiency, and increased water use efficiency as compared to a control plant. An altered phenotype may be, for example, plant height, biomass, canopy area, anthocyanin content, chlorophyll content, water applied, water content, and water use efficiency.

As used herein a “trait” is a physiological, morphological, biochemical, or physical characteristic of a plant or particular plant material or cell. In some instances, this characteristic is visible to the human eye and can be measured mechanically, such as seed or plant size, weight, shape, form, length, height, growth rate and development stage, or can be measured by biochemical techniques, such as detecting the protein, starch, certain metabolites, or oil content of seed or leaves, or by observation of a metabolic or physiological process, for example, by measuring tolerance to water deprivation or particular salt or sugar concentrations, or by the measurement of the expression level of a gene or genes, for example, by employing Northern analysis, RT-PCR, microarray gene expression assays, or reporter gene expression systems, or by agricultural observations such as hyperosmotic stress tolerance or yield. Any technique can be used to measure the amount of, comparative level of, or difference in any selected chemical compound or macromolecule in the transgenic plants, however.

As used herein an “enhanced trait” means a characteristic of a plant resulting from mutations in a CKX gene(s) as described herein. Such traits include, but are not limited to, an enhanced agronomic trait characterized by enhanced plant morphology, physiology, growth and development, yield, nutritional enhancement, disease or pest resistance, or environmental or chemical tolerance. In some embodiments, an enhanced trait/altered phenotype may be, for example, decreased days from planting to maturity, increased stalk size, increased number of leaves, increased plant height growth rate in vegetative stage, increased ear size, increased ear dry weight per plant, increased number of kernels per ear, increased weight per kernel, increased number of kernels per plant, decreased ear void, extended grain fill period, reduced plant height, increased number of root branches, increased total root length, drought tolerance, increased water use efficiency, cold tolerance, increased nitrogen use efficiency, and increased yield. In some embodiments, a trait is increased yield under nonstress conditions or increased yield under environmental stress conditions. Stress conditions can include both biotic and abiotic stress, for example, drought, shade, fungal disease, viral disease, bacterial disease, insect infestation, nematode infestation, cold temperature exposure, heat exposure, osmotic stress, reduced nitrogen nutrient availability, reduced phosphorus nutrient availability and high plant density. “Yield” can be affected by many properties including without limitation, plant height, plant biomass, pod number, pod position on the plant, number of internodes, incidence of pod shatter, grain size, ear size, ear tip filling, kernel abortion, efficiency of nodulation and nitrogen fixation, efficiency of nutrient assimilation, resistance to biotic and abiotic stress, carbon assimilation, plant architecture, resistance to lodging, percent seed germination, seedling vigor, and juvenile traits. Yield can also be affected by efficiency of germination (including germination in stressed conditions), growth rate (including growth rate in stressed conditions), flowering time and duration, ear number, ear size, ear weight, seed number per ear or pod, seed size, composition of seed (starch, oil, protein) and characteristics of seed fill.

Also used herein, the term “trait modification” encompasses altering the naturally occurring trait by producing a detectable difference in a characteristic in a plant comprising a mutation in an endogenous CKX gene as described herein relative to a plant not comprising the mutation, such as a wild-type plant, or a negative segregant. In some cases, the trait modification can be evaluated quantitatively. For example, the trait modification can entail an increase or decrease in an observed trait characteristics or phenotype as compared to a control plant. It is known that there can be natural variations in a modified trait. Therefore, the trait modification observed entails a change of the normal distribution and magnitude of the trait characteristics or phenotype in the plants as compared to a control plant.

The present disclosure relates to a plant with improved economically important characteristics, more specifically increased yield. More specifically the present disclosure relates to a plant comprising a mutation(s) in a CKX gene(s) as described herein, wherein the plant has increased yield as compared to a control plant devoid of said mutation(s). In some embodiments, plants produced as described herein exhibit increased yield or improved yield trait components as compared to a control plant. In some embodiments, a plant of the present disclosure exhibits an improved trait that is related to yield, including but not limited to increased nitrogen use efficiency, increased nitrogen stress tolerance, increased water use efficiency and increased drought tolerance, as defined and discussed infra.

Yield can be defined as the measurable produce of economic value from a crop. Yield can be defined in the scope of quantity and/or quality. Yield can be directly dependent on several factors, for example, the number and size of organs, plant architecture (such as the number of branches, plant biomass, etc.), flowering time and duration, grain fill period. Root architecture and development, photosynthetic efficiency, nutrient uptake, stress tolerance, early vigor, delayed senescence and functional stay green phenotypes may be factors in determining yield. Optimizing the above-mentioned factors can therefore contribute to increasing crop yield.

Reference herein to an increase/improvement in yield-related traits can also be taken to mean an increase in biomass (weight) of one or more parts of a plant, which can include above ground and/or below ground (harvestable) plant parts. In particular, such harvestable parts are seeds, and performance of the methods of the disclosure results in plants with increased yield and in particular increased seed yield relative to the seed yield of suitable control plants. The term “yield” of a plant can relate to vegetative biomass (root and/or shoot biomass), to reproductive organs, and/or to propagules (such as seeds) of that plant.

Increased yield of a plant of the present disclosure can be measured in a number of ways, including test weight, seed number per plant, seed weight, seed number per unit area (for example, seeds, or weight of seeds, per acre), bushels per acre, tons per acre, or kilo per hectare. Increased yield can result from improved utilization of key biochemical compounds, such as nitrogen, phosphorous and carbohydrate, or from improved responses to environmental stresses, such as cold, heat, drought, salt, shade, high plant density, and attack by pests or pathogens.

“Increased yield” can manifest as one or more of the following: (i) increased plant biomass (weight) of one or more parts of a plant, particularly aboveground (harvestable) parts, of a plant, increased root biomass (increased number of roots, increased root thickness, increased root length) or increased biomass of any other harvestable part; or (ii) increased early vigor, defined herein as an improved seedling aboveground area approximately three weeks post-germination.

“Early vigor” refers to active healthy plant growth especially during early stages of plant growth, and can result from increased plant fitness due to, for example, the plants being better adapted to their environment (for example, optimizing the use of energy resources, uptake of nutrients and partitioning carbon allocation between shoot and root). Early vigor, for example, can be a combination of the ability of seeds to germinate and emerge after planting and the ability of the young plants to grow and develop after emergence. Plants having early vigor also show increased seedling survival and better establishment of the crop, which often results in highly uniform fields with the majority of the plants reaching the various stages of development at substantially the same time, which often results in increased yield. Therefore, early vigor can be determined by measuring various factors, such as kernel weight, percentage germination, percentage emergence, seedling growth, seedling height, root length, root and shoot biomass, canopy size and color and others.

Further, increased yield can also manifest as increased total seed yield, which may result from one or more of an increase in seed biomass (seed weight) due to an increase in the seed weight on a per plant and/or on an individual seed basis an increased number of, for example, flowers/panicles per plant; an increased number of pods; an increased number of nodes; an increased number of flowers (“florets”) per panicle/plant; increased seed fill rate; an increased number of filled seeds; increased seed size (length, width, area, perimeter), which can also influence the composition of seeds; and/or increased seed volume, which can also influence the composition of seeds. In one embodiment, increased yield can be increased seed yield, for example, increased seed weight; increased number of filled seeds; and increased harvest index.

Increased yield can also result in modified architecture, or can occur because of modified plant architecture.

Increased yield can also manifest as increased harvest index, which is expressed as a ratio of the yield of harvestable parts, such as seeds, over the total biomass

The disclosure also extends to harvestable parts of a plant such as, but not limited to, seeds, leaves, fruits, flowers, bolls, pods, siliques, nuts, stems, rhizomes, tubers and bulbs. The disclosure furthermore relates to products derived from a harvestable part of such a plant, such as dry pellets, powders, oil, fat and fatty acids, starch or proteins.

The present disclosure provides a method for increasing “yield” of a plant or “broad acre yield” of a plant or plant part defined as the harvestable plant parts per unit area, for example seeds, or weight of seeds, per acre, pounds per acre, bushels per acre, tones per acre, tons per acre, kilo per hectare.

As used herein “nitrogen use efficiency” refers to the processes which lead to an increase in the plant's yield, biomass, vigor, and growth rate per nitrogen unit applied. The processes can include the uptake, assimilation, accumulation, signaling, sensing, retranslocation (within the plant) and use of nitrogen by the plant.

As used herein “increased nitrogen use efficiency” refers to the ability of plants to grow, develop, or yield faster or better than normal when subjected to the same amount of available/applied nitrogen as under normal or standard conditions; ability of plants to grow, develop, or yield normally, or grow, develop, or yield faster or better when subjected to less than optimal amounts of available/applied nitrogen, or under nitrogen limiting conditions.

As used herein “nitrogen limiting conditions” refers to growth conditions or environments that provide less than optimal amounts of nitrogen needed for adequate or successful plant metabolism, growth, reproductive success and/or viability.

As used herein the “increased nitrogen stress tolerance” refers to the ability of plants to grow, develop, or yield normally, or grow, develop, or yield faster or better when subjected to less than optimal amounts of available/applied nitrogen, or under nitrogen limiting conditions.

Increased plant nitrogen use efficiency can be translated in the field into either harvesting similar quantities of yield, while supplying less nitrogen, or increased yield gained by supplying optimal/sufficient amounts of nitrogen. The increased nitrogen use efficiency can improve plant nitrogen stress tolerance and can also improve crop quality and biochemical constituents of the seed such as protein yield and oil yield. The terms “increased nitrogen use efficiency”, “enhanced nitrogen use efficiency”, and “nitrogen stress tolerance” are used inter-changeably in the present disclosure to refer to plants with improved productivity under nitrogen limiting conditions.

As used herein “water use efficiency” refers to the amount of carbon dioxide assimilated by leaves per unit of water vapor transpired. It constitutes one of the most important traits controlling plant productivity in dry environments. “Drought tolerance” refers to the degree to which a plant is adapted to arid or drought conditions. The physiological responses of plants to a deficit of water include leaf wilting, a reduction in leaf area, leaf abscission, and the stimulation of root growth by directing nutrients to the underground parts of the plants. Typically, plants are more susceptible to drought during flowering and seed development (the reproductive stages), as plant's resources are deviated to support root growth. In addition, abscisic acid (ABA), a plant stress hormone, induces the closure of leaf stomata (microscopic pores involved in gas exchange), thereby reducing water loss through transpiration, and decreasing the rate of photosynthesis. These responses improve the water-use efficiency of the plant on the short term. The terms “increased water use efficiency”, “enhanced water use efficiency”, and “increased drought tolerance” are used inter-changeably in the present disclosure to refer to plants with improved productivity under water-limiting conditions.

As used herein “increased water use efficiency” refers to the ability of plants to grow, develop, or yield faster or better than normal when subjected to the same amount of available/applied water as under normal or standard conditions; ability of plants to grow, develop, or yield normally, or grow, develop, or yield faster or better when subjected to reduced amounts of available/applied water (water input) or under conditions of water stress or water deficit stress.

As used herein “increased drought tolerance” refers to the ability of plants to grow, develop, or yield normally, or grow, develop, or yield faster or better than normal when subjected to reduced amounts of available/applied water and/or under conditions of acute or chronic drought; ability of plants to grow, develop, or yield normally when subjected to reduced amounts of available/applied water (water input) or under conditions of water deficit stress or under conditions of acute or chronic drought.

As used herein, “drought stress” refers to a period of dryness (acute or chronic/prolonged) that results in water deficit and subjects plants to stress and/or damage to plant tissues and/or negatively affects grain/crop yield; a period of dryness (acute or chronic/prolonged) that results in water deficit and/or higher temperatures and subjects plants to stress and/or damage to plant tissues and/or negatively affects grain/crop yield.

As used herein, “water deficit” refers to the conditions or environments that provide less than optimal amounts of water needed for adequate/successful growth and development of plants.

As used herein, “water stress” refers to the conditions or environments that provide improper (either less/insufficient or more/excessive) amounts of water than that needed for adequate/successful growth and development of plants/crops thereby subjecting the plants to stress and/or damage to plant tissues and/or negatively affecting grain/crop yield.

As used herein “water deficit stress” refers to the conditions or environments that provide less/insufficient amounts of water than that needed for adequate/successful growth and development of plants/crops thereby subjecting the plants to stress and/or damage to plant tissues and/or negatively affecting grain yield.

As used herein, the terms “nucleic acid,” “nucleic acid molecule,” “nucleotide sequence” and “polynucleotide” refer to RNA or DNA that is linear or branched, single or double stranded, or a hybrid thereof. The term also encompasses RNA/DNA hybrids. When dsRNA is produced synthetically, less common bases, such as inosine, 5-methylcytosine, 6-methyladenine, hypoxanthine and others can also be used for antisense, dsRNA, and ribozyme pairing. For example, polynucleotides that contain C-5 propyne analogues of uridine and cytidine have been shown to bind RNA with high affinity and to be potent antisense inhibitors of gene expression. Other modifications, such as modification to the phosphodiester backbone, or the 2′-hydroxy in the ribose sugar group of the RNA can also be made.

As used herein, the term “nucleotide sequence” refers to a heteropolymer of nucleotides or the sequence of these nucleotides from the 5′ to 3′ end of a nucleic acid molecule and includes DNA or RNA molecules, including cDNA, a DNA fragment or portion, genomic DNA, synthetic (e.g., chemically synthesized) DNA, plasmid DNA, mRNA, and anti-sense RNA, any of which can be single stranded or double stranded. The terms “nucleotide sequence” “nucleic acid,” “nucleic acid molecule,” “nucleic acid construct,” “oligonucleotide” and “polynucleotide” are also used interchangeably herein to refer to a heteropolymer of nucleotides. Nucleic acid molecules and/or nucleotide sequences provided herein are presented herein in the 5′ to 3′ direction, from left to right and are represented using the standard code for representing the nucleotide characters as set forth in the U.S. sequence rules, 37 CFR §§ 1.821-1.825 and the World Intellectual Property Organization (WIPO) Standard ST.25. A “5′ region” as used herein can mean the region of a polynucleotide that is nearest the 5′ end of the polynucleotide. Thus, for example, an element in the 5′ region of a polynucleotide can be located anywhere from the first nucleotide located at the 5′ end of the polynucleotide to the nucleotide located halfway through the polynucleotide. A “3′ region” as used herein can mean the region of a polynucleotide that is nearest the 3′ end of the polynucleotide. Thus, for example, an element in the 3′ region of a polynucleotide can be located anywhere from the first nucleotide located at the 3′ end of the polynucleotide to the nucleotide located halfway through the polynucleotide.

As used herein with respect to nucleic acids, the term “fragment” or “portion” refers to a nucleic acid that is reduced in length relative (e.g., reduced by 1, 2, 3, 4, 5, 6, 7, 8, 9, 10, 11, 12, 13, 14, 15, 16, 17, 18, 19, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 400, 450, 500, 550, 600, 650, 700, 750, 800, 850, 900, 950 or 1000 or more nucleotides or any range or value therein) to a reference nucleic acid and that comprises, consists essentially of and/or consists of a nucleotide sequence of contiguous nucleotides identical or almost identical (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical) to a corresponding portion of the reference nucleic acid. As an example, a nucleic acid encoding a CKX polypeptide may be reduced by 1, 2, 3, 4, 5, 6, 7, 8, 9, 10, 11, 12, 13, 14, 15, 16, 17, 18, 19, 20, 25 30, 35, 40, 45, 50, 55, 60, 65, 70, 75, 80, 85, 90, 100, 105, 110, 115, 120, 125, 130, 135, 140, 145, 150, 155, 160, 165, 170, 175, or 180, or more nucleotides or any range or value therein, which reduction can result in improved yield traits in a plant. Such a nucleic acid fragment may be, where appropriate, included in a larger polynucleotide of which it is a constituent. As a further example, a repeat sequence of guide nucleic acid of this invention may comprise a portion of a wild type CRISPR-Cas repeat sequence (e.g., a wild Type CRISR-Cas repeat; e.g., a repeat from the CRISPR Cas system of, for example, a Cas9, Cas12a (Cpf1), Cas12b, Cas12c (C2c3), Cas12d (CasY), Cas12e (CasX), Cas12g, Cas12h, Cas12i, C2c4, C2c5, C2c8, C2c9, C2c10, Cas14a, Cas14b, and/or a Cas14c, and the like).

In some embodiments, a nucleic acid fragment or portion may comprise, consist essentially of or consist of about 1, 2, 3, 4, 5, 6, 7, 8, 9, 10, 15, 20, 25, 30, 35, 40, 45, 50, 60, 70, 80, 90, 100, 150, 200, 250, 300, 350, 400, 450, 500, 550, 600, 660, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2300, 2400, 2500, 2600, 2700, 2800, 2900, 3000, 3500, 4000, 4500, 5000, 5500, 6000, 6500, 7000, 7500, 8000, 8100, 8185 or more or more consecutive nucleotides of a CKX nucleic acid, optionally about 2800 consecutive base pairs to about 8190 consecutive base pairs from the 3′ end or about 650 consecutive base pairs to about 1620 consecutive base pairs from 3′ end; optionally up to the full length of the CKX nucleic acid, which reduction can result in improved yield traits in a plant.

In some embodiments, a nucleic acid fragment or portion may be the result of a truncation of a CKX1 nucleic acid in which 5516 consecutive nucleotides may be deleted from genomic sequence, e.g., the entire 3′ end from nucleotide 1884 to 7399, and/or 1605 consecutive nucleotides from the coding sequence (cds), e.g., the entire 3′ end from nucleotide 28-1632. Thus, in some embodiments, a deletion results in a truncation of a CKX1 genomic sequence starting at about nucleotide 1880, 1881, 1882, 1883, 1884, 1885, 1886, 1887, 1888, 1889, 1890, 1895, 1900, 1905, 1910, 1915, 1920, 1925, 1930, 1935, 1940, 1945, 1950, 1960, 1970, 1980, 1990, 2000, 2010, 2020, 2030, 2040, 2041, 2045, 2050, or 2060 up to full length of the genomic sequence (e.g., up to nucleotide 7399) with reference to nucleotide position numbering of SEQ ID NO:72. In some embodiments, a deletion results in a truncation of a CKX1 coding sequence starting at about nucleotide 25, 26, 27, 28, 29, 30, 31, 32, 33, 34, 35, 36, 37, 38, 39, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, or 205 up to full length of the coding sequence (e.g., up to nucleotide 1632) with reference to nucleotide position numbering of SEQ ID NO:73.

In some embodiments, a nucleic acid fragment or portion may be the result of a truncation of a CKX2 nucleic acid in which 5115 consecutive nucleotides may be deleted from genomic sequence, e.g., the entire 3′ end from nucleotide 803 to 5917, and/or 1610 consecutive nucleotides from the coding sequence (cds), e.g., the entire 3′ end from nucleotide 38 to 1647. Thus, in some embodiments, a deletion results in a truncation of a CKX2 genomic sequence starting at about nucleotide 800, 801, 802, 803, 804, 805, 806, 807, 808, 809, 810, 815, 820, 830, 840, 850, 860, 870, 880, 890, 900, 905, 910, 915, 920, 925, 930, 935, 940, 945, 950, or 955 up to full length of the genomic sequence (e.g., up to nucleotide 5917) with reference to nucleotide position numbering of SEQ ID NO:75. In some embodiments, a deletion results in a truncation of a CKX2 coding sequence starting at about nucleotide 35, 36, 37, 38, 39, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, or 190, up to full length of the coding sequence (e.g., up to nucleotide 1647) with reference to nucleotide position numbering of SEQ ID NO:76.

In some embodiments, a nucleic acid fragment or portion may be the result of a truncation of a CKX3 nucleic acid in which 5076 consecutive nucleotides may be deleted from genomic sequence, e.g., the entire 3′ end from nucleotide 692 to 5768, and/or 1574 consecutive nucleotides from the coding sequence (cds), e.g., the entire 3′ end from nucleotide 35 to 1608. Thus, in some embodiments, a deletion results in a truncation of a CKX3 genomic sequence starting at about nucleotide 690, 691, 692, 693, 694, 695, 670, 680, 690, 700, 710, 715, 720, 730, 740, 750, 760, 770, 780, 790, 800, 805, 810, 815, 820, 825, or 862 up to full length of the genomic sequence (e.g., up to nucleotide 5768) with reference to nucleotide position numbering of SEQ ID NO:78. In some embodiments, a deletion results in a truncation of a CKX3 coding sequence starting at about nucleotide 35, 36, 37, 38, 39, 40, 45, 50, 55, 60, 65, 70, 75, 80, 85, 90, 95, 100, 105, 110, 115, 120, 125, 130, 135, 140, 145, 150, 155, 160, 165, 166, 167, 168, or 169, up to full length of the coding sequence (e.g., up to nucleotide 1608) with reference to nucleotide position numbering of SEQ ID NO:79.

In some embodiments, a nucleic acid fragment or portion may be the result of a truncation of a CKX4 nucleic acid in which 8186 consecutive nucleotides may be deleted from genomic sequence, e.g., the entire 3′ end from nucleotide 1540-9725, and/or 1574 consecutive nucleotides from the coding sequence (cds), e.g., the entire 3′ end from nucleotide 2 to 1575. Thus, in some embodiments, a deletion results in a truncation of a CKX4 genomic sequence starting at about nucleotide 1540, 1541, 1542, 1543, 1544, 1545, 1546, 1547, 1548, 1549, 1550, 1555, 1560, 1570, 1580, 1590, 1600, 1610, 1620, 1630, 1640, 1645, 1650, 1660, 1670, 1680, 1685, 1686, 1687, 1688, 1689, 1690, 1700, 1800, 2000, 2500, 3000 up to full length of the genomic sequence (e.g., up to nucleotide 9725) with reference to nucleotide position numbering of SEQ ID NO:81. In some embodiments, a deletion results in a truncation of a CKX4 coding sequence starting at about nucleotide 2, 3, 4, 5, 6, 7, 8, 9, 10, 15, 20, 25, 30, 35, 40, 45, 50, 60, 70, 80, 90, 100, 105, 110, 115, 120, 125, 130, 135, 140, 145, 150, 151, 151, 152, 153, 154, or 155, up to full length of the coding sequence (e.g., up to nucleotide 1575) with reference to nucleotide position numbering of SEQ ID NO:82.

In some embodiments, a nucleic acid fragment or portion may be the result of a truncation of a CKX5 (error) nucleic acid in which 2972 consecutive nucleotides may be deleted from genomic sequence, e.g., the entire 3′ end from nucleotide 690-3661), and/or 678 consecutive nucleotides from the coding sequence (cds), e.g., the entire 3′ end from nucleotide 43 to 720. Thus, in some embodiments, a deletion results in a truncation of a CKX5 genomic sequence starting at about nucleotide 690, 691, 692, 693, 695, 696, 697, 698, 699, 700, 705, 710, 715, 720, 725, 730, 735, 740, 745, 750, 755, 760, 765, 770, 775, 780, 785, or 790 up to full length of the genomic sequence (e.g., up to nucleotide 3661) with reference to nucleotide position numbering of SEQ ID NO:84. In some embodiments, a deletion results in a truncation of a CKX5 coding sequence starting at about nucleotide 40, 41, 42, 43, 44, 45, 46, 47, 48, 49, 50, 55, 60, 65, 70, 75, 80, 85, 90, 95, 100, 105, 110, 25, 30, 35, 40, 45, 50, 60, 70, 80, 90, 100, 105, 110, 115, 120, 125, 130, 135, 140, 141, 142, 143, 144, or 145 up to full length of the coding sequence (e.g., up to nucleotide 720) with reference to nucleotide position numbering of SEQ ID NO:91.

In some embodiments, a nucleic acid fragment or portion may be the result of a truncation of a CKX5 nucleic acid in which 3338 consecutive nucleotides may be deleted from genomic sequence, e.g., the entire 3′ end from nucleotide 658-3995, and/or 1563 consecutive nucleotides from the coding sequence (cds), e.g., the entire 3′ end from nucleotide 43 to 1605. Thus, in some embodiments, a deletion results in a truncation of a CKX5 genomic sequence starting at about nucleotide 655, 656, 657, 658, 659, 660, 665, 670, 675, 680, 685, 690, 691, 692, 693, 695, 696, 697, 698, 699, 700, 705, 710, 715, 720, 725, 730, 735, 740, 745, 750, 755, 756, 757, or 758 up to full length of the genomic sequence (e.g., up to nucleotide 3995) with reference to nucleotide position numbering of SEQ ID NO:84. In some embodiments, a deletion results in a truncation of a CKX5 coding sequence starting at about nucleotide 40, 41, 42, 43, 44, 45, 46, 47, 48, 49, 50, 55, 60, 65, 70, 75, 80, 85, 90, 95, 100, 105, 110, 25, 30, 35, 40, 45, 50, 60, 70, 80, 90, 100, 105, 110, 115, 120, 125, 130, 135, 140, 141, 142, or 143 up to full length of the coding sequence (e.g., up to nucleotide 1605) with reference to nucleotide position numbering of SEQ ID NO:91.

In some embodiments, a nucleic acid fragment or portion may be the result of a truncation of a CKX6 nucleic acid in which 6716 consecutive nucleotides may be deleted from genomic sequence, e.g., the entire 3′ end from nucleotide 31 to 1494, and/or 678 consecutive nucleotides from the coding sequence (cds), e.g., the entire 3′ end from nucleotide 31 to 1494. Thus, in some embodiments, a deletion results in a truncation of a CKX6 genomic sequence starting at about nucleotide 1560, 1561, 1562, 1563, 1565, 1566, 1567, 1568, 1569, 1570, 1575, 1580, 1585, 1590, 1595, 1600, 1610, 1620, 1630, 1640, 1645, 1650, 1655, 1660, 1665, 1670, 1675, 1680, 1685, 1690, 1695, 1700, 1705, 1706, 1707, 1708, or 1709 up to full length of the genomic sequence (e.g., up to nucleotide 8277) with reference to nucleotide position numbering of SEQ ID NO:87. In some embodiments, a deletion results in a truncation of a CKX6 coding sequence starting at about nucleotide 30, 31, 32, 33, 34, 35, 36, 37, 38, 39, 40, 45, 50, 55, 60, 65, 70, 75, 80, 85, 90, 95, 100, 105, 110, 25, 30, 35, 40, 45, 50, 60, 70, 80, 90, 100, 105, 110, 115, 120, 125, 130, 135, 140, 145, 150, 155, 160, 165, 170, 175, 176, 177, 178 up to full length of the coding sequence (e.g., up to nucleotide 1494) with reference to nucleotide position numbering of SEQ ID NO:88.

As used herein with respect to polypeptides, the term “fragment” or “portion” may refer to a polypeptide that is reduced in length relative to a reference polypeptide and that comprises, consists essentially of and/or consists of an amino acid sequence of contiguous amino acids identical or almost identical (e.g., 90%, 91%, 92%, 93%, 94%, 95%, 96%, 97%, 98%, 99% identical) to a corresponding portion of the reference polypeptide. Such a polypeptide fragment may be, where appropriate, included in a larger polypeptide of which it is a constituent. In some embodiments, the polypeptide fragment comprises, consists essentially of or consists of at least about 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100, 125, 150, 175, 200, 225, 250, 300, 350, 400 or more consecutive amino acids of a reference polypeptide.

In some embodiments, a “portion” may be related to the number of amino acids that are deleted from a polypeptide. Thus, for example, a deleted “portion” of a CKX polypeptide may comprise at least one amino acid residue (e.g., at least 1, or at least 2, 3, 4, 5, 6, 7, 8, 9, 10, 20, 30, 40, 50, 100, 150, 200, 250, 300, 350, 400, 450, 500 or more consecutive amino acid residues) deleted from the amino acid sequence of any one of SEQ ID NOs:74, 77, 80, 83, 89, or 92 (or from a sequence having at least 80% sequence identity (e.g., at least 80, 81, 82, 83, 84, 85, 86, 87, 88, 89, 90, 91, 92, 93, 94, 95, 96, 97, 98, 99 or 100% identity) to an amino acid sequence of any one of SEQ ID NOs:74, 77, 80, 83, 89, or 92).

In some embodiments, a deletion of amino acid residues from a CKX polypeptide (a truncated CKX polypeptide or loss of the CKX polypeptide) as described herein may result in a dominant negative mutation, a semi-dominant mutation, a weak loss-of-function mutation, a hypomorphic mutation, or a null mutation, which when comprised in a plant can result in the plant exhibiting improved yield traits as compared to a plant not comprising the deletion.

A “region” of a polynucleotide or a polypeptide refers to a portion of consecutive nucleotides or consecutive amino acid residues of that polynucleotide or a polypeptide, respectively. For example, a region of a CKX polynucleotide sequence may include, but is not limited to, consecutive nucleotides 1884-2060 of SEQ ID NO:72, consecutive nucleotides 28-204 of SEQ ID NO:73, consecutive nucleotides 803-955 of SEQ ID NO:75, consecutive nucleotides 38-190 of SEQ ID NO:76, consecutive nucleotides 692-826 of SEQ ID NO:78, consecutive nucleotides 35-169 of SEQ ID NO:79, consecutive nucleotides 1540-1689 of SEQ ID NO:81 consecutive nucleotides 2-151 of SEQ ID NO:82, consecutive nucleotides 690-790 of SEQ ID NO:84, consecutive nucleotides 1562-1709 of SEQ ID NO:87 consecutive nucleotides 31-178 of SEQ ID NO:88, and/or consecutive nucleotides 43-143 of SEQ ID NO:91 (see e.g., SEQ ID NOs:78, 79, 80, or 81), and a region of a CKX polypeptide may include, but is not limited to, amino acid residues 588-601 of SEQ ID NO:74 or SEQ ID NO:77 (see, e.g., SEQ ID NO:93, 94, 95, 96, 97, 98, or 99).

In some embodiments, a “sequence-specific nucleic acid binding domain” or “sequence-specific DNA binding domain” may bind to a CKX gene (e.g., SEQ ID NO:72, SEQ ID NO:73, SEQ ID NO:75, SEQ ID NO:76, SEQ ID NO:78, SEQ ID NO:79, SEQ ID NO:81, SEQ ID NO:82, SEQ ID NO:84, SEQ ID NO:87, SEQ ID NO:88, or SEQ ID NO:91) and/or to one or more fragments, portions, or regions of a CKX nucleic acid (e.g., SEQ ID NOs:93-97).

As used herein with respect to nucleic acids, the term “functional fragment” refers to nucleic acid that encodes a functional fragment of a polypeptide.

The term “gene,” as used herein, refers to a nucleic acid molecule capable of being used to produce mRNA, antisense RNA, miRNA, anti-microRNA antisense oligodeoxyribonucleotide (AMO) and the like. Genes may or may not be capable of being used to produce a functional protein or gene product. Genes can include both coding and non-coding regions (e.g., introns, regulatory elements, promoters, enhancers, termination sequences and/or 5′ and 3′ untranslated regions). A gene may be “isolated” by which is meant a nucleic acid that is substantially or essentially free from components normally found in association with the nucleic acid in its natural state. Such components include other cellular material, culture medium from recombinant production, and/or various chemicals used in chemically synthesizing the nucleic acid.

The term “mutation” refers to point mutations (e.g., missense, or nonsense, or insertions or deletions of single base pairs that result in in-frame shifts), insertions, deletions, and/or truncations. When the mutation is a substitution of a residue within an amino acid sequence with another residue, or a deletion or insertion of one or more residues within a sequence, the mutations are typically described by identifying the original residue followed by the position of the residue within the sequence and by the identity of the newly substituted residue In some embodiments, a deletion or an insertion is an in-frame deletion or an in-frame insertion. In some embodiments, a deletion may result in a frameshift mutation that generates a premature stop codon, thereby truncating the protein. In some embodiments, a deletion may result in a frameshift mutation that generates a premature stop codon, thereby truncating the protein. In some embodiments, a frameshift mutation is an out-of-frame mutation. In some embodiments, a frameshift mutation may be an in-frame mutation. A truncation can include a truncation at the C-terminal end of a polypeptide or at the N-terminal end of a polypeptide. A truncation of a polypeptide can be the result of a deletion in the corresponding 5′ end or 3′ end of the gene encoding the polypeptide. In some embodiments, the truncation of a CKX polypeptide is a C-terminal truncation that results from a deletion that occurs/initiates in the 5′ end of a CKX gene (e.g., a mutation that results in a premature stop codon), wherein the truncation results in an N-terminal fragment of the CKX polypeptide, optionally no polypeptide. In some embodiments, a mutation in an endogenous CKX gene may result in an inactive CKX polypeptide. In some embodiments, a mutation in the promoter (e.g., promoter bashing) of an endogenous CKX gene may result in modified (increased/decreased) expression of the CKX gene, and therefore an increased amount of the CKX polypeptide. In some embodiments, a mutation in an endogenous CKX gene may result in reduced expression of the CKX gene, and therefore a reduced amount of the CKX polypeptide that is a null or inactive polypeptide. An endogenous CKX gene mutated as described herein may have the same level of expression as a WT CKX gene, but the mutated gene produces a null or inactive CKX polypeptide.

The terms “complementary” or “complementarity,” as used herein, refer to the natural binding of polynucleotides under permissive salt and temperature conditions by base-pairing. For example, the sequence “A-G-T” (5′ to 3′) binds to the complementary sequence “T-C-A” (3′ to 5′). Complementarity between two single-stranded molecules may be “partial,” in which only some of the nucleotides bind, or it may be complete when total complementarity exists between the single stranded molecules. The degree of complementarity between nucleic acid strands has significant effects on the efficiency and strength of hybridization between nucleic acid strands.

“Complement,” as used herein, can mean 100% complementarity with the comparator nucleotide sequence or it can mean less than 100% complementarity (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and the like, complementarity; e.g., substantial complementarity) to the comparator nucleotide sequence.

Different nucleic acids or proteins having homology are referred to herein as “homologues.” The term homologue includes homologous sequences from the same and from other species and orthologous sequences from the same and other species. “Homology” refers to the level of similarity between two or more nucleic acid and/or amino acid sequences in terms of percent of positional identity (i.e., sequence similarity or identity). Homology also refers to the concept of similar functional properties among different nucleic acids or proteins. Thus, the compositions and methods of the invention further comprise homologues to the nucleotide sequences and polypeptide sequences of this invention. “Orthologous,” as used herein, refers to homologous nucleotide sequences and/or amino acid sequences in different species that arose from a common ancestral gene during speciation. A homologue of a nucleotide sequence of this invention has a substantial sequence identity (e.g., at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100%) to said nucleotide sequence of the invention.

As used herein “sequence identity” refers to the extent to which two optimally aligned polynucleotide or polypeptide sequences are invariant throughout a window of alignment of components, e.g., nucleotides or amino acids. “Identity” can be readily calculated by known methods including, but not limited to, those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991).

As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned. In some embodiments, “percent sequence identity” can refer to the percentage of identical amino acids in an amino acid sequence as compared to a reference polypeptide.

As used herein, the phrase “substantially identical,” or “substantial identity” in the context of two nucleic acid molecules, nucleotide sequences or polypeptide sequences, refers to two or more sequences or subsequences that have at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100% nucleotide or amino acid residue identity, when compared and aligned for maximum correspondence, as measured using one of the following sequence comparison algorithms or by visual inspection. In some embodiments of the invention, the substantial identity exists over a region of consecutive nucleotides of a nucleotide sequence of the invention that is about 10 nucleotides to about 20 nucleotides, about 10 nucleotides to about 25 nucleotides, about 10 nucleotides to about 30 nucleotides, about 15 nucleotides to about 25 nucleotides, about 30 nucleotides to about 40 nucleotides, about 50 nucleotides to about 60 nucleotides, about 70 nucleotides to about 80 nucleotides, about 90 nucleotides to about 100 nucleotides, about 100 nucleotides to about 200 nucleotides, about 100 nucleotides to about 300 nucleotides, about 100 nucleotides to about 400 nucleotides, about 100 nucleotides to about 500 nucleotides, about 100 nucleotides to about 600 nucleotides, about 100 nucleotides to about 800 nucleotides, about 100 nucleotides to about 900 nucleotides, or more nucleotides in length, and any range therein, up to the full length of the sequence. In some embodiments, nucleotide sequences can be substantially identical over at least about 20 consecutive nucleotides (e.g., about 20, 21, 22, 23, 24, 25, 26, 27, 28, 29, 30, 31, 32, 33, 34, 35, 36, 37, 38, 39, 40, 50, 60, 70, 80, 90, 100, 200, 300, 400, 500, 600, 700, 800, 900, 1000, 1100, 1200, 1300, 1400, 1500, 1600, 1700, 1800, 1900, 2000, 2100, 2200, 2300, 2500, 3000, 3500, 4000 or more nucleotides). In some embodiments, two or more CKX genes may be substantially identical to one another over at least about 50, 100, 200, 300, 400, 500, 600, 700, 800, 900, 1000, 1100, 1200, 1300, 1400, or 1500 to about 2000, 2050, 2100, 2150, 2200, 2250, 2300, 2350, 2400, 2450, 2500, 2510, 2520, 2530, 2540, 2550, 2600, 2650, 2700, 2750, 2800, 2850, 2900, 2950, 3000, 3050, 3100, 3150, 3200, 3250, 3300, 3350, 3400, 3410, or 3420 or more consecutive nucleotides of SEQ ID NO:72, SEQ ID NO:73, SEQ ID NO:75, SEQ ID NO:76, SEQ ID NO:78, SEQ ID NO:79, SEQ ID NO:81, SEQ ID NO:82, SEQ ID NO:84, SEQ ID NO:87, SEQ ID NO:88, or SEQ ID NO:91, over at least about 100, 110, 120, 130, 140, or 150 to about 200, 210, 220, 230, 240, 250, 260, 270, 280, 290, 300, 400, 500, 600, 700, 750, 800, 810, 820, 830, 840, 850, 900, 1000, 1250, 1500, 1750, 2000, 2500, 3000, or 3500 or more consecutive nucleotides SEQ ID NO:72, SEQ ID NO:73, SEQ ID NO:75, SEQ ID NO:76, SEQ ID NO:78, SEQ ID NO:79, SEQ ID NO:81, SEQ ID NO:82, SEQ ID NO:84, SEQ ID NO:87, SEQ ID NO:88, or SEQ ID NO:91.

In some embodiments of the invention, the substantial identity exists over a region of consecutive amino acid residues of a polypeptide of the invention that is about 3 amino acid residues to about 10, 11, 12, 13, 14, 15, 16, 17, 18, 19, or 20 amino acid residues, about 5 amino acid residues to about 25, 30, 35, 40, 45, 50 or 60 amino acid residues, about 15 amino acid residues to about 30 amino acid residues, about 20 amino acid residues to about 40 amino acid residues, about 25 amino acid residues to about 40 amino acid residues, about 25 amino acid residues to about 50 amino acid residues, about 30 amino acid residues to about 50 amino acid residues, about 40 amino acid residues to about 50 amino acid residues, about 40 amino acid residues to about 70 amino acid residues, about 50 amino acid residues to about 70 amino acid residues, about 60 amino acid residues to about 80 amino acid residues, about 70 amino acid residues to about 80 amino acid residues, about 90 amino acid residues to about 100 amino acid residues, or more amino acid residues in length, and any range therein, up to the full length of the sequence. In some embodiments, polypeptide sequences can be substantially identical to one another over at least about 8, 9, 10, 11, 12, 13, 14, or more consecutive amino acid residues (e.g., about 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 130, 140, 150, 175, 200, 225, 250, 275, 300, 325, 350, 400, 450, 500, or more amino acids in length or more consecutive amino acid residues). In some embodiments, two or more CKX polypeptides may be substantially identical to one another over at least about 10 to about 500 consecutive amino acid residues of any one of the amino acid sequences of SEQ ID NOs:74, 77, 80, 83, 89, or 92; e.g., over at least about 10, 11, 12, 13, 14, 15, 16, 17, 18, 19, 20, 25, 30, 35, 40, 45, 50, 60, 70, 71, 72, 73, 74, 75, 76, 77, 78, 79, 80, 85, 90, 95, 100, 105, 110, 120, 125, 130, 135, 140, 145, 150, 155, 160, 165, 170, 175, 200, 225, 250, 275, 300, 325, 350, 400, 450, or 500 consecutive amino acid residues of any one of the amino acid sequences of SEQ ID NOs:74, 77, 80, 83, 89, or 92. In some embodiments, a substantially identical nucleotide or protein sequence may perform substantially the same function as the nucleotide (or encoded protein sequence) to which it is substantially identical.

For sequence comparison, typically one sequence acts as a reference sequence to which test sequences are compared. When using a sequence comparison algorithm, test and reference sequences are entered into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCG® Wisconsin Package® (Accelrys Inc., San Diego, Calif.). An “identity fraction” for aligned segments of a test sequence and a reference sequence is the number of identical components which are shared by the two aligned sequences divided by the total number of components in the reference sequence segment, e.g., the entire reference sequence or a smaller defined part of the reference sequence. Percent sequence identity is represented as the identity fraction multiplied by 100. The comparison of one or more polynucleotide sequences may be to a full-length polynucleotide sequence or a portion thereof, or to a longer polynucleotide sequence. For purposes of this invention “percent identity” may also be determined using BLASTX version 2.0 for translated nucleotide sequences and BLASTN version 2.0 for polynucleotide sequences.

Two nucleotide sequences may also be considered substantially complementary when the two sequences hybridize to each other under stringent conditions. In some embodiments, two nucleotide sequences considered to be substantially complementary hybridize to each other under highly stringent conditions.

“Stringent hybridization conditions” and “stringent hybridization wash conditions” in the context of nucleic acid hybridization experiments such as Southern and Northern hybridizations are sequence dependent and are different under different environmental parameters. An extensive guide to the hybridization of nucleic acids is found in Tijssen Laboratory Techniques in Biochemistry and Molecular Biology-Hybridization with Nucleic Acid Probes part I chapter 2 “Overview of principles of hybridization and the strategy of nucleic acid probe assays” Elsevier, New York (1993). Generally, highly stringent hybridization and wash conditions are selected to be about 5° C. lower than the thermal melting point (Tₘ) for the specific sequence at a defined ionic strength and pH.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleotide sequences which have more than 100 complementary residues on a filter in a Southern or northern blot is 50% formamide with 1 mg of heparin at 42° C., with the hybridization being carried out overnight. An example of highly stringent wash conditions is 0.1 5M NaCl at 72° C. for about 15 minutes. An example of stringent wash conditions is a 0.2×SSC wash at 65° C. for 15 minutes (see, Sambrook, infra, for a description of SSC buffer). Often, a high stringency wash is preceded by a low stringency wash to remove background probe signal. An example of a medium stringency wash for a duplex of, e.g., more than 100 nucleotides, is 1×SSC at 45° C. for 15 minutes. An example of a low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.0 M Na ion, typically about 0.01 to 1.0 M Na ion concentration (or other salts) at pH 7.0 to 8.3, and the temperature is typically at least about 30° C. Stringent conditions can also be achieved with the addition of destabilizing agents such as formamide. In general, a signal to noise ratio of 2× (or higher) than that observed for an unrelated probe in the particular hybridization assay indicates detection of a specific hybridization. Nucleotide sequences that do not hybridize to each other under stringent conditions are still substantially identical if the proteins that they encode are substantially identical. This can occur, for example, when a copy of a nucleotide sequence is created using the maximum codon degeneracy permitted by the genetic code.

A polynucleotide and/or recombinant nucleic acid construct of this invention (e.g., expression cassettes and/or vectors) may be codon optimized for expression. In some embodiments, the polynucleotides, nucleic acid constructs, expression cassettes, and/or vectors of the editing systems of the invention (e.g., comprising/encoding a sequence-specific nucleic acid binding domain (e.g., a sequence-specific DNA binding domain from a polynucleotide-guided endonuclease, a zinc finger nuclease, a transcription activator-like effector nuclease (TALEN), an Argonaute protein, and/or a CRISPR-Cas endonuclease (e.g., CRISPR-Cas effector protein) (e.g., a Type I CRISPR-Cas effector protein, a Type II CRISPR-Cas effector protein, a Type III CRISPR-Cas effector protein, a Type IV CRISPR-Cas effector protein, a Type V CRISPR-Cas effector protein or a Type VI CRISPR-Cas effector protein)), a nuclease (e.g., an endonuclease (e.g., Fok1), a polynucleotide-guided endonuclease, a CRISPR-Cas endonuclease (e.g., CRISPR-Cas effector protein), a zinc finger nuclease, and/or a transcription activator-like effector nuclease (TALEN)), deaminase proteins/domains (e.g., adenine deaminase, cytosine deaminase), a polynucleotide encoding a reverse transcriptase protein or domain, a polynucleotide encoding a 5′-3′ exonuclease polypeptide, and/or affinity polypeptides, peptide tags, etc.) may be codon optimized for expression in a plant. In some embodiments, the codon optimized nucleic acids, polynucleotides, expression cassettes, and/or vectors of the invention have about 70% to about 99.9% (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%. 99.9% or 100%) identity or more to the reference nucleic acids, polynucleotides, expression cassettes, and/or vectors that have not been codon optimized.

A polynucleotide or nucleic acid construct of the invention may be operatively associated with a variety of promoters and/or other regulatory elements for expression in a plant and/or a cell of a plant. Thus, in some embodiments, a polynucleotide or nucleic acid construct of this invention may further comprise one or more promoters, introns, enhancers, and/or terminators operably linked to one or more nucleotide sequences. In some embodiments, a promoter may be operably associated with an intron (e.g., Ubi1 promoter and intron). In some embodiments, a promoter associated with an intron maybe referred to as a “promoter region” (e.g., Ubi1 promoter and intron) (see, e.g., SEQ ID NO:21 and SEQ ID NO:22).

By “operably linked” or “operably associated” as used herein in reference to polynucleotides, it is meant that the indicated elements are functionally related to each other and are also generally physically related. Thus, the term “operably linked” or “operably associated” as used herein, refers to nucleotide sequences on a single nucleic acid molecule that are functionally associated. Thus, a first nucleotide sequence that is operably linked to a second nucleotide sequence means a situation when the first nucleotide sequence is placed in a functional relationship with the second nucleotide sequence. For instance, a promoter is operably associated with a nucleotide sequence if the promoter effects the transcription or expression of said nucleotide sequence. Those skilled in the art will appreciate that the control sequences (e.g., promoter) need not be contiguous with the nucleotide sequence to which it is operably associated, as long as the control sequences function to direct the expression thereof. Thus, for example, intervening untranslated, yet transcribed, nucleic acid sequences can be present between a promoter and the nucleotide sequence, and the promoter can still be considered “operably linked” to the nucleotide sequence.

As used herein, the term “linked,” in reference to polypeptides, refers to the attachment of one polypeptide to another. A polypeptide may be linked to another polypeptide (at the N-terminus or the C-terminus) directly (e.g., via a peptide bond) or through a linker.

The term “linker” is art-recognized and refers to a chemical group, or a molecule linking two molecules or moieties, e.g., two domains of a fusion protein, such as, for example, a nucleic acid/DNA binding polypeptide or domain and peptide tag and/or a reverse transcriptase and an affinity polypeptide that binds to the peptide tag; or a DNA endonuclease polypeptide or domain and peptide tag and/or a reverse transcriptase and an affinity polypeptide that binds to the peptide tag. A linker may be comprised of a single linking molecule or may comprise more than one linking molecule. In some embodiments, the linker can be an organic molecule, group, polymer, or chemical moiety such as a bivalent organic moiety. In some embodiments, the linker may be an amino acid or it may be a peptide. In some embodiments, the linker is a peptide.

In some embodiments, a peptide linker useful with this invention may be about 2 to about 100 or more amino acids in length, for example, about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more amino acids in length (e.g., about 2 to about 40, about 2 to about 50, about 2 to about 60, about 4 to about 40, about 4 to about 50, about 4 to about 60, about 5 to about 40, about 5 to about 50, about 5 to about 60, about 9 to about 40, about 9 to about 50, about 9 to about 60, about 10 to about 40, about 10 to about 50, about 10 to about 60, or about 2, 3, 4, 5, 6, 7, 8, 9, 10, 11, 12, 13, 14, 15, 16, 17, 18, 19, 20, 21, 22, 23, 24, 25 amino acids to about 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more amino acids in length (e.g., about 105, 110, 115, 120, 130, 140 150 or more amino acids in length). In some embodiments, a peptide linker may be a GS linker.

As used herein, the term “linked,” or “fused” in reference to polynucleotides, refers to the attachment of one polynucleotide to another. In some embodiments, two or more polynucleotide molecules may be linked by a linker that can be an organic molecule, group, polymer, or chemical moiety such as a bivalent organic moiety. A polynucleotide may be linked or fused to another polynucleotide (at the 5′ end or the 3′ end) via a covalent or non-covenant linkage or binding, including e.g., Watson-Crick base-pairing, or through one or more linking nucleotides. In some embodiments, a polynucleotide motif of a certain structure may be inserted within another polynucleotide sequence (e.g., extension of the hairpin structure in the guide RNA). In some embodiments, the linking nucleotides may be naturally occurring nucleotides. In some embodiments, the linking nucleotides may be non-naturally occurring nucleotides.

A “promoter” is a nucleotide sequence that controls or regulates the transcription of a nucleotide sequence (e.g., a coding sequence) that is operably associated with the promoter. The coding sequence controlled or regulated by a promoter may encode a polypeptide and/or a functional RNA. Typically, a “promoter” refers to a nucleotide sequence that contains a binding site for RNA polymerase II and directs the initiation of transcription. In general, promoters are found 5′, or upstream, relative to the start of the coding region of the corresponding coding sequence. A promoter may comprise other elements that act as regulators of gene expression;

e.g., a promoter region. These include a TATA box consensus sequence, and often a CAAT box consensus sequence (Breathnach and Chambon, (1981) Annu. Rev. Biochem. 50:349). In plants, the CAAT box may be substituted by the AGGA box (Messing et al., (1983) in Genetic Engineering of Plants, T. Kosuge, C. Meredith and A. Hollaender (eds.), Plenum Press, pp. 211-227).

Promoters useful with this invention can include, for example, constitutive, inducible, temporally regulated, developmentally regulated, chemically regulated, tissue-preferred and/or tissue-specific promoters for use in the preparation of recombinant nucleic acid molecules, e.g., “synthetic nucleic acid constructs” or “protein-RNA complex.” These various types of promoters are known in the art.

The choice of promoter may vary depending on the temporal and spatial requirements for expression, and also may vary based on the host cell to be transformed. Promoters for many different organisms are well known in the art. Based on the extensive knowledge present in the art, the appropriate promoter can be selected for the particular host organism of interest. Thus, for example, much is known about promoters upstream of highly constitutively expressed genes in model organisms and such knowledge can be readily accessed and implemented in other systems as appropriate.

In some embodiments, a promoter functional in a plant may be used with the constructs of this invention. Non-limiting examples of a promoter useful for driving expression in a plant include the promoter of the RubisCo small subunit gene 1 (PrbcS1), the promoter of the actin gene (Pactin), the promoter of the nitrate reductase gene (Pnr) and the promoter of duplicated carbonic anhydrase gene 1 (Pdcal) (See, Walker et al. Plant Cell Rep. 23:727-735 (2005); Li et al. Gene 403:132-142 (2007); Li et al. Mol Biol. Rep. 37:1143-1154 (2010)). PrbcS1 and Pactin are constitutive promoters and Pnr and Pdcal are inducible promoters. Pnr is induced by nitrate and repressed by ammonium (Li et al. Gene 403:132-142 (2007)) and Pdcal is induced by salt (Li et al. Mol Biol. Rep. 37:1143-1154 (2010)). In some embodiments, a promoter useful with this invention is RNA polymerase II (Pol II) promoter. In some embodiments, a U6 promoter or a 7SL promoter from Zea mays may be useful with constructs of this invention. In some embodiments, the U6c promoter and/or 7SL promoter from Zea mays may be useful for driving expression of a guide nucleic acid. In some embodiments, a U6c promoter, U6i promoter and/or 7SL promoter from Glycine max may be useful with constructs of this invention. In some embodiments, the U6c promoter, U6i promoter and/or 7SL promoter from Glycine max may be useful for driving expression of a guide nucleic acid.

Examples of constitutive promoters useful for plants include, but are not limited to, cestrum virus promoter (cmp) (U.S. Pat. No. 7,166,770), the rice actin 1 promoter (Wang et al. (1992) Mol. Cell. Biol. 12:3399-3406; as well as U.S. Pat. No. 5,641,876), CaMV 35S promoter (Odell et al. (1985) Nature 313:810-812), CaMV 19S promoter (Lawton et al. (1987) Plant Mol. Biol. 9:315-324), nos promoter (Ebert et al. (1987) Proc. Natl. Acad. Sci USA 84:5745-5749), Adh promoter (Walker et al. (1987) Proc. Natl. Acad. Sci. USA 84:6624-6629), sucrose synthase promoter (Yang & Russell (1990) Proc. Natl. Acad. Sci. USA 87:4144-4148), and the ubiquitin promoter. The constitutive promoter derived from ubiquitin accumulates in many cell types. Ubiquitin promoters have been cloned from several plant species for use in transgenic plants, for example, sunflower (Binet et al., 1991. Plant Science 79: 87-94), maize (Christensen et al., 1989. Plant Molec. Biol. 12: 619-632), and Arabidopsis (Norris et al. 1993. Plant Molec. Biol. 21:895-906). The maize ubiquitin promoter (UbiP) has been developed in transgenic monocot systems and its sequence and vectors constructed for monocot transformation are disclosed in the patent publication EP 0 342 926. The ubiquitin promoter is suitable for the expression of the nucleotide sequences of the invention in transgenic plants, especially monocotyledons. Further, the promoter expression cassettes described by McElroy et al. (Mol. Gen. Genet. 231: 150-160 (1991)) can be easily modified for the expression of the nucleotide sequences of the invention and are particularly suitable for use in monocotyledonous hosts.

In some embodiments, tissue specific/tissue preferred promoters can be used for expression of a heterologous polynucleotide in a plant cell. Tissue specific or preferred expression patterns include, but are not limited to, green tissue specific or preferred, root specific or preferred, stem specific or preferred, flower specific or preferred or pollen specific or preferred.

Promoters suitable for expression in green tissue include many that regulate genes involved in photosynthesis and many of these have been cloned from both monocotyledons and dicotyledons. In one embodiment, a promoter useful with the invention is the maize PEPC promoter from the phosphoenol carboxylase gene (Hudspeth & Grula, Plant Molec. Biol. 12:579-589 (1989)). Non-limiting examples of tissue-specific promoters include those associated with genes encoding the seed storage proteins (such as β-conglycinin, cruciferin, napin and phaseolin), zein or oil body proteins (such as oleosin), or proteins involved in fatty acid biosynthesis (including acyl carrier protein, stearoyl-ACP desaturase and fatty acid desaturases (fad 2-1)), and other nucleic acids expressed during embryo development (such as Bce4, see, e.g., Kridl et al. (1991) Seed Sci. Res. 1:209-219; as well as EP Patent No. 255378). Tissue-specific or tissue-preferential promoters useful for the expression of the nucleotide sequences of the invention in plants, particularly maize, include but are not limited to those that direct expression in root, pith, leaf or pollen. Such promoters are disclosed, for example, in WO 93/07278, herein incorporated by reference in its entirety. Other non-limiting examples of tissue specific or tissue preferred promoters useful with the invention the cotton rubisco promoter disclosed in U.S. Pat. No. 6,040,504; the rice sucrose synthase promoter disclosed in U.S. Pat. No. 5,604,121; the root specific promoter described by de Framond (FEBS 290:103-106 (1991); EP 0 452 269 to Ciba-Geigy); the stem specific promoter described in U.S. Pat. No. 5,625,136 (to Ciba-Geigy) and which drives expression of the maize trpA gene; the cestrum yellow leaf curling virus promoter disclosed in WO 01/73087; and pollen specific or preferred promoters including, but not limited to, ProOsLPS10 and ProOsLPS11 from rice (Nguyen et al. Plant Biotechnol. Reports 9(5):297-306 (2015)), ZmSTK2_USP from maize (Wang et al. Genome 60(6):485-495 (2017)), LAT52 and LAT59 from tomato (Twell et al. Development 109(3):705-713 (1990)), Zm13 (U.S. Pat. No. 10,421,972), PLA₂-δ promoter from Arabidopsis (U.S. Pat. No. 7,141,424), and/or the ZmC5 promoter from maize (International PCT Publication No. WO1999/042587.

Additional examples of plant tissue-specific/tissue preferred promoters include, but are not limited to, the root hair-specific cis-elements (RHEs) (Kim et al. The Plant Cell 18:2958-2970 (2006)), the root-specific promoters RCc3 (Jeong et al. Plant Physiol. 153:185-197 (2010)) and RB7 (U.S. Pat. No. 5,459,252), the lectin promoter (Lindstrom et al. (1990) Der. Genet. 11:160-167; and Vodkin (1983) Prog. Clin. Biol. Res. 138:87-98), corn alcohol dehydrogenase 1 promoter (Dennis et al. (1984) Nucleic Acids Res. 12:3983-4000), S-adenosyl-L-methionine synthetase (SAMS) (Vander Mijnsbrugge et al. (1996) Plant and Cell Physiology, 37(8):1108-1115), corn light harvesting complex promoter (Bansal et al. (1992) Proc. Natl. Acad. Sci. USA 89:3654-3658), corn heat shock protein promoter (O'Dell et al. (1985) EMBO J. 5:451-458; and Rochester et al. (1986) EMBO J. 5:451-458), pea small subunit RuBP carboxylase promoter (Cashmore, “Nuclear genes encoding the small subunit of ribulose-1,5-bisphosphate carboxylase” pp. 29-39 In: Genetic Engineering of Plants (Hollaender ed., Plenum Press 1983; and Poulsen et al. (1986) Mol. Gen. Genet. 205:193-200), Ti plasmid mannopine synthase promoter (Langridge et al. (1989) Proc. Natl. Acad. Sci. USA 86:3219-3223), Ti plasmid nopaline synthase promoter (Langridge et al. (1989), supra), petunia chalcone isomerase promoter (van Tunen et al. (1988) EMBO J. 7:1257-1263), bean glycine rich protein 1 promoter (Keller et al. (1989) Genes Dev. 3:1639-1646), truncated CaMV 35S promoter (O'Dell et al. (1985) Nature 313:810-812), potato patatin promoter (Wenzler et al. (1989) Plant Mol. Biol. 13:347-354), root cell promoter (Yamamoto et al. (1990) Nucleic Acids Res. 18:7449), maize zein promoter (Kriz et al. (1987) Mol. Gen. Genet. 207:90-98; Langridge et al. (1983) Cell 34:1015-1022; Reina et al. (1990) Nucleic Acids Res. 18:6425; Reina et al. (1990) Nucleic Acids Res. 18:7449; and Wandelt et al. (1989) Nucleic Acids Res. 17:2354), globulin-1 promoter (Belanger et al. (1991) Genetics 129:863-872), α-tubulin cab promoter (Sullivan et al. (1989)Mol. Gen. Genet. 215:431-440), PEPCase promoter (Hudspeth & Grula (1989) Plant Mol. Biol. 12:579-589), R gene complex-associated promoters (Chandler et al. (1989) Plant Cell 1:1175-1183), and chalcone synthase promoters (Franken et al. (1991) EMBO J. 10:2605-2612).

Useful for seed-specific expression is the pea vicilin promoter (Czako et al. (1992) Mol. Gen. Genet. 235:33-40; as well as the seed-specific promoters disclosed in U.S. Pat. No. 5,625,136. Useful promoters for expression in mature leaves are those that are switched at the onset of senescence, such as the SAG promoter from Arabidopsis (Gan et al. (1995) Science 270:1986-1988).

In addition, promoters functional in chloroplasts can be used. Non-limiting examples of such promoters include the bacteriophage T3 gene 9 5′ UTR and other promoters disclosed in U.S. Pat. No. 7,579,516. Other promoters useful with the invention include but are not limited to the S-E9 small subunit RuBP carboxylase promoter and the Kunitz trypsin inhibitor gene promoter (Kti3).

Additional regulatory elements useful with this invention include, but are not limited to, introns, enhancers, termination sequences and/or 5′ and 3′ untranslated regions.

An intron useful with this invention can be an intron identified in and isolated from a plant and then inserted into an expression cassette to be used in transformation of a plant. As would be understood by those of skill in the art, introns can comprise the sequences required for self-excision and are incorporated into nucleic acid constructs/expression cassettes in frame. An intron can be used either as a spacer to separate multiple protein-coding sequences in one nucleic acid construct, or an intron can be used inside one protein-coding sequence to, for example, stabilize the mRNA. If they are used within a protein-coding sequence, they are inserted “in-frame” with the excision sites included. Introns may also be associated with promoters to improve or modify expression. As an example, a promoter/intron combination useful with this invention includes but is not limited to that of the maize Ubi1 promoter and intron (see, e.g., SEQ ID NO:21 and SEQ ID NO:22).

Non-limiting examples of introns useful with the present invention include introns from the ADHI gene (e.g., Adh1-S introns 1, 2 and 6), the ubiquitin gene (Ubi1), the RuBisCO small subunit (rbcS) gene, the RuBisCO large subunit (rbcL) gene, the actin gene (e.g., actin-1 intron), the pyruvate dehydrogenase kinase gene (pdk), the nitrate reductase gene (nr), the duplicated carbonic anhydrase gene 1 (Tdca1), the psbA gene, the atpA gene, or any combination thereof.

In some embodiments, a polynucleotide and/or a nucleic acid construct of the invention can be an “expression cassette” or can be comprised within an expression cassette. As used herein, “expression cassette” means a recombinant nucleic acid molecule comprising, for example, a one or more polynucleotides of the invention (e.g., a polynucleotide encoding a sequence-specific nucleic acid binding domain, a polynucleotide encoding a deaminase protein or domain, a polynucleotide encoding a reverse transcriptase protein or domain, a polynucleotide encoding a 5′-3′ exonuclease polypeptide or domain, a guide nucleic acid and/or reverse transcriptase (RT) template), wherein polynucleotide(s) is/are operably associated with one or more control sequences (e.g., a promoter, terminator and the like). Thus, in some embodiments, one or more expression cassettes may be provided, which are designed to express, for example, a nucleic acid construct of the invention (e.g., a polynucleotide encoding a sequence-specific nucleic acid binding domain, a polynucleotide encoding a nuclease polypeptide/domain, a polynucleotide encoding a deaminase protein/domain, a polynucleotide encoding a reverse transcriptase protein/domain, a polynucleotide encoding a 5′-3′ exonuclease polypeptide/domain, a polynucleotide encoding a peptide tag, and/or a polynucleotide encoding an affinity polypeptide, and the like, or comprising a guide nucleic acid, an extended guide nucleic acid, and/or RT template, and the like). When an expression cassette of the present invention comprises more than one polynucleotide, the polynucleotides may be operably linked to a single promoter that drives expression of all of the polynucleotides or the polynucleotides may be operably linked to one or more separate promoters (e.g., three polynucleotides may be driven by one, two or three promoters in any combination). When two or more separate promoters are used, the promoters may be the same promoter or they may be different promoters. Thus, a polynucleotide encoding a sequence specific nucleic acid binding domain, a polynucleotide encoding a nuclease protein/domain, a polynucleotide encoding a CRISPR-Cas effector protein/domain, a polynucleotide encoding an deaminase protein/domain, a polynucleotide encoding a reverse transcriptase polypeptide/domain (e.g., RNA-dependent DNA polymerase), and/or a polynucleotide encoding a 5′-3′ exonuclease polypeptide/domain, a guide nucleic acid, an extended guide nucleic acid and/or RT template when comprised in a single expression cassette may each be operably linked to a single promoter, or separate promoters in any combination.

An expression cassette comprising a nucleic acid construct of the invention may be chimeric, meaning that at least one of its components is heterologous with respect to at least one of its other components (e.g., a promoter from the host organism operably linked to a polynucleotide of interest to be expressed in the host organism, wherein the polynucleotide of interest is from a different organism than the host or is not normally found in association with that promoter). An expression cassette may also be one that is naturally occurring but has been obtained in a recombinant form useful for heterologous expression.

An expression cassette can optionally include a transcriptional and/or translational termination region (i.e., termination region) and/or an enhancer region that is functional in the selected host cell. A variety of transcriptional terminators and enhancers are known in the art and are available for use in expression cassettes. Transcriptional terminators are responsible for the termination of transcription and correct mRNA polyadenylation. A termination region and/or the enhancer region may be native to the transcriptional initiation region, may be native to, for example, a gene encoding a sequence-specific nucleic acid binding protein, a gene encoding a nuclease, a gene encoding a reverse transcriptase, a gene encoding a deaminase, and the like, or may be native to a host cell, or may be native to another source (e.g., foreign or heterologous to, for example, to a promoter, to a gene encoding a sequence-specific nucleic acid binding protein, a gene encoding a nuclease, a gene encoding a reverse transcriptase, a gene encoding a deaminase, and the like, or to the host cell, or any combination thereof).

An expression cassette of the invention also can include a polynucleotide encoding a selectable marker, which can be used to select a transformed host cell. As used herein, “selectable marker” means a polynucleotide sequence that when expressed imparts a distinct phenotype to the host cell expressing the marker and thus allows such transformed cells to be distinguished from those that do not have the marker. Such a polynucleotide sequence may encode either a selectable or screenable marker, depending on whether the marker confers a trait that can be selected for by chemical means, such as by using a selective agent (e.g., an antibiotic and the like), or on whether the marker is simply a trait that one can identify through observation or testing, such as by screening (e.g., fluorescence). Many examples of suitable selectable markers are known in the art and can be used in the expression cassettes described herein.

In addition to expression cassettes, the nucleic acid molecules/constructs and polynucleotide sequences described herein can be used in connection with vectors. The term “vector” refers to a composition for transferring, delivering or introducing a nucleic acid (or nucleic acids) into a cell. A vector comprises a nucleic acid construct (e.g., expression cassette(s)) comprising the nucleotide sequence(s) to be transferred, delivered or introduced. Vectors for use in transformation of host organisms are well known in the art. Non-limiting examples of general classes of vectors include viral vectors, plasmid vectors, phage vectors, phagemid vectors, cosmid vectors, fosmid vectors, bacteriophages, artificial chromosomes, minicircles, or Agrobacterium binary vectors in double or single stranded linear or circular form which may or may not be self-transmissible or mobilizable. In some embodiments, a viral vector can include, but is not limited, to a retroviral, lentiviral, adenoviral, adeno-associated, or herpes simplex viral vector. A vector as defined herein can transform a prokaryotic or eukaryotic host either by integration into the cellular genome or exist extrachromosomally (e.g., autonomous replicating plasmid with an origin of replication). Additionally included are shuttle vectors by which is meant a DNA vehicle capable, naturally or by design, of replication in two different host organisms, which may be selected from actinomycetes and related species, bacteria and eukaryotic (e.g., higher plant, mammalian, yeast or fungal cells). In some embodiments, the nucleic acid in the vector is under the control of, and operably linked to, an appropriate promoter or other regulatory elements for transcription in a host cell. The vector may be a bi-functional expression vector which functions in multiple hosts. In the case of genomic DNA, this may contain its own promoter and/or other regulatory elements and in the case of cDNA this may be under the control of an appropriate promoter and/or other regulatory elements for expression in the host cell. Accordingly, a nucleic acid or polynucleotide of this invention and/or expression cassettes comprising the same may be comprised in vectors as described herein and as known in the art.

As used herein, “contact,” “contacting,” “contacted,” and grammatical variations thereof, refer to placing the components of a desired reaction together under conditions suitable for carrying out the desired reaction (e.g., transformation, transcriptional control, genome editing, nicking, and/or cleavage). As an example, a target nucleic acid may be contacted with a sequence-specific nucleic acid binding protein (e.g., polynucleotide-guided endonuclease, a CRISPR-Cas endonuclease (e.g., CRISPR-Cas effector protein), a zinc finger nuclease, a transcription activator-like effector nuclease (TALEN) and/or an Argonaute protein)) and a deaminase or a nucleic acid construct encoding the same, under conditions whereby the sequence-specific nucleic acid binding protein, the reverse transcriptase and the deaminase are expressed and the sequence-specific nucleic acid binding protein binds to the target nucleic acid, and the reverse transcriptase and/or deaminase may be fused to either the sequence-specific nucleic acid binding protein or recruited to the sequence-specific nucleic acid binding protein (via, for example, a peptide tag fused to the sequence-specific nucleic acid binding protein and an affinity tag fused to the reverse transcriptase and/or deaminase) and thus, the deaminase and/or reverse transcriptase is positioned in the vicinity of the target nucleic acid, thereby modifying the target nucleic acid. Other methods for recruiting reverse transcriptase and/or deaminase may be used that take advantage of other protein-protein interactions. In addition, RNA-protein interactions and chemical interactions may be used for protein-protein and protein-nucleic acid recruitment.

As used herein, “modifying” or “modification” in reference to a target nucleic acid includes editing (e.g., mutating), covalent modification, exchanging/substituting nucleic acids/nucleotide bases, deleting, cleaving, nicking, and/or altering transcriptional control of a target nucleic acid. In some embodiments, a modification may include one or more single base changes (SNPs) of any type.

The term “regulating” as used in the context of a polypeptide “regulating” a phenotype, for example, a balance between inactive and active cytokinins in a plant, means the ability of the polypeptide to affect the expression of a gene or genes such that a phenotype such as the cytokinin balance is modified.

“Introducing,” “introduce,” “introduced” (and grammatical variations thereof) in the context of a polynucleotide of interest means presenting a nucleotide sequence of interest (e.g., polynucleotide, RT template, a nucleic acid construct, and/or a guide nucleic acid) to a plant, plant part thereof, or cell thereof, in such a manner that the nucleotide sequence gains access to the interior of a cell.

The terms “transformation” or transfection” may be used interchangeably and as used herein refer to the introduction of a heterologous nucleic acid into a cell. Transformation of a cell may be stable or transient. Thus, in some embodiments, a host cell or host organism (e.g., a plant) may be stably transformed with a polynucleotide/nucleic acid molecule of the invention. In some embodiments, a host cell or host organism may be transiently transformed with a polynucleotide/nucleic acid molecule of the invention.

“Transient transformation” in the context of a polynucleotide means that a polynucleotide is introduced into the cell and does not integrate into the genome of the cell.

By “stably introducing” or “stably introduced” in the context of a polynucleotide introduced into a cell is intended that the introduced polynucleotide is stably incorporated into the genome of the cell, and thus the cell is stably transformed with the polynucleotide.

“Stable transformation” or “stably transformed” as used herein means that a nucleic acid molecule is introduced into a cell and integrates into the genome of the cell. As such, the integrated nucleic acid molecule is capable of being inherited by the progeny thereof, more particularly, by the progeny of multiple successive generations. “Genome” as used herein includes the nuclear and the plastid genome, and therefore includes integration of the nucleic acid into, for example, the chloroplast or mitochondrial genome. Stable transformation as used herein can also refer to a transgene that is maintained extrachromosomally, for example, as a minichromosome or a plasmid.

Transient transformation may be detected by, for example, an enzyme-linked immunosorbent assay (ELISA) or Western blot, which can detect the presence of a peptide or polypeptide encoded by one or more transgene introduced into an organism. Stable transformation of a cell can be detected by, for example, a Southern blot hybridization assay of genomic DNA of the cell with nucleic acid sequences which specifically hybridize with a nucleotide sequence of a transgene introduced into an organism (e.g., a plant). Stable transformation of a cell can be detected by, for example, a Northern blot hybridization assay of RNA of the cell with nucleic acid sequences which specifically hybridize with a nucleotide sequence of a transgene introduced into a host organism. Stable transformation of a cell can also be detected by, e.g., a polymerase chain reaction (PCR) or other amplification reactions as are well known in the art, employing specific primer sequences that hybridize with target sequence(s) of a transgene, resulting in amplification of the transgene sequence, which can be detected according to standard methods Transformation can also be detected by direct sequencing and/or hybridization protocols well known in the art.

Accordingly, in some embodiments, nucleotide sequences, polynucleotides, nucleic acid constructs, and/or expression cassettes of the invention may be expressed transiently and/or they can be stably incorporated into the genome of the host organism. Thus, in some embodiments, a nucleic acid construct of the invention (e.g., one or more expression cassettes comprising polynucleotides for editing as described herein) may be transiently introduced into a cell with a guide nucleic acid and as such, no DNA is maintained in the cell.

A nucleic acid construct of the invention may be introduced into a plant cell by any method known to those of skill in the art. Non-limiting examples of transformation methods include transformation via bacterial-mediated nucleic acid delivery (e.g., via Agrobacteria), viral-mediated nucleic acid delivery, silicon carbide or nucleic acid whisker-mediated nucleic acid delivery, liposome mediated nucleic acid delivery, microinjection, microparticle bombardment, calcium-phosphate-mediated transformation, cyclodextrin-mediated transformation, electroporation, nanoparticle-mediated transformation, sonication, infiltration, PEG-mediated nucleic acid uptake, as well as any other electrical, chemical, physical (mechanical) and/or biological mechanism that results in the introduction of nucleic acid into the plant cell, including any combination thereof. Procedures for transforming both eukaryotic and prokaryotic organisms are well known and routine in the art and are described throughout the literature (See, for example, Jiang et al. 2013. Nat. Biotechnol. 31:233-239; Ran et al. Nature Protocols 8:2281-2308 (2013)). General guides to various plant transformation methods known in the art include Miki et al. (“Procedures for Introducing Foreign DNA into Plants” in Methods in Plant Molecular Biology and Biotechnology, Glick, B. R. and Thompson, J. E., Eds. (CRC Press, Inc., Boca Raton, 1993), pages 67-88) and Rakowoczy-Trojanowska (Cell. Mol. Biol. Lett. 7:849-858 (2002)).

In some embodiments of the invention, transformation of a cell may comprise nuclear transformation. In other embodiments, transformation of a cell may comprise plastid transformation (e.g., chloroplast transformation). In still further embodiments, nucleic acids of the invention may be introduced into a cell via conventional breeding techniques. In some embodiments, one or more of the polynucleotides, expression cassettes and/or vectors may be introduced into a plant cell via Agrobacterium transformation.

A polynucleotide therefore can be introduced into a plant, plant part, plant cell in any number of ways that are well known in the art. The methods of the invention do not depend on a particular method for introducing one or more nucleotide sequences into a plant, only that they gain access to the interior the cell. Where more than polynucleotide is to be introduced, they can be assembled as part of a single nucleic acid construct, or as separate nucleic acid constructs, and can be located on the same or different nucleic acid constructs. Accordingly, the polynucleotide can be introduced into the cell of interest in a single transformation event, or in separate transformation events, or, alternatively, a polynucleotide can be incorporated into a plant as part of a breeding protocol.

Cytokinins are phytohormones that are involved in numerous physiological processes in plants and their levels may be a target for modification of yield in plants. For example, cytokinin oxidase (CKX) activity in plants is increased during times of abiotic stress, which leads to an increase in inactive cytokinins and decreased plant productivity. Targeted manipulation of the cytokinin balance (e.g., relative balance of active and inactive cytokinins) in targeted tissue types and developmental stages through modification of endogenous genes encoding CKX polypeptides may provide increased productivity (e.g., improved yield traits). CKX is a flavoprotein in which the FAD cofactor is covalently linked to a histidine residue. Such increased productivity may be possible even under abiotic stress conditions, through mechanisms such as increased cell division, induction of stomatal opening, inhibited senescence of organs, and/or suppression of apical dominance.

Accordingly, in some embodiments, the present invention is directed to generating mutations in endogenous CKX genes, optionally wherein the mutation results in the production of an altered amount of CKX polypeptide, a truncated CKX polypeptide or no CKX polypeptide. In some embodiments, a mutation in an endogenous CKX gene or two or more endogenous CKX genes can result in modifying the balance between inactive cytokinins versus active cytokinins in favor of active cytokinins, thereby improving yield traits in the plant. Thus, in some embodiments, the mutations as described herein result in an increase in the supply of active cytokinin to a tissue of interest, e.g., in the reproductive organs of a plant. In some embodiments, supply of active cytokinin to a tissue may be increased or altered (e.g., increased or decreased) during particular stages of development or in a particular tissue type.

In some embodiments, the present invention provides a plant or plant part thereof comprising at least one non-natural mutation in at least one endogenous Cytokinin Oxidase/Dehydrogenase (CKX) gene encoding a CKX protein. In some embodiments, a mutation in an endogenous CKX gene results in an inactive CKX polypeptide. In some embodiments, a mutation in the promoter (e.g., promoter bashing) of an endogenous CKX gene may result in modified (increased/decreased) expression of the CKX gene, and therefore an increased amount of the CKX polypeptide. In some embodiments, a mutation in an endogenous CKX gene may result in reduced expression of the gene as compared to a WT CKX gene, and therefore a reduced amount of a null or inactive CKX polypeptide. In some embodiments, a mutated CKX gene as described herein may have the same level of expression as the WT CKX gene, but the mutated CKX gene produces a null or inactive CKX polypeptide. In some embodiments, the CKX gene is a CKX1 gene, a CKX2 gene, a CKX3 gene, a CKX4 gene, a CKX5 gene, and/or a CKX6 gene. In some embodiments, the at least one non-natural mutation is a mutation in two or more CKX genes (e.g., 2, 3, 4, 5, or 6 CKX genes), e.g., a mutation in two or more of a CKX1 gene, a CKX2 gene, a CKX3 gene, a CKX4 gene, a CKX5 gene, and/or a CKX6 gene, in any combination. In some embodiments, the at least one non-natural mutation is a mutation in at least three (e.g., 3, 4, 5, or 6) of the endogenous CKX genes of CKX1, CKX2, CKX3, CKX4, CKX5 and/or CKX6 gene, in any combination. In some embodiments, a plant or plant part thereof comprising at least one non-natural mutation in at least one endogenous CKX gene encoding a CKX protein comprises a mutation (a) in an endogenous CKX1 gene, an endogenous CKX2 gene, and an endogenous CKX3 gene; (b) in an endogenous CKX1 gene, an endogenous CKX3, an endogenous CKX5 gene, and an endogenous CKX6 gene; or (c) in an endogenous CKX1 gene, an endogenous CKX2 gene, an endogenous CKX3 gene, and an endogenous CKX4 gene.

In some embodiments, a plant comprising at least one non-natural mutation in at least one endogenous CKX gene encoding a CKX protein has improved yield traits compared to an isogenic plant (e.g., wild type unedited plant or a null segregant) that does not comprise the mutation.

In some embodiments, an endogenous CKX gene: (a) comprises a sequence having at least 80% sequence identity to any one of the nucleotide sequences of SEQ ID NOs:72, 73, 75, 76, 78, 79, 81, 82, 84, 87, 88, or 91; (b) comprises a region having at least 80% sequence identity to any one of the nucleotide sequences of SEQ ID NOs:93-98; and/or (c) encodes a polypeptide having at least 80% identity to any one of the amino acid sequences of SEQ ID NOs: 74, 77, 80, 83, 89, or 92. In some embodiments, an endogenous CKX gene is a CKX1 gene that (a) comprises a sequence having at least 80% sequence identity to the nucleotide sequence of SEQ ID NO:72 or SEQ ID NO:73; (b) comprises a region having at least 80% sequence identity to any one of the nucleotide sequences of SEQ ID NO:93; and/or (c) encodes a polypeptide comprising a sequence having at least 80% sequence identity to the amino acid sequence of SEQ ID NO:74. In some embodiments, an endogenous CKX gene is a CKX2 gene that (a) comprises a sequence having at least 80% sequence identity to the nucleotide sequence of SEQ ID NO:75 or SEQ ID NO:76; (b) comprises a region having at least 80% sequence identity to the nucleotide sequence of SEQ ID NO:94; and/or (c) encodes a polypeptide comprising a sequence having at least 80% sequence identity to the amino acid sequence of SEQ ID NO:77. In some embodiments, an endogenous CKX gene is a CKX3 gene that (a) comprises a sequence having at least 80% sequence identity to the nucleotide sequence of SEQ ID NO:78 or SEQ ID NO:79; (b) comprises a region having at least 80% sequence identity to the nucleotide sequence of SEQ ID NO:95; and/or (c) encodes a polypeptide comprising a sequence having at least 80% sequence identity to the amino acid sequence of SEQ ID NO:80. In some embodiments, an endogenous CKX gene is a CKX4 gene that (a) comprises a sequence having at least 80% sequence identity to the nucleotide sequence of SEQ ID NO:81 or SEQ ID NO:82; (b) comprises a region having at least 80% sequence identity to the nucleotide sequence of SEQ ID NO:96; and/or (c) encodes a polypeptide comprising a sequence having at least 80% sequence identity to the amino acid sequence of SEQ ID NO:83. In some embodiments, an endogenous CKX gene is a CKX5 gene that (a) comprises a sequence having at least 80% sequence identity to the nucleotide sequence of SEQ ID NO:84 or SEQ ID NO:91; (b) comprises a region having at least 80% sequence identity to the nucleotide sequence of SEQ ID NO:97; and/or (c) encodes a polypeptide comprising a sequence having at least 80% sequence identity to the amino acid sequence of SEQ ID NO:92. In some embodiments, an endogenous CKX gene is a CKX6 gene that (a) comprises a sequence having at least 80% sequence identity to the nucleotide sequence of SEQ ID NO:87 or SEQ ID NO:88; (b) comprises a region having at least 80% sequence identity to the nucleotide sequence of SEQ ID NO:98; and/or (c) encodes a polypeptide comprising a sequence having at least 80% sequence identity to the amino acid sequence of SEQ ID NO:89.

Thus, in some embodiments, a plant or plant part of the invention comprises at least one non-natural mutation in an endogenous CKX gene, wherein the endogenous CKX gene (a) comprises a sequence having at least 80% sequence identity to any one of the nucleotide sequences of SEQ ID NOs:72, 73, 75, 76, 78, 79, 81, 82, 84, 87, 88, or 91; (b) comprises a region having at least 80% sequence identity to any one of the nucleotide sequences of SEQ ID NOs:93-98; and/or (c) encodes a polypeptide having at least 80% identity to any one of the amino acid sequences of SEQ ID NOs: 74, 77, 80, 83, 89, or 92.

A non-natural mutation in an endogenous Cytokinin Oxidase/Dehydrogenase (CKX) gene in a plant may be any type of mutation including, but not limited to, a point mutation, a base substitution, a base deletion and/or a base insertion, optionally wherein the at least one non-natural mutation results in a premature stop codon. In some embodiments, a plant comprising an endogenous CKX gene that has at least one non-natural mutation in a CKX gene as described herein exhibits improved yield traits as compared to a plant that does not comprise the at least one non-natural mutation in a CKX gene.

A mutation useful with this invention can include, but is not limited to, a substitution, a deletion and/or an insertion of one or more bases of the CKX gene or a deletion or substitution of one or more amino acid residues of the CKX polypeptide. In some embodiments, the at least one non-natural mutation results in a premature stop codon. In some embodiments, at least one non-natural mutation may comprise a base substitution to an A, a T, a G, or a C, which results in premature stop codon, thereby generating a truncated CKX polypeptide. In some embodiments, a premature stop codon results in a truncation of the CKX polypeptide such that no CKX polypeptide is produced. In some embodiments, a mutation in an endogenous CKX gene may result in altered expression (e.g., increased or decreased expression) of the gene as compared to a wild type CKX gene, and therefore an altered amount of the CKX polypeptide compared to the corresponding wild type CKX gene (e.g., the CKX gene not modified as described herein). In some embodiments, a mutation in the promoter (e.g., promoter bashing) of an endogenous CKX gene may result in modified (increased/decreased) expression of the CKX gene, and therefore an increased amount of the CKX polypeptide. In some embodiments, a mutation in an endogenous CKX gene may result in reduced expression of the gene as compared to a wild type CKX gene, and therefore a reduced amount of the CKX polypeptide that is a null or inactive polypeptide. In some embodiments, a mutated CKX gene as described herein may have the same expression level as the wild type CKX gene, but the mutated CKX gene produces a null or inactive CKX polypeptide.

In some embodiments, the at least one non-natural mutation in an endogenous CKX gene may be a deletion (e.g., a deletion of one or more consecutive base pairs, e.g., at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 100, 200, 300, 400, 500, 600, 700, 800, 900, 1000, 1500, 2000, 2500, 3000, 3500, 4000, 4500, 5000, 6000, 7000, or 8000 or more consecutive base pairs of any one of SEQ ID NOs: 72, 73, 75, 76, 78, 79, 81, 82, 84, 87, 88, or 91). In some embodiments, an endogenous CKX gene comprises a deletion of at least one or two or more consecutive base pairs, or at least three consecutive base pairs. In some embodiments, an endogenous CKX gene comprises a deletion of at least one base pair that results in a truncated CKX polypeptide (e.g., C-terminal truncation) or no CKX polypeptide.

In some embodiments, at least one non-natural mutation may produce a dominant negative mutation, a semi-dominant mutation, a weak loss-of-function mutation, a hypomorphic mutation, or a null mutation. In some embodiments, the at least one non-natural mutation is a null mutation. In some embodiments, the at least one non-natural mutation is a dominant negative mutation. In some embodiments, the at least one non-natural mutation is a semi-dominant mutation. In some embodiments, a non-natural mutation in an endogenous gene encoding a CKX polypeptide useful with this invention may be a dominant recessive mutation. In some embodiments, a plant comprising the null mutation and/or the dominant negative mutation exhibits improved yield traits (e.g., increased pod production, increased seed production, increased seed size, increased seed weight, increased nodule number, increase nodule activity, and/or increased nitrogen fixation) as compared to a control plant (e.g., a plant not comprising the dominant negative mutation and/or null mutation).

In some embodiments, a plant cell comprising an editing system is provided, the editing system comprising: (a) a CRISPR-associated effector protein; and (b) a guide nucleic acid (gRNA, gDNA, crRNA, crDNA, sgRNA, sgDNA) comprising a spacer sequence with complementarity to an endogenous target gene encoding a CKX protein in the plant cell. In some embodiments, the editing system generates a mutation in the endogenous target gene encoding a CKX protein. The endogenous target gene encoding a CKX protein may be any CKX protein involved in (e.g., capable of influencing or regulating) the relative balance between active and inactive cytokinins (optionally increasing the active cytokinins relative to the inactive cytokinins) and may be modified to increase yield components such as pod production/number, seed production/number, seed size, and/or seed weight. In some embodiments, an endogenous target gene encoding a CKX protein is an endogenous CKX1 gene, an endogenous CKX2 gene, an endogenous CKX3 gene, an endogenous CKX4 gene, an endogenous CKX5 gene, or an endogenous CKX6 gene, or any combination thereof. In some embodiments, an endogenous gene encoding a CKX protein and to which the spacer sequence of the guide nucleic acid is complementary comprises a sequence having at least 80% sequence identity to any one of the nucleotide sequences of SEQ ID NOs: 72, 73, 75, 76, 78, 79, 81, 82, 84, 87, 88, or 91, and/or comprises a region having at least 80% sequence identity to any one of the nucleotide sequences of SEQ ID NOs:93-98. In some embodiments, CKX protein encoded by the endogenous gene comprises at least 80% sequence identity to any one of the amino acid sequences SEQ ID NOs:74, 77, 80, 83, 89, or 92. In some embodiments, a spacer sequence useful with this invention can include, but is not limited to, a nucleotide sequence of any one of SEQ ID NOs:99-113.

In some embodiments, a plant cell is provided comprising at least one non-natural mutation within an endogenous Cytokinin Oxidase/Dehydrogenase (CKX) gene that results in a null allele or knockout of the CKX gene, wherein the at least one non-natural mutation is a base substitution, base insertion or a base deletion that is introduced using an editing system that comprises a nucleic acid binding domain that binds to a target site in the CKX gene. In some embodiments, the nuclease is a zinc finger nuclease, a transcription activator-like effector nuclease (TALEN), an endonuclease (e.g., Fok1) or a CRISPR-Cas effector protein. In some embodiments, the nucleic acid binding domain of the editing system is from a polynucleotide-guided endonuclease, a CRISPR-Cas endonuclease (e.g., CRISPR-Cas effector protein), a zinc finger nuclease, a transcription activator-like effector nuclease (TALEN) and/or an Argonaute protein. In some embodiments, the endogenous CKX gene is a CKX1 gene, a CKX2 gene, a CKX3 gene, a CKX4 gene, a CKX5 gene, and/or a CKX6 gene, or any combination thereof, optionally wherein the at least one non-natural mutation is a mutation in at least two (e.g., 2, 3, 4, 5, or 6) different endogenous CKX genes, in any combination (e.g., any combination of at least two of CKX1, CKX2, CKX3, CKX4, CKX5 or CKX6). In some embodiments, the at least one non-natural mutation is a mutation in (a) in an endogenous CKX1 gene, an endogenous CKX2 gene, and an endogenous CKX3 gene; (b) in an endogenous CKX1 gene, an endogenous CKX3, an endogenous CKX5 gene, and an endogenous CKX6 gene; or (c) in an endogenous CKX1 gene, an endogenous CKX2 gene, an endogenous CKX3 gene, and an endogenous CKX4 gene.

In some embodiments, the endogenous CKX gene comprises a sequence having at least 80% sequence identity to any one of the nucleotide sequences of SEQ ID NOs:72, 73, 75, 76, 78, 79, 81, 82, 84, 87, 88, or 91, comprises a region having at least 80% sequence identity to any one of the nucleotide sequences of SEQ ID NOs:93-98 and/or encodes a polypeptide having at least 80% identity to any one of the amino acid sequences of SEQ ID NOs: 74, 77, 80, 83, 89, or 92. In some embodiments, a target site in a CKX gene is within a region of the CKX gene, the region comprising a sequence having at least 80% sequence identity to a sequence comprising: (a) about nucleotide 1884 to about nucleotide 2060 of the nucleotide sequence of SEQ ID NO:72 (CKX1) or about nucleotide 28 to about nucleotide 204 of the nucleotide sequence of SEQ ID NO:73 (CKX1) (e.g., SEQ ID NO:93); (b) about nucleotide 803 to about nucleotide 955 of the nucleotide sequence of SEQ ID NO:75 (CKX2) or about nucleotide 38 to about nucleotide 190 of the nucleotide sequence of SEQ ID NO:76 (CKX2) (e.g., SEQ ID NO:94); (c) about nucleotide 692 to about nucleotide 826 of the nucleotide sequence of SEQ ID NO:78 (CKX3) or about nucleotide 35 to about nucleotide 169 of the nucleotide sequence of SEQ ID NO:79 (CKX3) (e.g., SEQ ID NO:95); (d) about nucleotide 1540 to about nucleotide 1689 of the nucleotide sequence of SEQ ID NO:81 (CKX4) or about nucleotide 2 to about nucleotide 151 of the nucleotide sequence of SEQ ID NO:82 (CKX4) (e.g., SEQ ID NO:95); (e) about nucleotide 690 to about nucleotide 790 of the nucleotide sequence of SEQ ID NO:84 (CKX5), or about nucleotide 43 to about nucleotide 143 of the nucleotide sequence of SEQ ID NO:91 (CKX5) (e.g., SEQ ID NO:97); and/or (0 about nucleotide 1562 to about nucleotide 1709 of the nucleotide sequence of SEQ ID NO:87 (CKX6) or about nucleotide 31 to about nucleotide 178 (CKX6) of the nucleotide sequence of SEQ ID NO:88 (CKX6) (e.g., SEQ ID NO:98).

In some embodiments, the editing system further comprises a nuclease, and the nucleic acid binding domain binds to a target site in the CKX gene, wherein the CKX gene comprises a sequence having at least 80% sequence identity to any one of the nucleotide sequences of SEQ ID NOs:72, 73, 75, 76, 78, 79, 81, 82, 84, 87, 88, or 91, comprises a region having at least 80% sequence identity to any one of the nucleotide sequences of SEQ ID NOs:93-98, and/or encodes a polypeptide having at least 80% identity to any one of the amino acid sequences of SEQ ID NOs: 74, 77, 80, 83, 89, or 92, and the at least one non-natural mutation is made following cleavage by the nuclease. In some embodiments, the target site comprises a sequence having at least 80% sequence identity to any one of the nucleotide sequences of SEQ ID NOs:93-98.

In some embodiments, the at least one non-natural mutation is a point mutation. In some embodiments, a non-natural mutation can be a base substitution to an A, a T, a G, or a C, optionally wherein the base substitution results in an amino acid substitution. In some embodiments, the at least one non-natural mutation may be a base deletion or a base insertion of at least one or at least two (e.g., 2, 3, 4, 5, 6, 7, 8, 9, 10, 50, 100, 150, or 200 or more) consecutive bases. In some embodiments, the at least one non-natural mutation results in a deletion of all or a portion of the 5′ region of the CKX gene that results in a truncated CKX protein. In some embodiments, the at least one non-natural mutation results in a 3′ end truncation of the CKX gene, which produces a truncated CKX protein or no CKX protein. In some embodiments, the at least one non-natural mutation is a null allele or a dominant negative mutation.

Non-limiting examples of a plant or part thereof useful with this invention include corn, soy, canola, wheat, rice, cotton, sugarcane, sugar beet, barley, oats, alfalfa, sunflower, safflower, oil palm, sesame, coconut, tobacco, potato, sweet potato, cassava, coffee, apple, plum, apricot, peach, cherry, pear, fig, banana, citrus, cocoa, avocado, olive, almond, walnut, strawberry, watermelon, pepper, grape, tomato, cucumber, blackberry, raspberry, black raspberry, or a Brassica spp. In some embodiments, the plant or part thereof may be a soybean plant or part of a soybean plant.

In some embodiments, the plant part may be from a plant that includes, but is not limited to, corn, soy, canola, wheat, rice, cotton, sugarcane, sugar beet, barley, oats, alfalfa, sunflower, safflower, oil palm, sesame, coconut, tobacco, potato, sweet potato, cassava, coffee, apple, plum, apricot, peach, cherry, pear, fig, banana, citrus, cocoa, avocado, olive, almond, walnut, strawberry, watermelon, pepper, grape, tomato, cucumber, blackberry, raspberry, black raspberry or a Brassica spp. In some embodiments, a plant may be regenerated from a plant part of this invention including, for example, a cell. In some embodiments, a plant of this invention comprising at least one non-natural mutation in a CKX gene comprises improved yield traits.

In some embodiments, a soybean plant or part thereof is provided that comprises at least one non-natural mutation in an endogenous Cytokinin Oxidase/Dehydrogenase (CKX) gene having the gene identification number (gene ID) of Glyma15g18560, Glyma09g07360, Glyma17g06220, Glyma04g03130, Glyma09g35950 and/or Glyma09g07190.

Also provided herein is a method of providing a plurality of plants having improved yield traits (e.g., increased pod number, increased seed number, increased seed weight, increase nodule number, increase nodule activity, increase nitrogen fixation as a result of increase nodulation, or improved yield traits as a result of increased planting density), the method comprising planting two or more plants of the invention in a growing area, thereby providing a plurality of plants having improved yield traits as compared to a plurality of control plants not comprising the at least one non-natural mutation (e.g., as compared to an isogenic wild type plant not comprising the mutation). A growing area can be any area in which a plurality of plants can be planted together, including, but not limited to, a field (e.g., a cultivated field, an agricultural field), a growth chamber, a greenhouse, a recreational area, a lawn, and/or a roadside, and the like.

In some embodiments, a method of producing/breeding a transgene-free edited plant is provided, the method comprising: crossing a plant of the present invention (e.g., a plant comprising a mutation in a CKX gene and having improved yield traits, e.g., increased planting density, increased pod number, increased seed number (e.g., grain number), and/or increased seed weight (e.g., grain weight)) with a transgene free plant, thereby introducing the at least one non-natural mutation into the plant that is transgene-free (e.g., into progeny plants); and selecting a progeny plant that comprises the at least one non-natural mutation and is transgene-free, thereby producing a transgene free edited (e.g., base edited) plant.

In some embodiments, a method for editing a specific site in the genome of a plant cell is provided, the method comprising cleaving, in a site-specific manner, a target site within an endogenous Cytokinin Oxidase/Dehydrogenase (CKX) gene in the plant cell, wherein the endogenous CKX gene (a) comprises a sequence having at least 80% sequence identity to any one of the nucleotide sequences of SEQ ID NOs:72, 73, 75, 76, 78, 79, 81, 82, 84, 87, 88, or 91; (b) comprises a region having at least 80% sequence identity to any one of the nucleotide sequences of SEQ ID NOs:93-98; and/or (c) encodes a polypeptide having at least 80% sequence identity to any one of the amino acid sequences of SEQ ID NOs:74, 77, 80, 83, 89, or 92, thereby generating an edit in the endogenous CKX gene of the plant cell and producing a plant cell comprising an edit in the endogenous CKX gene. In some embodiments, a plant may be regenerated from the plant cell comprising the edit in the endogenous CKX gene to produce a plant comprising the edit in its genome (i.e., in its endogenous CKX gene). A plant comprising the edit in an endogenous CKX gene can exhibit improved yield traits compared to a control plant that does not comprise the edit in the endogenous CKX gene. In some embodiments, the endogenous CKX gene is a CKX1 gene, a CKX2 gene, a CKX3 gene, a CKX4 gene, a CKX5 gene, and/or a CKX6 gene, or any combination thereof. In some embodiments, a plant comprising the edit in the endogenous CKX gene comprises the edit in at least two (e.g., 2, 3, 4, 5, or 6) different endogenous CKX genes, in any combination (e.g., any combination of at least two of CKX1, CKX2, CKX3, CKX4, CKX5 or CKX6), optionally, wherein the edit is (a) in an endogenous CKX1 gene, an endogenous CKX2 gene, and an endogenous CKX3 gene; (b) in an endogenous CKX1 gene, an endogenous CKX3, an endogenous CKX5 gene, and an endogenous CKX6 gene; or (c) in an endogenous CKX1 gene, an endogenous CKX2 gene, an endogenous CKX3 gene, and an endogenous CKX4 gene.

In some embodiments, the edit results in a non-natural mutation, optionally wherein the non-natural mutation is a point mutation. In some embodiments, the edit produces at least one non-natural mutation that is a base insertion and/or a base deletion, optionally wherein the base deletion is a truncation that results in a C-terminal truncation of at least about 1 amino acid residue to about 540 amino acid residues (e.g., about 1, 2, 3, 4, 5, 6, 7, 8, 9, 10, 11, 12, 13, 14, 15, 20, 25, 30, 35, 40, 50, 60, 70, 80, 90, 100, 110, 120, 130, 140, 150, 160, 170, 180, 190, 191, 192, 193, 194, 195, 200, 210, 220, 225, 230, 240, 250, 275, 300, 325, 350, 400, 410, 420, 430, 435, 436, 437, 438, 439, 440, 450, 455, 460, 465, 470, 475, 476, 477, 478, 479, 480, 485, 486, 487, 488, 489, 490, 495, 500, 505, 510, 515, 520, 521, 523, 524, 525, 526, 527, 528, 529, 530, 531, 532, 534, 535, 536, 537, 538, 539, or 540 amino acid residue(s)) from the C-terminus of a CKX polypeptide, the CKX polypeptide having at least 80% sequence identity to any one of the amino acid sequences of SEQ ID NOs:74, 77, 80, 83, 89, or 92.

In some embodiments, a base deletion can result in a 3′ end truncation of the CKX gene from: (a) about nucleotide 1884, 1885, 1890, 1895, 1900, 1950, 2000, or 2050 to about nucleotide 7399 of the nucleotide sequence of SEQ ID NO:72 (CKX1) or about nucleotide 28, 29, 30, 35, 40, 45, 50, 60, 70, 80, 90, 100, 120, 140, 160, 180, 190, 200, 204, 205, 210, 215, or 220 to about nucleotide 1632 of the nucleotide sequence of SEQ ID NO:73 (CKX1); (b) about nucleotide 803, 804, 805, 810, 820, 830, 840, 850, 860, 870, 880, 890, 900, 910, 920, 930, 940, or 950 to about nucleotide 5917 of the nucleotide sequence of SEQ ID NO:75 (CKX2) or about nucleotide 38, 39, 40, 45, 50, 60, 70, 80, 90, 100, 120, 160, or 180 to about nucleotide 1647 of the nucleotide sequence of SEQ ID NO:76 (CKX2); (c) about nucleotide 692, 693, 694, 695, 700, 710, 720, 730, 740, 750, 760, 770, 780, 790, 800, 810 or 820 to about nucleotide 5768 of the nucleotide sequence of SEQ ID NO:78 (CKX3) or about nucleotide 35, 40, 50, 60, 70, 80, 90, 100, 110, 120, 130, 140, 150, or 160 to about nucleotide 1608 of the nucleotide sequence of SEQ ID NO:79 (CKX3); (d) about nucleotide 1540, 1550, 1560, 1570, 1580, 1590, 1600, 1610, 1620, 1630, 1640, 1650, 1660, 1670, or 1680 to about nucleotide 9725 of the nucleotide sequence of SEQ ID NO:81 (CKX4) or about nucleotide 2, 3, 4, 5, 10, 20, 30, 40, 50, 60, 70, 80, 90, 100, 110, 120, 130, 140, 150 to about nucleotide 1575 of the nucleotide sequence of SEQ ID NO:82 (CKX4); (e) about nucleotide 690, 700, 710, 720, 730, 740, 750, 780 or 790 to about nucleotide 3661 of the nucleotide sequence of SEQ ID NO:84 (CKX5), or about nucleotide 43, 44, 45, 50, 60, 70, 80, 90, 100, 110, 120, 130 or 140 to about nucleotide 1605 of the nucleotide sequence of SEQ ID NO:91 (CKX5); and/or (f) about nucleotide 1562, 1563, 1564, 1565, 1570, 1580, 1590, 1600, 1620, 1640, 1660, 1680, or 1700 to about nucleotide 8277 of the nucleotide sequence of SEQ ID NO:87 (CKX6) or about nucleotide 31, 32, 33, 34, 35, 40, 50, 60, 70, 80, 90, 100, 110, 120, 130, 140, 150, 160 or 170 to about nucleotide 1494 (CKX6) of the nucleotide sequence of SEQ ID NO:88 (CKX6).

In some embodiments, the method of editing produces a non-natural mutation that is a null allele and/or a dominant negative mutation.

In some embodiments, a method for making a plant is provided, the method comprising: (a) contacting a population of plant cells comprising at least one endogenous Cytokinin Oxidase/Dehydrogenase (CKX) gene with a nuclease targeted to the endogenous CKX gene, wherein the nuclease is linked to a nucleic acid binding domain (e.g., an editing system) that binds to a target site in the at least one endogenous CKX gene, wherein the at least one endogenous CKX gene: (i) comprises a sequence having at least 80% sequence identity to any one of the nucleotide sequences of SEQ ID NOs:72, 73, 75, 76, 78, 79, 81, 82, 84, 87, 88, or 91; (ii) comprises a region having at least 80% sequence identity to any one of the nucleotide sequences of SEQ ID NOs:93-98; and/or (iii) encodes a polypeptide having at least 80% identity to any one of the amino acid sequences of SEQ ID NOs: 74, 77, 80, 83, 89, or 92; (b) selecting from the population a plant cell that comprises a mutation in the at least one endogenous CKX gene, wherein the mutation is a substitution and/or a deletion; and (c) growing the selected plant cell into a plant comprising the mutation in the at least one endogenous CKX gene. In some embodiments, the deletion results in a null allele of the endogenous CKX gene; and growing the selected plant cell provides a plant comprising the null allele of the endogenous CKX gene.

In some embodiments, a method for improving yield traits in a plant or part thereof is provided, the method comprising (a) contacting a plant cell comprising an endogenous Cytokinin Oxidase/Dehydrogenase (CKX) gene with a nuclease targeting the endogenous CKX gene, wherein the nuclease is linked to a nucleic acid binding domain that binds to a target site in the endogenous CKX gene, wherein the endogenous CKX gene: (i) comprises a sequence having at least 80% sequence identity to any one of the nucleotide sequences of SEQ ID NOs:72, 73, 75, 76, 78, 79, 81, 82, 84, 87, 88, or 91; (ii) comprises a region having at least 80% sequence identity to any one of the nucleotide sequences of SEQ ID NOs:93-98; and/or (iii) encodes a polypeptide having at least 80% identity to any one of the amino acid sequences of SEQ ID NOs: 74, 77, 80, 83, 89, or 92; and (b) growing the plant cell into a plant comprising the mutation in the endogenous CKX gene, thereby improving yield traits (e.g., increased seed number, increased seed size; increased pod number; or increased yield or improved yield traits as a result of being able to increase planting density) in the plant or part thereof.

In some embodiments, a method for producing a plant or part thereof comprising at least one cell (e.g., one or more cells) having a mutation in an endogenous Cytokinin Oxidase/Dehydrogenase (CKX) gene, the method comprising contacting a target site in the endogenous CKX gene in the plant or plant part with a nuclease comprising a cleavage domain and a nucleic acid binding domain, wherein the nucleic acid binding domain of the nuclease binds to a target site in the endogenous CKX gene, the endogenous CKX gene: (a) comprising a sequence having at least 80% sequence identity to any one of the nucleotide sequences of SEQ ID NOs:72, 73, 75, 76, 78, 79, 81, 82, 84, 87, 88, or 91; (b) comprising a region having at least 80% sequence identity to any one of the nucleotide sequences of SEQ ID NOs:93-98; and/or (c) encoding a polypeptide having at least 80% identity to any one of the amino acid sequences of SEQ ID NOs: 74, 77, 80, 83, 89, or 92, thereby producing the plant or part thereof comprising at least one cell having a mutation in the endogenous CKX gene.

In some embodiments, a method of producing a plant or part thereof comprising a mutation in an endogenous Cytokinin Oxidase/Dehydrogenase (CKX) gene and improved yield traits, the method comprising contacting a target site in an endogenous CKX gene in the plant or plant part with a nuclease comprising a cleavage domain and a nucleic acid binding domain, wherein the nucleic acid binding domain binds to a target site in the endogenous CKX gene, the endogenous CKX gene: (a) comprising a sequence having at least 80% sequence identity to any one of the nucleotide sequences of SEQ ID NOs:72, 73, 75, 76, 78, 79, 81, 82, 84, 87, 88, or 91; (b) comprising a region having at least 80% sequence identity to any one of the nucleotide sequences of SEQ ID NOs:93-98; and/or (c) encoding a polypeptide having at least 80% identity to any one of the amino acid sequences of SEQ ID NOs: 74, 77, 80, 83, 89, or 92, thereby producing the plant or part thereof comprising a mutation in the endogenous CKX gene and exhibiting improved yield traits.

In some embodiments, a nuclease contacting a plant cell, a population of plant cells and/or a target site cleaves an endogenous CKX gene, thereby introducing a mutation into the endogenous CKX gene. A nuclease useful with the invention may be any nuclease that can be utilized to edit/modify a target nucleic acid. Such nucleases include, but are not limited to, a zinc finger nuclease, transcription activator-like effector nucleases (TALEN), endonuclease (e.g., Fok1) and/or a CRISPR-Cas effector protein. Likewise, a nucleic acid binding domain useful with the invention may be any DNA binding domain or RNA binding domain that can be utilized to edit/modify a target nucleic acid. Such nucleic acid binding domains include, but are not limited to, a zinc finger, transcription activator-like DNA binding domain (TAL), an argonaute and/or a CRISPR-Cas effector DNA binding domain.

In some embodiments, a method of editing an endogenous CKX gene in a plant or plant part is provided, the method comprising contacting a target site in CKX gene in the plant or plant part with a cytosine base editing system comprising a cytosine deaminase and a nucleic acid binding domain that binds to a target site in the CKX gene, wherein the CKX gene comprises a sequence having at least 80% sequence identity to any one of the nucleotide sequences of SEQ ID NOs:72, 73, 75, 76, 78, 79, 81, 82, 84, 87, 88, or 91, comprises a region having at least 80% sequence identity to any one of the nucleotide sequences of SEQ ID NOs:93-98 and/or encodes a polypeptide having at least 80% identity to any one of the amino acid sequences of SEQ ID NOs: 74, 77, 80, 83, 89, or 92, thereby producing the plant or part thereof comprising an endogenous CKX gene having a mutation resulting from the contact with the cytosine base editing system, and optionally wherein the plant exhibits improved yield traits.

In some embodiments, a method of editing an endogenous CKX gene in a plant or plant part is provided, the method comprising contacting a target site in CKX gene in the plant or plant part with an adenosine base editing system comprising an adenosine deaminase and a nucleic acid binding domain that binds to a target site in the CKX gene, wherein the CKX gene comprises a sequence having at least 80% sequence identity to any one of the nucleotide sequences of SEQ ID NOs:72, 73, 75, 76, 78, 79, 81, 82, 84, 87, 88, or 91, comprises a region having at least 80% sequence identity to any one of the nucleotide sequences of SEQ ID NOs:93-98 and/or encodes a polypeptide having at least 80% identity to any one of the amino acid sequences of SEQ ID NOs: 74, 77, 80, 83, 89, or 92, thereby producing the plant or part thereof comprising an endogenous CKX gene having a mutation resulting from the contact with the adenosine base editing system, and optionally wherein the plant exhibits improved yield traits.

In some embodiments, a method of detecting a mutant CKX gene (a mutation in an endogenous CKX gene) is provided, the method comprising detecting in the genome of a plant a mutation in an endogenous CKX nucleic acid that encodes an amino acid sequence of, for example, SEQ ID NOs: 74, 77, 80, 83, 89, or 92, which mutation results in a substitution in an amino acid residue of the encoded polypeptide sequence or a deletion of a portion (e.g., at least one residue or 2, 3, 4, 5, 6, 7, 8, 9, 10, 20, 30, 40, 50, 60, 70, 80, 90, 100, 200, 300, 400, or 500 or more consecutive residues) of the encoded polypeptide sequence.

In some embodiments, a method of detecting a mutant CKX gene (a mutation in an endogenous CKX gene) is provided, the method comprising detecting in the genome of a plant a mutation in any one of the nucleotide sequences of, for example, SEQ ID NOs: 72, 73, 75, 76, 78, 79, 81, 82, 84, 87, 88, or 91, optionally wherein the mutation is an insertion, a deletion or substation) of at least one nucleotide (e.g., a deletion of at least 1, 2, 3, 4, 5, 6, 7, 8, 9 10, 20, 30, 40, 50, 60, 70, 80, 90, 100, 200, 300, 400, 500, 1000, 1500, 2000, 2500, 3000, 4000, 5000, or 6000 or more consecutive bases).

In some embodiments, the present invention provides a method of detecting a mutation in an endogenous CKX gene, comprising detecting in the genome of a plant a mutated CKX gene produced as described herein.

In some embodiments, the present invention provides a method of producing a plant comprising a mutation in an endogenous CKX gene and at least one (e.g., one or more) polynucleotide of interest, the method comprising crossing a plant of the invention comprising at least one mutation (e.g., one or more mutations) in an endogenous CKX gene (a first plant) with a second plant that comprises the at least one polynucleotide of interest to produce progeny plants; and selecting progeny plants comprising at least one mutation in the CKX gene and the at least one polynucleotide of interest, thereby producing the plant comprising a mutation in an endogenous CKX gene and at least one polynucleotide of interest.

Further provided is a method of producing a plant comprising a mutation in an endogenous CKX gene and at least one polynucleotide of interest, the method comprising introducing at least one polynucleotide of interest into a plant of the present invention comprising at least one mutation (e.g., one or more mutations) in a CKX gene, thereby producing a plant comprising at least one mutation in a CKX gene and at least one polynucleotide of interest.

A polynucleotide of interest may be any polynucleotide that can confer a desirable phenotype or otherwise modify the phenotype or genotype of a plant. In some embodiments, a polynucleotide of interest may be polynucleotide that confers herbicide tolerance, insect resistance, disease resistance, improved yield traits, increased nutrient use efficiency and/or abiotic stress resistance.

A CKX gene useful with this invention includes any CKX gene that produces a polypeptide that is capable of regulating the cytokinin balance between active cytokinins and in active cytokinins in a plant or part thereof (optionally increasing the active cytokinins over the inactive cytokinins) and in which a mutation as described herein can confer improved yield traits in a plant or part thereof comprising the mutation. In some embodiments, the CKX gene is a CKX1 gene, a CKX2 gene, a CKX3 gene, a CKX4 gene, a CKX5 gene, and/or a CKX6 gene. In some embodiments, at least one non-natural mutation (e.g., one or more non-natural mutations) comprises a mutation in two or more CKX genes (e.g., 2, 3, 4, 5, or 6 CKX genes), e.g., a mutation in two or more of a CKX1 gene, a CKX2 gene, a CKX3 gene, a CKX4 gene, a CKX5 gene, and/or a CKX6 gene, in any combination.

In some embodiments, the mutation in an endogenous CKX gene may be a non-natural mutation. In some embodiments, at least one non-natural mutation (e.g., one or more non-natural mutations) can be a mutation in at least three (e.g., three or more, e.g., 3, 4, 5, or 6) of the endogenous CKX genes of CKX1, CKX2, CKX3, CKX4, CKX5 and/or CKX6 gene, in any combination. In some embodiments, a plant or plant part thereof comprising at least one non-natural mutation in at least one endogenous CKX gene (e.g., one or more endogenous CKX genes) encoding a CKX protein comprises a mutation (a) in an endogenous CKX1 gene, an endogenous CKX2 gene, and an endogenous CKX3 gene; (b) in an endogenous CKX1 gene, an endogenous CKX3, an endogenous CKX5 gene, and an endogenous CKX6 gene; or (c) in an endogenous CKX1 gene, an endogenous CKX2 gene, an endogenous CKX3 gene, and an endogenous CKX4 gene. In some embodiments, a plant comprising at least one non-natural mutation in at least one endogenous CKX gene encoding a CKX protein exhibits improved yield traits compared to an isogenic plant that does not comprise the mutation.

In some embodiments, the non-natural mutation may be any mutation in an endogenous CKX gene that results in improved yield traits when comprised in a plant. In some embodiments, the at least one non-natural mutation in an endogenous CKX gene (e.g., one or more endogenous CKX genes) can be a point mutation, optionally a base substitution, a base insertion and/or a base deletion. In some embodiments, the at least one non-natural mutation in an endogenous CKX gene is a null mutation and/or a dominant negative mutation. In some embodiments, the at least one non-natural mutation in an endogenous CKX gene in a plant may be a substitution, a deletion and/or an insertion that results in a plant exhibiting improved yield traits. In some embodiments, the at least one non-natural mutation in an endogenous CKX gene in a plant may be a substitution, a deletion and/or an insertion that results in a dominant negative mutation or a null mutation and a plant having improved yield traits. In some embodiments, the at least one non-natural mutation may be a base substitution to an A, a T, a G, or a C. In some embodiments, the at least one non-natural mutation may be a deletion of a portion or the entire CKX gene or CKX protein (e.g., a CKX1, CKX2, CKX3, CKX4, CKX5 or CKX6 gene or polypeptide).

In some embodiments, the present invention provides a guide nucleic acid (e.g., gRNA, gDNA, crRNA, crDNA) that binds to a target site in a Cytokinin Oxidase/Dehydrogenase (CKX) gene, the CKX gene: (a) comprising a sequence having at least 80% sequence identity to any one of the nucleotide sequences of SEQ ID NOs:72, 73, 75, 76, 78, 79, 81, 82, 84, 87, 88, or 91; (b) comprising a region having at least 80% sequence identity to any one of the nucleotide sequences of SEQ ID NOs:93-98; and/or (c) encoding a polypeptide having at least 80% identity to any one of the amino acid sequences of SEQ ID NOs: 74, 77, 80, 83, 89, or 92.

Example spacer sequences useful with a guide of this invention may comprise complementarity to a fragment or portion of a nucleotide sequence having at least 80% sequence identity to any one of the nucleotide sequences of SEQ ID NOs:72, 73, 75, 76, 78, 79, 81, 82, 84, 87, 88, or 91; or a fragment or portion of a nucleotide sequence encoding a polypeptide comprising a sequence having at least 80% sequence identity to any one of the amino acid sequences SEQ ID NOs:93-98.

In some embodiments, a target nucleic acid is an endogenous CKX gene that is capable of regulating the cytokinin balance between active cytokinins and in active cytokinins in a plant, optionally increasing the active cytokinins over the inactive cytokinins. In some embodiments, a target site in a target nucleic acid may comprise a sequence having at least 80% sequence identity to a region, portion or fragment of SEQ ID NOs:72, 73, 75, 76, 78, 79, 81, 82, 84, 87, 88, 91 or 93-98, or a target site in a target nucleic acid may encode a region of an amino acid sequence having at least 80% sequence identity to SEQ ID NOs:74, 77, 80, 83, 89, or 92.

In some embodiments, a guide nucleic acid comprises a spacer having the nucleotide sequence of any one of SEQ ID NOs:99-113. In some embodiments, a CKX polypeptide may be a CKX1, CKX2, CKX3, CKX4, CKX5 and/or a CKX6 polypeptide.

In some embodiments, a system is provided that comprises a guide nucleic acid of the present invention and a CRISPR-Cas effector protein that associates with the guide nucleic acid. In some embodiments, the system may further comprise a tracr nucleic acid that associates with the guide nucleic acid and a CRISPR-Cas effector protein, optionally wherein the tracr nucleic acid and the guide nucleic acid are covalently linked.

As used herein, “a CRISPR-Cas effector protein in association with a guide nucleic acid” refers to the complex that is formed between a CRISPR-Cas effector protein and a guide nucleic acid in order to direct the CRISPR-Cas effector protein to a target site in a gene.

In some embodiments, a gene editing system is provided, the gene editing system comprising a CRISPR-Cas effector protein in association with a guide nucleic acid, wherein the guide nucleic acid comprises a spacer sequence that binds to a CKX gene. In some embodiments, a CKX gene useful with the gene editing system (a) comprises a sequence having at least 80% sequence identity to any one of the nucleotide sequences of SEQ ID NOs:72, 73, 75, 76, 78, 79, 81, 82, 84, 87, 88, or 91; (b) comprises a region having at least 80% sequence identity to any one of the nucleotide sequences of SEQ ID NOs:93-98; and/or (c) encodes a polypeptide having at least 80% sequence identity to any one of the amino acid sequences of SEQ ID NOs:74, 77, 80, 83, 89, or 92. In some embodiments, a CKX polypeptide may be a CKX1, CKX2, CKX3, CKX4, CKX5 and/or a CKX6 polypeptide.

In some embodiments, the guide nucleic acid of a gene editing system can comprise a spacer sequence that has complementarity to a region, portion or fragment of a nucleotide sequence having at least 80% sequence identity to any one of the nucleotide sequences of SEQ ID NOs:72, 73, 75, 76, 78, 79, 81, 82, 84, 87, 88, 91 or 93-98, or may encode a region, portion or fragment of an amino acid sequence having at least 80% sequence identity to SEQ ID NOs:74, 77, 80, 83, 89, or 92. In some embodiments, a gene editing system may further comprise a tracr nucleic acid that associates with the guide nucleic acid and a CRISPR-Cas effector protein, optionally wherein the tracr nucleic acid and the guide nucleic acid are covalently linked.

In some embodiments, a guide nucleic acid is provided that binds to a target nucleic acid in an endogenous Cytokinin Oxidase/Dehydrogenase (CKX) gene having the gene identification number (gene ID) of Glyma15g18560, Glyma09g07360, Glyma17g06220, Glyma04g03130, Glyma09g35950 and/or Glyma09g07190.

The present invention further provides a complex comprising a CRISPR-Cas effector protein comprising a cleavage domain and a guide nucleic acid, wherein the guide nucleic acid binds to a target site in an endogenous Cytokinin Oxidase/Dehydrogenase (CKX) gene, wherein the CKX gene (a) comprises a sequence having at least 80% sequence identity to any one of the nucleotide sequences of SEQ ID NOs:72, 73, 75, 76, 78, 79, 81, 82, 84, 87, 88, or 91; (b) comprises a region having at least 80% sequence identity to any one of the nucleotide sequences of SEQ ID NOs:93-98; and/or (c) encodes a polypeptide having at least 80% sequence identity to any one of the amino acid sequences of SEQ ID NOs:74, 77, 80, 83, 89, or 92, wherein the cleavage domain cleaves a target strand in the CKX gene. In some embodiments, a CKX gene may be a CKX1, CKX2, CKX3, CKX4, CKX5 and/or a CKX6 gene.

Also provided herein are expression cassettes comprising (a) a polynucleotide encoding CRISPR-Cas effector protein comprising a cleavage domain and (b) a guide nucleic acid that binds to a target site in an endogenous Cytokinin Oxidase/Dehydrogenase (CKX) gene, wherein the guide nucleic acid comprises a spacer sequence that is complementary to and binds to a portion of the endogenous CKX gene, the endogenous CKX gene having at least 80% sequence identity to any one of the nucleotide sequences of SEQ ID NOs:72, 73, 75, 76, 78, 79, 81, 82, 84, 87, 88, or 91 or encoding a sequence having at least 80% sequence identity to any one of the amino acid sequences of SEQ ID NOs:74, 77, 80, 83, 89, or 92, optionally wherein the spacer sequence is complementary to and binds to a portion of the endogenous CKX gene having at least 80% sequence identity to any one of the nucleotide sequences of SEQ ID NOs:93-98. In some embodiments, a CKX gene may be a CKX1, CKX2, CKX3, CKX4, CKX5 and/or a CKX6 gene.

An editing system useful with this invention can be any site-specific (sequence-specific) genome editing system now known or later developed, which system can introduce mutations in target specific manner. For example, an editing system (e.g., site- or sequence-specific editing system) can include, but is not limited to, a CRISPR-Cas editing system, a meganuclease editing system, a zinc finger nuclease (ZFN) editing system, a transcription activator-like effector nuclease (TALEN) editing system, a base editing system and/or a prime editing system, each of which can comprise one or more polypeptides and/or one or more polynucleotides that when expressed as a system in a cell can modify (mutate) a target nucleic acid in a sequence specific manner. In some embodiments, an editing system (e.g., site- or sequence-specific editing system) can comprise one or more polynucleotides and/or one or more polypeptides, including but not limited to a nucleic acid binding domain (DNA binding domain), a nuclease, and/or other polypeptide, and/or a polynucleotide, and/or a guide nucleic acid (comprising a spacer having substantial complementarity or full complementarity to a target site).

In some embodiments, an editing system can comprise one or more sequence-specific nucleic acid binding domains (DNA binding domains) that can be from, for example, a polynucleotide-guided endonuclease, a CRISPR-Cas endonuclease (e.g., CRISPR-Cas effector protein), a zinc finger nuclease, a transcription activator-like effector nuclease (TALEN) and/or an Argonaute protein. In some embodiments, an editing system can comprise one or more cleavage domains (e.g., nucleases) including, but not limited to, an endonuclease (e.g., Fok1), a polynucleotide-guided endonuclease, a CRISPR-Cas endonuclease (e.g., CRISPR-Cas effector protein), a zinc finger nuclease, and/or a transcription activator-like effector nuclease (TALEN). In some embodiments, an editing system can comprise one or more polypeptides that include, but are not limited to, a deaminase (e.g., a cytosine deaminase, an adenine deaminase), a reverse transcriptase, a Dna2 polypeptide, and/or a 5′ flap endonuclease (FEN). In some embodiments, an editing system can comprise one or more polynucleotides, including, but is not limited to, a CRISPR array (CRISPR guide) nucleic acid, extended guide nucleic acid, and/or a reverse transcriptase template.

In some embodiments, a method of modifying or editing a CKX polypeptide may comprise contacting a target nucleic acid (e.g., a nucleic acid encoding a CKX polypeptide) with a base-editing fusion protein (e.g., a sequence specific DNA binding protein (e.g., a CRISPR-Cas effector protein or domain) fused to a deaminase domain (e.g., an adenine deaminase and/or a cytosine deaminase) and a guide nucleic acid, wherein the guide nucleic acid is capable of guiding/targeting the base editing fusion protein to the target nucleic acid, thereby editing a locus within the target nucleic acid. In some embodiments, a base editing fusion protein and guide nucleic acid may be comprised in one or more expression cassettes. In some embodiments, the target nucleic acid may be contacted with a base editing fusion protein and an expression cassette comprising a guide nucleic acid. In some embodiments, the sequence-specific DNA binding fusion proteins and guides may be provided as ribonucleoproteins (RNPs). In some embodiments, a cell may be contacted with more than one base-editing fusion protein and/or one or more guide nucleic acids that may target one or more target nucleic acids in the cell.

In some embodiments, a method of modifying or editing a CKX gene may comprise contacting a target nucleic acid (e.g., a nucleic acid encoding a CKX polypeptide) with a sequence-specific DNA binding fusion protein (e.g., a sequence-specific DNA binding protein (e.g., a CRISPR-Cas effector protein or domain) fused to a peptide tag, a deaminase fusion protein comprising a deaminase domain (e.g., an adenine deaminase and/or a cytosine deaminase) fused to an affinity polypeptide that is capable of binding to the peptide tag, and a guide nucleic acid, wherein the guide nucleic acid is capable of guiding/targeting the sequence-specific DNA binding fusion protein to the target nucleic acid and the sequence-specific DNA binding fusion protein is capable of recruiting the deaminase fusion protein to the target nucleic acid via the peptide tag-affinity polypeptide interaction, thereby editing a locus within the target nucleic acid. In some embodiments, the sequence-specific DNA binding fusion protein may be fused to the affinity polypeptide that binds the peptide tag and the deaminase may be fuse to the peptide tag, thereby recruiting the deaminase to the sequence-specific DNA binding fusion protein and to the target nucleic acid. In some embodiments, the sequence-specific binding fusion protein, deaminase fusion protein, and guide nucleic acid may be comprised in one or more expression cassettes. In some embodiments, the target nucleic acid may be contacted with a sequence-specific binding fusion protein, deaminase fusion protein, and an expression cassette comprising a guide nucleic acid. In some embodiments, the sequence-specific DNA binding fusion proteins, deaminase fusion proteins and guides may be provided as ribonucleoproteins (RNPs).

In some embodiments, methods such as prime editing may be used to generate a mutation in an endogenous CKX gene. In prime editing, RNA-dependent DNA polymerase (reverse transcriptase, RT) and reverse transcriptase templates (RT template) are used in combination with sequence specific nucleic acid binding domains that confer the ability to recognize and bind the target in a sequence-specific manner, and which can also cause a nick of the PAM-containing strand within the target. The sequence specific nucleic acid binding domain may be a CRISPR-Cas effector protein and in this case, the CRISPR array or guide RNA may be an extended guide that comprises an extended portion comprising a primer binding site (PSB) and the edit to be incorporated into the genome (the template). Similar to base editing, prime editing can take advantageous of the various methods of recruiting proteins for use in the editing to the target site, such methods including both non-covalent and covalent interactions between the proteins and nucleic acids used in the selected process of genome editing.

In some embodiments, the mutation or modification of a CKX gene may be an insertion, a deletion and/or a point mutation in that produces a CKX polypeptide having, for example, a C-terminal truncation (e.g., a mutated CKX polypeptide) or the mutation of a CKX gene may result in no CKX polypeptide. In some embodiments, a plant comprising an endogenous CKX gene having a mutation as described herein (e.g., at least one mutation (e.g., one or more mutations) in an endogenous CKX gene, optionally wherein no CKX polypeptide is produced or the CKX polypeptide that is produced is truncated) may comprise improved yield traits compared to a control plant that does not comprise the at least one non-natural mutation in an endogenous CKX gene.

In some embodiments, a plant part may be a cell. In some embodiments, the plant or plant part thereof may be any plant or part thereof as described herein. In some embodiments, a plant useful with this invention may be corn, soybean, canola, wheat, rice, cotton, sugarcane, sugar beet, barley, oats, alfalfa, sunflower, safflower, oil palm. sesame, coconut, tobacco, potato, sweet potato, cassava, coffee, apple, plum, apricot, peach, cherry, pear, fig, banana, citrus, cocoa, avocado, olive, almond, walnut, strawberry, watermelon, pepper, grape, tomato, cucumber, or a Brassica spp. In some embodiments, the plant may be a soybean plant and the plant part, including a cell, may be from a soybean plant.

In some embodiments, a mutation that is introduced into an endogenous CKX gene polypeptide is a non-natural mutation. In some embodiments, a mutation that is introduced into an endogenous CKX gene may be a substitution, an insertion and/or a deletion of one or more nucleotides as described herein. In some embodiments, a mutation that is introduced into an endogenous CKX gene may be a deletion, optionally a deletion of all or a portion of the CKX gene, e.g., a 3′ truncation of the gene resulting in a CKX polypeptide with a C-terminal truncation or no CKX polypeptide. In some embodiments, a mutation in an endogenous CKX gene may result in altered expression (e.g., increased or decreased expression) of the gene as compared to a CKX gene, and therefore an altered amount of the CKX polypeptide compared to the corresponding CKX gene (e.g., the CKX gene not modified as described herein). In some embodiments, a mutation in the promoter (e.g., promoter bashing) of an endogenous CKX gene may result in modified (increased/decreased) expression of the CKX gene, and therefore an increased amount of the CKX polypeptide. In some embodiments, a mutation in an endogenous CKX gene may result in reduced expression of the gene as compared to a CKX gene, and therefore a reduced amount of the CKX polypeptide that is a null or inactive polypeptide. In some embodiments, a mutated CKX gene as described herein may have the same expression level as the CKX gene, but the mutated CKX gene produces a null or inactive CKX polypeptide.

In some embodiments, a CKX gene may be a CKX1 gene, CKX2 gene, CKX3 gene, CKX4 gene, CKX5 gene or CKX6 gene. In some embodiments, a CKX polypeptide may be a CKX1 polypeptide, CKX2 polypeptide, CKX3 polypeptide, CKX4 polypeptide, CKX5 polypeptide or CKX6 polypeptide. In some embodiments, a plant or part thereof may comprise a mutation in two or more endogenous CKX genes. For example, a plant or part thereof may comprise a non-natural mutation in (a) in a CKX1 gene, a CKX2 gene, and a CKX3 gene; (b) in a CKX1 gene, a CKX3, a CKX5 gene, and a CKX6 gene; or (c) in a CKX1 gene, a CKX2 gene, a CKX3 gene, and a CKX4 gene. Further combinations of the CKX genes comprising non-natural mutations as described herein are contemplated to be useful for producing a plant exhibiting improvements in yield components.

In some embodiments, a sequence-specific nucleic acid binding domain (DNA binding domains) of an editing system useful with this invention can be from, for example, a polynucleotide-guided endonuclease, a CRISPR-Cas endonuclease (e.g., CRISPR-Cas effector protein), a zinc finger nuclease, a transcription activator-like effector nuclease (TALEN) and/or an Argonaute protein.

In some embodiments, a sequence-specific nucleic acid binding domain may be a CRISPR-Cas effector protein, optionally wherein the CRISPR-Cas effector protein may be from a Type I CRISPR-Cas system, a Type II CRISPR-Cas system, a Type III CRISPR-Cas system, a Type IV CRISPR-Cas system, Type V CRISPR-Cas system, or a Type VI CRISPR-Cas system. In some embodiments, a CRISPR-Cas effector protein of the invention may be from a Type II CRISPR-Cas system or a Type V CRISPR-Cas system. In some embodiments, a CRISPR-Cas effector protein may be Type II CRISPR-Cas effector protein, for example, a Cas9 effector protein. In some embodiments, a CRISPR-Cas effector protein may be Type V CRISPR-Cas effector protein, for example, a Cas12 effector protein.

As used herein, a “CRISPR-Cas effector protein” is a protein or polypeptide or domain thereof that cleaves or cuts a nucleic acid, binds a nucleic acid (e.g., a target nucleic acid and/or a guide nucleic acid), and/or that identifies, recognizes, or binds a guide nucleic acid as defined herein. In some embodiments, a CRISPR-Cas effector protein may be an enzyme (e.g., a nuclease, endonuclease, nickase, etc.) or portion thereof and/or may function as an enzyme. In some embodiments, a CRISPR-Cas effector protein refers to a CRISPR-Cas nuclease polypeptide or domain thereof that comprises nuclease activity or in which the nuclease activity has been reduced or eliminated, and/or comprises nickase activity or in which the nickase has been reduced or eliminated, and/or comprises single stranded DNA cleavage activity (ss DNAse activity) or in which the ss DNAse activity has been reduced or eliminated, and/or comprises self-processing RNAse activity or in which the self-processing RNAse activity has been reduced or eliminated. A CRISPR-Cas effector protein may bind to a target nucleic acid.

In some embodiments, a CRISPR-Cas effector protein may include, but is not limited to, a Cas9, C2c1, C2c3, Cas12a (also referred to as Cpf1), Cas12b, Cas12c, Cas12d, Cas12e, Cas13a, Cas13b, Cas13c, Cas13d, Cas1, Cas1B, Cas2, Cas3, Cas3′, Cas3″, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4 (dinG), and/or Csf5 nuclease, optionally wherein the CRISPR-Cas effector protein may be a Cas9, Cas12a (Cpf1), Cas12b, Cas12c (C2c3), Cas12d (CasY), Cas12e (CasX), Cas12g, Cas12h, Cas12i, C2c4, C2c5, C2c8, C2c9, C2c10, Cas14a, Cas14b, and/or Cas14c effector protein.

In some embodiments, a CRISPR-Cas effector protein useful with the invention may comprise a mutation in its nuclease active site (e.g., RuvC, HNH, e.g., RuvC site of a Cas12a nuclease domain; e.g., RuvC site and/or HNH site of a Cas9 nuclease domain). A CRISPR-Cas effector protein having a mutation in its nuclease active site, and therefore, no longer comprising nuclease activity, is commonly referred to as “dead,” e.g., dCas. In some embodiments, a CRISPR-Cas effector protein domain or polypeptide having a mutation in its nuclease active site may have impaired activity or reduced activity as compared to the same CRISPR-Cas effector protein without the mutation, e.g., a nickase, e.g, Cas9 nickase, Cas12a nickase.

A CRISPR Cas9 effector protein or CRISPR Cas9 effector domain useful with this invention may be any known or later identified Cas9 nuclease. In some embodiments, a CRISPR Cas9 polypeptide can be a Cas9 polypeptide from, for example, Streptococcus spp. (e.g., S. pyogenes, S. thermophiles), Lactobacillus spp., Bifidobacterium spp., Kandleria spp., Leuconostoc spp., Oenococcus spp., Pediococcus spp., Weissella spp., and/or Olsenella spp. Example Cas9 sequences include, but are not limited to, the amino acid sequences of SEQ ID NOs:59-60 or the polynucleotide sequences of SEQ ID NOs:61-71.

In some embodiments, the CRISPR-Cas effector protein may be a Cas9 polypeptide derived from Streptococcus pyogenes and recognizes the PAM sequence motif NGG, NAG, NGA (Mali et al, Science 2013; 339(6121): 823-826). In some embodiments, the CRISPR-Cas effector protein may be a Cas9 polypeptide derived from Streptococcus thermophiles and recognizes the PAM sequence motif NGGNG and/or NNAGAAW (W=A or T) (See, e.g., Horvath et al, Science, 2010; 327(5962): 167-170, and Deveau et al, J Bacteriol 2008; 190(4): 1390-1400). In some embodiments, the CRISPR-Cas effector protein may be a Cas9 polypeptide derived from Streptococcus mutans and recognizes the PAM sequence motif NGG and/or NAAR (R=A or G) (See, e.g., Deveau et al, J BACTERIOL 2008; 190(4): 1390-1400). In some embodiments, the CRISPR-Cas effector protein may be a Cas9 polypeptide derived from Streptococcus aureus and recognizes the PAM sequence motif NNGRR (R=A or G). In some embodiments, the CRISPR-Cas effector protein may be a Cas9 protein derived from S. aureus, which recognizes the PAM sequence motif N GRRT (R=A or G). In some embodiments, the CRISPR-Cas effector protein may be a Cas9 polypeptide derived from S. aureus, which recognizes the PAM sequence motif N GRRV (R=A or G). In some embodiments, the CRISPR-Cas effector protein may be a Cas9 polypeptide that is derived from Neisseria meningitidis and recognizes the PAM sequence motif N GATT or N GCTT (R=A or G, V=A, G or C) (See, e.g., Hou et ah, PNAS 2013, 1-6). In the aforementioned embodiments, N can be any nucleotide residue, e.g., any of A, G, C or T. In some embodiments, the CRISPR-Cas effector protein may be a Cas13a protein derived from Leptotrichia shahii, which recognizes a protospacer flanking sequence (PFS) (or RNA PAM (rPAM)) sequence motif of a single 3′ A, U, or C, which may be located within the target nucleic acid.

In some embodiments, the CRISPR-Cas effector protein may be derived from Cas12a, which is a Type V Clustered Regularly Interspaced Short Palindromic Repeats (CRISPR)-Cas nuclease (see, e.g., SEQ ID NOs:1-20). Cas12a differs in several respects from the more well-known Type II CRISPR Cas9 nuclease. For example, Cas9 recognizes a G-rich protospacer-adjacent motif (PAM) that is 3′ to its guide RNA (gRNA, sgRNA, crRNA, crDNA, CRISPR array) binding site (protospacer, target nucleic acid, target DNA) (3′-NGG), while Cas12a recognizes a T-rich PAM that is located 5′ to the target nucleic acid (5′-TTN, 5′-TTTN. In fact, the orientations in which Cas9 and Cas12a bind their guide RNAs are very nearly reversed in relation to their N and C termini. Furthermore, Cas12a enzymes use a single guide RNA (gRNA, CRISPR array, crRNA) rather than the dual guide RNA (sgRNA (e.g., crRNA and tracrRNA)) found in natural Cas9 systems, and Cas12a processes its own gRNAs. Additionally, Cas12a nuclease activity produces staggered DNA double stranded breaks instead of blunt ends produced by Cas9 nuclease activity, and Cas12a relies on a single RuvC domain to cleave both DNA strands, whereas Cas9 utilizes an HNH domain and a RuvC domain for cleavage.

A CRISPR Cas12a effector protein/domain useful with this invention may be any known or later identified Cas12a polypeptide (previously known as Cpf1) (see, e.g., U.S. Pat. No. 9,790,490, which is incorporated by reference for its disclosures of Cpf1 (Cas12a) sequences). The term “Cas12a”, “Cas12a polypeptide” or “Cas12a domain” refers to an RNA-guided nuclease comprising a Cas12a polypeptide, or a fragment thereof, which comprises the guide nucleic acid binding domain of Cas12a and/or an active, inactive, or partially active DNA cleavage domain of Cas12a. In some embodiments, a Cas12a useful with the invention may comprise a mutation in the nuclease active site (e.g., RuvC site of the Cas12a domain). A Cas12a domain or Cas12a polypeptide having a mutation in its nuclease active site, and therefore, no longer comprising nuclease activity, is commonly referred to as deadCas12a (e.g., dCas12a). In some embodiments, a Cas12a domain or Cas12a polypeptide having a mutation in its nuclease active site may have impaired activity, e.g., may have nickase activity.

Any deaminase domain/polypeptide useful for base editing may be used with this invention. In some embodiments, the deaminase domain may be a cytosine deaminase domain or an adenine deaminase domain. A cytosine deaminase (or cytidine deaminase) useful with this invention may be any known or later identified cytosine deaminase from any organism (see, e.g., U.S. Pat. No. 10,167,457 and Thuronyi et al. Nat. Biotechnol. 37:1070-1079 (2019), each of which is incorporated by reference herein for its disclosure of cytosine deaminases). Cytosine deaminases can catalyze the hydrolytic deamination of cytidine or deoxycytidine to uridine or deoxyuridine, respectively. Thus, in some embodiments, a deaminase or deaminase domain useful with this invention may be a cytidine deaminase domain, catalyzing the hydrolytic deamination of cytosine to uracil. In some embodiments, a cytosine deaminase may be a variant of a naturally occurring cytosine deaminase, including but not limited to a primate (e.g., a human, monkey, chimpanzee, gorilla), a dog, a cow, a rat or a mouse. Thus, in some embodiments, an cytosine deaminase useful with the invention may be about 70% to about 100% identical to a wild type cytosine deaminase (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical, and any range or value therein, to a naturally occurring cytosine deaminase).

In some embodiments, a cytosine deaminase useful with the invention may be an apolipoprotein B mRNA-editing complex (APOBEC) family deaminase. In some embodiments, the cytosine deaminase may be an APOBEC1 deaminase, an APOBEC2 deaminase, an APOBEC3A deaminase, an APOBEC3B deaminase, an APOBEC3C deaminase, an APOBEC3D deaminase, an APOBEC3F deaminase, an APOBEC3G deaminase, an APOBEC3H deaminase, an APOBEC4 deaminase, a human activation induced deaminase (hAID), an rAPOBEC1, FERNY, and/or a CDA1, optionally a pmCDA1, an atCDA1 (e.g., At2g19570), and evolved versions of the same (e.g., SEQ ID NO:27, SEQ ID NO:28 or SEQ ID NO:29). In some embodiments, the cytosine deaminase may be an APOBEC1 deaminase having the amino acid sequence of SEQ ID NO:23. In some embodiments, the cytosine deaminase may be an APOBEC3A deaminase having the amino acid sequence of SEQ ID NO:24. In some embodiments, the cytosine deaminase may be an CDA1 deaminase, optionally a CDA1 having the amino acid sequence of SEQ ID NO:25. In some embodiments, the cytosine deaminase may be a FERNY deaminase, optionally a FERNY having the amino acid sequence of SEQ ID NO:26. In some embodiments, a cytosine deaminase useful with the invention may be about 70% to about 100% identical (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100% identical) to the amino acid sequence of a naturally occurring cytosine deaminase (e.g., an evolved deaminase). In some embodiments, a cytosine deaminase useful with the invention may be about 70% to about 99.5% identical (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.5% identical) to the amino acid sequence of SEQ ID NO:23, SEQ ID NO:24, SEQ ID NO:25 or SEQ ID NO:26 (e.g., at least 80%, at least 85%, at least 90%, at least 92%, at least 95%, at least 96%, at least 97%, at least 98%, at least 99%, or at least 99.5% identical to the amino acid sequence of SEQ ID NO:23, SEQ ID NO:24, SEQ ID NO:25, SEQ ID NO:26, SEQ ID NO:27, SEQ ID NO:28 or SEQ ID NO:29). In some embodiments, a polynucleotide encoding a cytosine deaminase may be codon optimized for expression in a plant and the codon optimized polypeptide may be about 70% to 99.5% identical to the reference polynucleotide.

In some embodiments, a nucleic acid construct of this invention may further encode an uracil glycosylase inhibitor (UGI) (e.g., uracil-DNA glycosylase inhibitor) polypeptide/domain. Thus, in some embodiments, a nucleic acid construct encoding a CRISPR-Cas effector protein and a cytosine deaminase domain (e.g., encoding a fusion protein comprising a CRISPR-Cas effector protein domain fused to a cytosine deaminase domain, and/or a CRISPR-Cas effector protein domain fused to a peptide tag or to an affinity polypeptide capable of binding a peptide tag and/or a deaminase protein domain fused to a peptide tag or to an affinity polypeptide capable of binding a peptide tag) may further encode a uracil-DNA glycosylase inhibitor (UGI), optionally wherein the UGI may be codon optimized for expression in a plant. In some embodiments, the invention provides fusion proteins comprising a CRISPR-Cas effector polypeptide, a deaminase domain, and a UGI and/or one or more polynucleotides encoding the same, optionally wherein the one or more polynucleotides may be codon optimized for expression in a plant. In some embodiments, the invention provides fusion proteins, wherein a CRISPR-Cas effector polypeptide, a deaminase domain, and a UGI may be fused to any combination of peptide tags and affinity polypeptides as described herein, thereby recruiting the deaminase domain and UGI to the CRISPR-Cas effector polypeptide and a target nucleic acid. In some embodiments, a guide nucleic acid may be linked to a recruiting RNA motif and one or more of the deaminase domain and/or UGI may be fused to an affinity polypeptide that is capable of interacting with the recruiting RNA motif, thereby recruiting the deaminase domain and UGI to a target nucleic acid.

A “uracil glycosylase inhibitor” useful with the invention may be any protein that is capable of inhibiting a uracil-DNA glycosylase base-excision repair enzyme. In some embodiments, a UGI domain comprises a wild type UGI or a fragment thereof. In some embodiments, a UGI domain useful with the invention may be about 70% to about 100% identical (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100% identical and any range or value therein) to the amino acid sequence of a naturally occurring UGI domain. In some embodiments, a UGI domain may comprise the amino acid sequence of SEQ ID NO:41 or a polypeptide having about 70% to about 99.5% sequence identity to the amino acid sequence of SEQ ID NO:41 (e.g., at least 80%, at least 85%, at least 90%, at least 92%, at least 95%, at least 96%, at least 97%, at least 98%, at least 99%, or at least 99.5% identical to the amino acid sequence of SEQ ID NO:41). For example, in some embodiments, a UGI domain may comprise a fragment of the amino acid sequence of SEQ ID NO:41 that is 100% identical to a portion of consecutive nucleotides (e.g., 10, 15, 20, 25, 30, 35, 40, 45, 50, 55, 60, 65, 70, 75, 80 consecutive nucleotides; e.g., about 10, 15, 20, 25, 30, 35, 40, 45, to about 50, 55, 60, 65, 70, 75, 80 consecutive nucleotides) of the amino acid sequence of SEQ ID NO:41. In some embodiments, a UGI domain may be a variant of a known UGI (e.g., SEQ ID NO:41) having about 70% to about 99.5% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% sequence identity, and any range or value therein) to the known UGI. In some embodiments, a polynucleotide encoding a UGI may be codon optimized for expression in a plant (e.g., a plant) and the codon optimized polypeptide may be about 70% to about 99.5% identical to the reference polynucleotide.

An adenine deaminase (or adenosine deaminase) useful with this invention may be any known or later identified adenine deaminase from any organism (see, e.g., U.S. Pat. No. 10,113,163, which is incorporated by reference herein for its disclosure of adenine deaminases). An adenine deaminase can catalyze the hydrolytic deamination of adenine or adenosine. In some embodiments, the adenine deaminase may catalyze the hydrolytic deamination of adenosine or deoxyadenosine to inosine or deoxyinosine, respectively. In some embodiments, the adenosine deaminase may catalyze the hydrolytic deamination of adenine or adenosine in DNA. In some embodiments, an adenine deaminase encoded by a nucleic acid construct of the invention may generate an A→G conversion in the sense (e.g., “+”; template) strand of the target nucleic acid or a T→C conversion in the antisense (e.g., “−”, complementary) strand of the target nucleic acid.

In some embodiments, an adenosine deaminase may be a variant of a naturally occurring adenine deaminase. Thus, in some embodiments, an adenosine deaminase may be about 70% to 100% identical to a wild type adenine deaminase (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical, and any range or value therein, to a naturally occurring adenine deaminase). In some embodiments, the deaminase or deaminase does not occur in nature and may be referred to as an engineered, mutated or evolved adenosine deaminase. Thus, for example, an engineered, mutated or evolved adenine deaminase polypeptide or an adenine deaminase domain may be about 70% to 99.9% identical to a naturally occurring adenine deaminase polypeptide/domain (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 99.9% identical, and any range or value therein, to a naturally occurring adenine deaminase polypeptide or adenine deaminase domain). In some embodiments, the adenosine deaminase may be from a bacterium, (e.g., Escherichia coli, Staphylococcus aureus, Haemophilus influenzae, Caulobacter crescentus, and the like). In some embodiments, a polynucleotide encoding an adenine deaminase polypeptide/domain may be codon optimized for expression in a plant.

In some embodiments, an adenine deaminase domain may be a wild type tRNA-specific adenosine deaminase domain, e.g., a tRNA-specific adenosine deaminase (TadA) and/or a mutated/evolved adenosine deaminase domain, e.g., mutated/evolved tRNA-specific adenosine deaminase domain (TadA*). In some embodiments, a TadA domain may be from E. coli. In some embodiments, the TadA may be modified, e.g., truncated, missing one or more N-terminal and/or C-terminal amino acids relative to a full-length TadA (e.g., 1, 2, 3, 4, 5, 6, 7, 8, 9, 10, 11, 12, 13, 14, 15, 6, 17, 18, 19, or 20 N-terminal and/or C terminal amino acid residues may be missing relative to a full length TadA. In some embodiments, a TadA polypeptide or TadA domain does not comprise an N-terminal methionine. In some embodiments, a wild type E. coli TadA comprises the amino acid sequence of SEQ ID NO:30. In some embodiments, a mutated/evolved E. coli TadA* comprises the amino acid sequence of SEQ ID NOs:31-40 (e.g., SEQ ID NOs:31, 32, 33, 34, 35, 36, 37, 38, 39 or 40). In some embodiments, a polynucleotide encoding a TadA/TadA* may be codon optimized for expression in a plant.

A cytosine deaminase catalyzes cytosine deamination and results in a thymidine (through a uracil intermediate), causing a C to T conversion, or a G to A conversion in the complementary strand in the genome. Thus, in some embodiments, the cytosine deaminase encoded by the polynucleotide of the invention generates a C→T conversion in the sense (e.g., “+”; template) strand of the target nucleic acid or a G→A conversion in antisense (e.g., “−”, complementary) strand of the target nucleic acid.

In some embodiments, the adenine deaminase encoded by the nucleic acid construct of the invention generates an A→G conversion in the sense (e.g., “+”; template) strand of the target nucleic acid or a T→C conversion in the antisense (e.g., “−”, complementary) strand of the target nucleic acid.

The nucleic acid constructs of the invention encoding a base editor comprising a sequence-specific DNA binding protein and a cytosine deaminase polypeptide, and nucleic acid constructs/expression cassettes/vectors encoding the same, may be used in combination with guide nucleic acids for modifying target nucleic acid including, but not limited to, generation of C→T or G→A mutations in a target nucleic acid including, but not limited to, a plasmid sequence; generation of C→T or G→A mutations in a coding sequence to alter an amino acid identity; generation of C→T or G→A mutations in a coding sequence to generate a stop codon; generation of C→T or G→A mutations in a coding sequence to disrupt a start codon; generation of point mutations in genomic DNA to generate a truncated CKX polypeptide.

The nucleic acid constructs of the invention encoding a base editor comprising a sequence-specific DNA binding protein and an adenine deaminase polypeptide, and expression cassettes and/or vectors encoding the same may be used in combination with guide nucleic acids for modifying a target nucleic acid including, but not limited to, generation of A→G or T→C mutations in a target nucleic acid including, but not limited to, a plasmid sequence; generation of A→G or T→C mutations in a coding sequence to alter an amino acid identity; generation of A→G or T→C mutations in a coding sequence to generate a stop codon; generation of A→G or T→C mutations in a coding sequence to disrupt a start codon; generation of point mutations in genomic DNA to disrupt function; and/or generation of point mutations in genomic DNA to disrupt splice junctions.

The nucleic acid constructs of the invention comprising a CRISPR-Cas effector protein or a fusion protein thereof may be used in combination with a guide RNA (gRNA, CRISPR array, CRISPR RNA, crRNA), designed to function with the encoded CRISPR-Cas effector protein or domain, to modify a target nucleic acid. A guide nucleic acid useful with this invention comprises at least one spacer sequence and at least one repeat sequence. The guide nucleic acid is capable of forming a complex with the CRISPR-Cas nuclease domain encoded and expressed by a nucleic acid construct of the invention and the spacer sequence is capable of hybridizing to a target nucleic acid, thereby guiding the complex (e.g., a CRISPR-Cas effector fusion protein (e.g., CRISPR-Cas effector domain fused to a deaminase domain and/or a CRISPR-Cas effector domain fused to a peptide tag or an affinity polypeptide to recruit a deaminase domain and optionally, a UGI) to the target nucleic acid, wherein the target nucleic acid may be modified (e.g., cleaved or edited) or modulated (e.g., modulating transcription) by the deaminase domain.

As an example, a nucleic acid construct encoding a Cas9 domain linked to a cytosine deaminase domain (e.g., fusion protein) may be used in combination with a Cas9 guide nucleic acid to modify a target nucleic acid, wherein the cytosine deaminase domain of the fusion protein deaminates a cytosine base in the target nucleic acid, thereby editing the target nucleic acid. In a further example, a nucleic acid construct encoding a Cas9 domain linked to an adenine deaminase domain (e.g., fusion protein) may be used in combination with a Cas9 guide nucleic acid to modify a target nucleic acid, wherein the adenine deaminase domain of the fusion protein deaminates an adenosine base in the target nucleic acid, thereby editing the target nucleic acid.

Likewise, a nucleic acid construct encoding a Cas12a domain (or other selected CRISPR-Cas nuclease, e.g., C2c1, C2c3, Cas12b, Cas12c, Cas12d, Cas12e, Cas13a, Cas13b, Cas13c, Cas13d, Cas1, Cas1B, Cas2, Cas3, Cas3′, Cas3″, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4 (dinG), and/or Csf5) linked to a cytosine deaminase domain or adenine deaminase domain (e.g., fusion protein) may be used in combination with a Cas12a guide nucleic acid (or the guide nucleic acid for the other selected CRISPR-Cas nuclease) to modify a target nucleic acid, wherein the cytosine deaminase domain or adenine deaminase domain of the fusion protein deaminates a cytosine base in the target nucleic acid, thereby editing the target nucleic acid.

A “guide nucleic acid,” “guide RNA,” “gRNA,” “CRISPR RNA/DNA” “crRNA” or “crDNA” as used herein means a nucleic acid that comprises at least one spacer sequence, which is complementary to (and hybridizes to) a target DNA (e.g., protospacer), and at least one repeat sequence (e.g., a repeat of a Type V Cas12a CRISPR-Cas system, or a fragment or portion thereof; a repeat of a Type II Cas9 CRISPR-Cas system, or fragment thereof; a repeat of a Type V C2c1 CRISPR Cas system, or a fragment thereof; a repeat of a CRISPR-Cas system of, for example, C2c3, Cas12a (also referred to as Cpf1), Cas12b, Cas12c, Cas12d, Cas12e, Cas13a, Cas13b, Cas13c, Cas13d, Cas1, Cas1B, Cas2, Cas3, Cas3′, Cas3″, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4 (dinG), and/or Csf5, or a fragment thereof), wherein the repeat sequence may be linked to the 5′ end and/or the 3′ end of the spacer sequence. The design of a gRNA of this invention may be based on a Type I, Type II, Type III, Type IV, Type V, or Type VI CRISPR-Cas system.

In some embodiments, a Cas12a gRNA may comprise, from 5′ to 3′, a repeat sequence (full length or portion thereof (“handle”); e.g., pseudoknot-like structure) and a spacer sequence.

In some embodiments, a guide nucleic acid may comprise more than one repeat sequence-spacer sequence (e.g., 2, 3, 4, 5, 6, 7, 8, 9, 10, or more repeat-spacer sequences) (e.g., repeat-spacer-repeat, e.g., repeat-spacer-repeat-spacer-repeat-spacer-repeat-spacer-repeat-spacer, and the like). The guide nucleic acids of this invention are synthetic, human-made and not found in nature. A gRNA can be quite long and may be used as an aptamer (like in the MS2 recruitment strategy) or other RNA structures hanging off the spacer.

A “repeat sequence” as used herein, refers to, for example, any repeat sequence of a wild-type CRISPR Cas locus (e.g., a Cas9 locus, a Cas12a locus, a C2c1 locus, etc.) or a repeat sequence of a synthetic crRNA that is functional with the CRISPR-Cas effector protein encoded by the nucleic acid constructs of the invention. A repeat sequence useful with this invention can be any known or later identified repeat sequence of a CRISPR-Cas locus (e.g., Type I, Type II, Type III, Type IV, Type V or Type VI) or it can be a synthetic repeat designed to function in a Type I, II, III, IV, V or VI CRISPR-Cas system. A repeat sequence may comprise a hairpin structure and/or a stem loop structure. In some embodiments, a repeat sequence may form a pseudoknot-like structure at its 5′ end (i.e., “handle”). Thus, in some embodiments, a repeat sequence can be identical to or substantially identical to a repeat sequence from wild-type Type I CRISPR-Cas loci, Type II, CRISPR-Cas loci, Type III, CRISPR-Cas loci, Type IV CRISPR-Cas loci, Type V CRISPR-Cas loci and/or Type VI CRISPR-Cas loci. A repeat sequence from a wild-type CRISPR-Cas locus may be determined through established algorithms, such as using the CRISPRfinder offered through CRISPRdb (see, Grissa et al. Nucleic Acids Res. 35(Web Server issue):W52-7). In some embodiments, a repeat sequence or portion thereof is linked at its 3′ end to the 5′ end of a spacer sequence, thereby forming a repeat-spacer sequence (e.g., guide nucleic acid, guide RNA/DNA, crRNA, crDNA).

In some embodiments, a repeat sequence comprises, consists essentially of, or consists of at least 10 nucleotides depending on the particular repeat and whether the guide nucleic acid comprising the repeat is processed or unprocessed (e.g., about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 to 100 or more nucleotides, or any range or value therein). In some embodiments, a repeat sequence comprises, consists essentially of, or consists of about 10 to about 20, about 10 to about 30, about 10 to about 45, about 10 to about 50, about 15 to about 30, about 15 to about 40, about 15 to about 45, about 15 to about 50, about 20 to about 30, about 20 to about 40, about 20 to about 50, about 30 to about 40, about 40 to about 80, about 50 to about 100 or more nucleotides.

A repeat sequence linked to the 5′ end of a spacer sequence can comprise a portion of a repeat sequence (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more contiguous nucleotides of a wild type repeat sequence). In some embodiments, a portion of a repeat sequence linked to the 5′ end of a spacer sequence can be about five to about ten consecutive nucleotides in length (e.g., about 5, 6, 7, 8, 9, 10 nucleotides) and have at least 90% sequence identity (e.g., at least about 90%, 91%, 92%, 93%, 94%, 95%, 96%, 97%, 98%, 99%, or more) to the same region (e.g., 5′ end) of a wild type CRISPR Cas repeat nucleotide sequence. In some embodiments, a portion of a repeat sequence may comprise a pseudoknot-like structure at its 5′ end (e.g., “handle”).

A “spacer sequence” as used herein is a nucleotide sequence that is complementary to a target nucleic acid (e.g., target DNA) (e.g., protospacer) (e.g., a portion of consecutive nucleotides of a CKX gene, wherein the CKX gene (a) comprises a sequence having at least 80% sequence identity to any one of the nucleotide sequences of SEQ ID NOs:72, 73, 75, 76, 78, 79, 81, 82, 84, 87, 88, or 91; (b) comprises a region having at least 80% sequence identity to any one of the nucleotide sequences of SEQ ID NOs:93-98; and/or (c) encodes a polypeptide having at least 80% sequence identity to any one of the amino acid sequences of SEQ ID NOs:74, 77, 80, 83, 89, or 92. A spacer sequence can be fully complementary or substantially complementary (e.g., at least about 70% complementary (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to a target nucleic acid. In some embodiments, the spacer sequence can have one, two, three, four, or five mismatches as compared to the target nucleic acid, which mismatches can be contiguous or noncontiguous. In some embodiments, the spacer sequence can have 70% complementarity to a target nucleic acid. In other embodiments, the spacer nucleotide sequence can have 80% complementarity to a target nucleic acid. In still other embodiments, the spacer nucleotide sequence can have 85%, 90%, 95%, 96%, 97%, 98%, 99% or 99.5% complementarity, and the like, to the target nucleic acid (protospacer). In some embodiments, the spacer sequence is 100% complementary to the target nucleic acid. A spacer sequence may have a length from about 15 nucleotides to about 30 nucleotides (e.g., 15, 16, 17, 18, 19, 20, 21, 22, 23, 24, 25, 26, 27, 28, 29, or 30 nucleotides, or any range or value therein). Thus, in some embodiments, a spacer sequence may have complete complementarity or substantial complementarity over a region of a target nucleic acid (e.g., protospacer) that is at least about 15 nucleotides to about 30 nucleotides in length. In some embodiments, the spacer is about 20 nucleotides in length. In some embodiments, the spacer is about 21, 22, or 23 nucleotides in length.

In some embodiments, the 5′ region of a spacer sequence of a guide nucleic acid may be identical to a target DNA, while the 3′ region of the spacer may be substantially complementary to the target DNA (such as for example, a Type V CRISPR-Cas system), or the 3′ region of a spacer sequence of a guide nucleic acid may be identical to a target DNA, while the 5′ region of the spacer may be substantially complementary to the target DNA (such as for example, a Type II CRISPR-Cas system), and therefore, the overall complementarity of the spacer sequence to the target DNA may be less than 100%. Thus, for example, in a guide for a Type V CRISPR-Cas system, the first 1, 2, 3, 4, 5, 6, 7, 8, 9, 10 nucleotides in the 5′ region (i.e., seed region) of, for example, a 20 nucleotide spacer sequence may be 100% complementary to the target DNA, while the remaining nucleotides in the 3′ region of the spacer sequence are substantially complementary (e.g., at least about 70% complementary) to the target DNA. In some embodiments, the first 1 to 8 nucleotides (e.g., the first 1, 2, 3, 4, 5, 6, 7, 8, nucleotides, and any range therein) of the 5′ end of the spacer sequence may be 100% complementary to the target DNA, while the remaining nucleotides in the 3′ region of the spacer sequence are substantially complementary (e.g., at least about 50% complementary (e.g., 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to the target DNA.

As a further example, in a guide for a Type II CRISPR-Cas system, the first 1, 2, 3, 4, 5, 6, 7, 8, 9, 10 nucleotides in the 3′ region (i.e., seed region) of, for example, a 20 nucleotide spacer sequence may be 100% complementary to the target DNA, while the remaining nucleotides in the 5′ region of the spacer sequence are substantially complementary (e.g., at least about 70% complementary) to the target DNA. In some embodiments, the first 1 to 10 nucleotides (e.g., the first 1, 2, 3, 4, 5, 6, 7, 8, 9, 10 nucleotides, and any range therein) of the 3′ end of the spacer sequence may be 100% complementary to the target DNA, while the remaining nucleotides in the 5′ region of the spacer sequence are substantially complementary (e.g., at least about 50% complementary (e.g., at least about 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more or any range or value therein)) to the target DNA.

In some embodiments, a seed region of a spacer may be about 8 to about 10 nucleotides in length, about 5 to about 6 nucleotides in length, or about 6 nucleotides in length.

As used herein, a “target nucleic acid”, “target DNA,” “target nucleotide sequence,” “target region,” or a “target region in the genome” refers to a region of a plant's genome that is fully complementary (100% complementary) or substantially complementary (e.g., at least 70% complementary (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to a spacer sequence in a guide nucleic acid of this invention. A target region useful for a CRISPR-Cas system may be located immediately 3′ (e.g., Type V CRISPR-Cas system) or immediately 5′ (e.g., Type II CRISPR-Cas system) to a PAM sequence in the genome of the organism (e.g., a plant genome). A target region may be selected from any region of at least 15 consecutive nucleotides (e.g., 16, 17, 18, 19, 20, 21, 22, 23, 24, 25, 26, 27, 28, 29, 30 nucleotides, and the like) located immediately adjacent to a PAM sequence.

A “protospacer sequence” refers to the target double stranded DNA and specifically to the portion of the target DNA (e.g., or target region in the genome) that is fully or substantially complementary (and hybridizes) to the spacer sequence of the CRISPR repeat-spacer sequences (e.g., guide nucleic acids, CRISPR arrays, crRNAs).

In the case of Type V CRISPR-Cas (e.g., Cas12a) systems and Type II CRISPR-Cas (Cas9) systems, the protospacer sequence is flanked by (e.g., immediately adjacent to) a protospacer adjacent motif (PAM). For Type IV CRISPR-Cas systems, the PAM is located at the 5′ end on the non-target strand and at the 3′ end of the target strand (see below, as an example).

```
        5′-NNNNNNNNNNNNNNNNNNN-3′ RNA Spacer(SEQ ID NO: 42)
            |||||||||||||||||||
3′AAANNNNNNNNNNNNNNNNNNN-5′ Target strand (SEQ ID NO: 43)
    ||||
5′TTTNNNNNNNNNNNNNNNNNNN-3′ Non-target strand (SEQ ID NO: 44)
```

In the case of Type II CRISPR-Cas (e.g., Cas9) systems, the PAM is located immediately 3′ of the target region. The PAM for Type I CRISPR-Cas systems is located 5′ of the target strand. There is no known PAM for Type III CRISPR-Cas systems. Makarova et al. describes the nomenclature for all the classes, types and subtypes of CRISPR systems (Nature Reviews Microbiology 13:722-736 (2015)). Guide structures and PAMs are described in by R. Barrangou (Genome Biol. 16:247 (2015)).

Canonical Cas12a PAMs are T rich. In some embodiments, a canonical Cas12a PAM sequence may be 5′-TTN, 5′-TTTN, or 5′-TTTV. In some embodiments, canonical Cas9 (e.g., S. pyogenes) PAMs may be 5′-NGG-3′. In some embodiments, non-canonical PAMs may be used but may be less efficient.

Additional PAM sequences may be determined by those skilled in the art through established experimental and computational approaches. Thus, for example, experimental approaches include targeting a sequence flanked by all possible nucleotide sequences and identifying sequence members that do not undergo targeting, such as through the transformation of target plasmid DNA (Esvelt et al. 2013. Nat. Methods 10:1116-1121; Jiang et al. 2013. Nat. Biotechnol. 31:233-239). In some aspects, a computational approach can include performing BLAST searches of natural spacers to identify the original target DNA sequences in bacteriophages or plasmids and aligning these sequences to determine conserved sequences adjacent to the target sequence (Briner and Barrangou. 2014. Appl. Environ. Microbiol. 80:994-1001; Mojica et al. 2009. Microbiology 155:733-740).

In some embodiments, the present invention provides expression cassettes and/or vectors comprising the nucleic acid constructs of the invention (e.g., one or more components of an editing system of the invention). In some embodiments, expression cassettes and/or vectors comprising the nucleic acid constructs of the invention and/or one or more guide nucleic acids may be provided. In some embodiments, a nucleic acid construct of the invention encoding a base editor (e.g., a construct comprising a CRISPR-Cas effector protein and a deaminase domain (e.g., a fusion protein)) or the components for base editing (e.g., a CRISPR-Cas effector protein fused to a peptide tag or an affinity polypeptide, a deaminase domain fused to a peptide tag or an affinity polypeptide, and/or a UGI fused to a peptide tag or an affinity polypeptide), may be comprised on the same or on a separate expression cassette or vector from that comprising the one or more guide nucleic acids. When the nucleic acid construct encoding a base editor or the components for base editing is/are comprised on separate expression cassette(s) or vector(s) from that comprising the guide nucleic acid, a target nucleic acid may be contacted with (e.g., provided with) the expression cassette(s) or vector(s) encoding the base editor or components for base editing in any order from one another and the guide nucleic acid, e.g., prior to, concurrently with, or after the expression cassette comprising the guide nucleic acid is provided (e.g., contacted with the target nucleic acid).

Fusion proteins of the invention may comprise sequence-specific nucleic acid binding domains, CRISPR-Cas polypeptides, and/or deaminase domains fused to peptide tags or affinity polypeptides that interact with the peptide tags, as known in the art, for use in recruiting the deaminase to the target nucleic acid. Methods of recruiting may also comprise guide nucleic acids linked to RNA recruiting motifs and deaminases fused to affinity polypeptides capable of interacting with RNA recruiting motifs, thereby recruiting the deaminase to the target nucleic acid. Alternatively, chemical interactions may be used to recruit polypeptides (e.g., deaminases) to a target nucleic acid.

A peptide tag (e.g., epitope) useful with this invention may include, but is not limited to, a GCN4 peptide tag (e.g., Sun-Tag), a c-Myc affinity tag, an HA affinity tag, a His affinity tag, an S affinity tag, a methionine-His affinity tag, an RGD-His affinity tag, a FLAG octapeptide, a strep tag or strep tag II, a V5 tag, and/or a VSV-G epitope. In some embodiments, a peptide tag may also include phosphorylated tyrosines in specific sequence contexts recognized by SH2 domains, characteristic consensus sequences containing phosphoserines recognized by 14-3-3 proteins, proline rich peptide motifs recognized by SH3 domains, PDZ protein interaction domains or the PDZ signal sequences, and an AGO hook motif from plants. Peptide tags are disclosed in WO2018/136783 and U.S. Patent Application Publication No. 2017/0219596, which are incorporated by reference for their disclosures of peptide tags. Any epitope that may be linked to a polypeptide and for which there is a corresponding affinity polypeptide that may be linked to another polypeptide may be used with this invention as a peptide tag. A peptide tag may comprise or be present in one copy or in 2 or more copies of the peptide tag (e.g., multimerized peptide tag or multimerized epitope) (e.g., about 2, 3, 4, 5, 6, 7, 8, 9, 10, 11, 12, 13, 14, 15, 16, 17, 18, 9, 20, 21, 22, 23, 24, or 25 or more peptide tags). When multimerized, the peptide tags may be fused directly to one another or they may be linked to one another via one or more amino acids (e.g., 1, 2, 3, 4, 5, 6, 7, 8, 9, 10, 11, 12, 13, 14, 15, 16, 17, 18, 19, 20 or more amino acids, optionally about 3 to about 10, about 4 to about 10, about 5 to about 10, about 5 to about 15, or about 5 to about 20 amino acids, and the like, and any value or range therein. In some embodiments, an affinity polypeptide that interacts with/binds to a peptide tag may be an antibody. In some embodiments, the antibody may be a scFv antibody. In some embodiments, an affinity polypeptide that binds to a peptide tag may be synthetic (e.g., evolved for affinity interaction) including, but not limited to, an affibody, an anticalin, a monobody and/or a DARPin (see, e.g., Sha et al., Protein Sci. 26(5):910-924 (2017)); Gilbreth (Curr Opin Struc Biol 22(4):413-420 (2013)), U.S. Pat. No. 9,982,053, each of which are incorporated by reference in their entireties for the teachings relevant to affibodies, anticalins, monobodies and/or DARPins. Example peptide tag sequences and their affinity polypeptides include, but are not limited to, the amino acid sequences of SEQ ID NOs:45-47.

In some embodiments, a guide nucleic acid may be linked to an RNA recruiting motif, and a polypeptide to be recruited (e.g., a deaminase) may be fused to an affinity polypeptide that binds to the RNA recruiting motif, wherein the guide binds to the target nucleic acid and the RNA recruiting motif binds to the affinity polypeptide, thereby recruiting the polypeptide to the guide and contacting the target nucleic acid with the polypeptide (e.g., deaminase). In some embodiments, two or more polypeptides may be recruited to a guide nucleic acid, thereby contacting the target nucleic acid with two or more polypeptides (e.g., deaminases). Example RNA recruiting motifs and their affinity polypeptides include, but are not limited to, the sequences of SEQ ID NOs:48-58.

In some embodiments, a polypeptide fused to an affinity polypeptide may be a reverse transcriptase and the guide nucleic acid may be an extended guide nucleic acid linked to an RNA recruiting motif. In some embodiments, an RNA recruiting motif may be located on the 3′ end of the extended portion of an extended guide nucleic acid (e.g., 5′-3′, repeat-spacer-extended portion (RT template-primer binding site)-RNA recruiting motif). In some embodiments, an RNA recruiting motif may be embedded in the extended portion.

In some embodiments of the invention, an extended guide RNA and/or guide RNA may be linked to one or to two or more RNA recruiting motifs (e.g., 1, 2, 3, 4, 5, 6, 7, 8, 9, 10 or more motifs; e.g., at least 10 to about 25 motifs), optionally wherein the two or more RNA recruiting motifs may be the same RNA recruiting motif or different RNA recruiting motifs. In some embodiments, an RNA recruiting motif and corresponding affinity polypeptide may include, but is not limited, to a telomerase Ku binding motif (e.g., Ku binding hairpin) and the corresponding affinity polypeptide Ku (e.g., Ku heterodimer), a telomerase Sm7 binding motif and the corresponding affinity polypeptide Sm7, an MS2 phage operator stem-loop and the corresponding affinity polypeptide MS2 Coat Protein (MCP), a PP7 phage operator stem-loop and the corresponding affinity polypeptide PP7 Coat Protein (PCP), an SfMu phage Com stem-loop and the corresponding affinity polypeptide Com RNA binding protein, a PUF binding site (PBS) and the affinity polypeptide Pumilio/fem-3 mRNA binding factor (PUF), and/or a synthetic RNA-aptamer and the aptamer ligand as the corresponding affinity polypeptide. In some embodiments, the RNA recruiting motif and corresponding affinity polypeptide may be an MS2 phage operator stem-loop and the affinity polypeptide MS2 Coat Protein (MCP). In some embodiments, the RNA recruiting motif and corresponding affinity polypeptide may be a PUF binding site (PBS) and the affinity polypeptide Pumilio/fem-3 mRNA binding factor (PUF).

In some embodiments, the components for recruiting polypeptides and nucleic acids may those that function through chemical interactions that may include, but are not limited to, rapamycin-inducible dimerization of FRB—FKBP; Biotin-streptavidin; SNAP tag; Halo tag; CLIP tag; DmrA-DmrC heterodimer induced by a compound; bifunctional ligand (e.g., fusion of two protein-binding chemicals together, e.g., dihyrofolate reductase (DHFR).

In some embodiments, the nucleic acid constructs, expression cassettes or vectors of the invention that are optimized for expression in a plant may be about 70% to 100% identical (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100%) to the nucleic acid constructs, expression cassettes or vectors comprising the same polynucleotide(s) but which have not been codon optimized for expression in a plant.

Further provided herein are cells comprising one or more polynucleotides, guide nucleic acids, nucleic acid constructs, expression cassettes or vectors of the invention.

The nucleic acid constructs of the invention (e.g., a construct comprising a sequence specific nucleic acid binding domain, a CRISPR-Cas effector domain, a deaminase domain, reverse transcriptase (RT), RT template and/or a guide nucleic acid, etc.) and expression cassettes/vectors comprising the same may be used as an editing system of this invention for modifying target nucleic acids and/or their expression.

A target nucleic acid of any plant or plant part (or groupings of plants, for example, into a genus or higher order classification) may be modified (e.g., mutated, e.g., base edited, cleaved, nicked, etc.) using the polypeptides, polynucleotides, ribonucleoproteins (RNPs), nucleic acid constructs, expression cassettes, and/or vectors of the invention including an angiosperm, a gymnosperm, a monocot, a dicot, a C3, C4, CAM plant, a bryophyte, a fern and/or fern ally, a microalgae, and/or a macroalgae. A plant and/or plant part that may be modified as described herein may be a plant and/or plant part of any plant species/variety/cultivar. In some embodiments, a plant that may be modified as described herein is a monocot. In some embodiments, a plant that may be modified as described herein is a dicot.

The term “plant part,” as used herein, includes but is not limited to reproductive tissues (e.g., petals, sepals, stamens, pistils, receptacles, anthers, pollen, flowers, fruits, flower bud, ovules, seeds, embryos, nuts, kernels, ears, cobs and husks); vegetative tissues (e.g., petioles, stems, roots, root hairs, root tips, pith, coleoptiles, stalks, shoots, branches, bark, apical meristem, axillary bud, cotyledon, hypocotyls, and leaves); vascular tissues (e.g., phloem and xylem); specialized cells such as epidermal cells, parenchyma cells, chollenchyma cells, schlerenchyma cells, stomates, guard cells, cuticle, mesophyll cells; callus tissue; and cuttings. The term “plant part” also includes plant cells, including plant cells that are intact in plants and/or parts of plants, plant protoplasts, plant tissues, plant organs, plant cell tissue cultures, plant calli, plant clumps, and the like. As used herein, “shoot” refers to the above ground parts including the leaves and stems. As used herein, the term “tissue culture” encompasses cultures of tissue, cells, protoplasts and callus.

As used herein, “plant cell” refers to a structural and physiological unit of the plant, which typically comprise a cell wall but also includes protoplasts. A plant cell of the present invention can be in the form of an isolated single cell or can be a cultured cell or can be a part of a higher-organized unit such as, for example, a plant tissue (including callus) or a plant organ. In some embodiments, a plant cell can be an algal cell. A “protoplast” is an isolated plant cell without a cell wall or with only parts of the cell wall. Thus, in some embodiments of the invention, a transgenic cell comprising a nucleic acid molecule and/or nucleotide sequence of the invention is a cell of any plant or plant part including, but not limited to, a root cell, a leaf cell, a tissue culture cell, a seed cell, a flower cell, a fruit cell, a pollen cell, and the like. In some aspects of the invention, the plant part can be a plant germplasm. In some aspects, a plant cell can be non-propagating plant cell that does not regenerate into a plant.

“Plant cell culture” means cultures of plant units such as, for example, protoplasts, cell culture cells, cells in plant tissues, pollen, pollen tubes, ovules, embryo sacs, zygotes and embryos at various stages of development.

As used herein, a “plant organ” is a distinct and visibly structured and differentiated part of a plant such as a root, stem, leaf, flower bud, or embryo.

“Plant tissue” as used herein means a group of plant cells organized into a structural and functional unit. Any tissue of a plant in planta or in culture is included. This term includes, but is not limited to, whole plants, plant organs, plant seeds, tissue culture and any groups of plant cells organized into structural and/or functional units. The use of this term in conjunction with, or in the absence of, any specific type of plant tissue as listed above or otherwise embraced by this definition is not intended to be exclusive of any other type of plant tissue.

In some embodiments of the invention, a transgenic tissue culture or transgenic plant cell culture is provided, wherein the transgenic tissue or cell culture comprises a nucleic acid molecule/nucleotide sequence of the invention. In some embodiments, transgenes may be eliminated from a plant developed from the transgenic tissue or cell by breeding of the transgenic plant with a non-transgenic plant and selecting among the progeny for the plants comprising the desired gene edit and not the transgenes used in producing the edit.

Any plant comprising an endogenous CKX gene that is capable of regulating cytokinin balance in favor of active cytokinins in a plant may be modified as described herein to increase yield in the plant (e.g., increased seed number, increased seed size; increased pod number; or improved yield traits as a result of increased planting density of a plant of the invention versus planting a control plant at an increased density).

Non-limiting examples of plants that may be modified as described herein may include, but are not limited to, turf grasses (e.g., bluegrass, bentgrass, ryegrass, fescue), feather reed grass, tufted hair grass, miscanthus, arundo, switchgrass, vegetable crops, including artichokes, kohlrabi, arugula, leeks, asparagus, lettuce (e.g., head, leaf, romaine), malanga, melons (e.g., muskmelon, watermelon, crenshaw, honeydew, cantaloupe), cole crops (e.g., brussels sprouts, cabbage, cauliflower, broccoli, collards, kale, chinese cabbage, bok choy), cardoni, carrots, napa, okra, onions, celery, parsley, chick peas, parsnips, chicory, peppers, potatoes, cucurbits (e.g., marrow, cucumber, zucchini, squash, pumpkin, honeydew melon, watermelon, cantaloupe), radishes, dry bulb onions, rutabaga, eggplant, salsify, escarole, shallots, endive, garlic, spinach, green onions, squash, greens, beet (sugar beet and fodder beet), sweet potatoes, chard, horseradish, tomatoes, turnips, and spices; a fruit crop such as apples, apricots, cherries, nectarines, peaches, pears, plums, prunes, cherry, quince, fig, nuts (e.g., chestnuts, pecans, pistachios, hazelnuts, pistachios, peanuts, walnuts, macadamia nuts, almonds, and the like), citrus (e.g., clementine, kumquat, orange, grapefruit, tangerine, mandarin, lemon, lime, and the like), blueberries, black raspberries, boysenberries, cranberries, currants, gooseberries, loganberries, raspberries, strawberries, blackberries, grapes (wine and table), avocados, bananas, kiwi, persimmons, pomegranate, pineapple, tropical fruits, pomes, melon, mango, papaya, and lychee, a field crop plant such as clover, alfalfa, timothy, evening primrose, meadow foam, corn/maize (field, sweet, popcorn), hops, jojoba, buckwheat, safflower, quinoa, wheat, rice, barley, rye, millet, sorghum, oats, triticale, sorghum, tobacco, kapok, a leguminous plant (beans (e.g., green and dried), lentils, peas, soybeans), an oil plant (rape, canola, mustard, poppy, olive, sunflower, coconut, castor oil plant, cocoa bean, groundnut, oil palm), duckweed, Arabidopsis, a fiber plant (cotton, flax, hemp, jute), Cannabis (e.g., Cannabis sativa, Cannabis indica, and Cannabis ruderalis), lauraceae (cinnamon, camphor), or a plant such as coffee, sugar cane, tea, and natural rubber plants; and/or a bedding plant such as a flowering plant, a cactus, a succulent and/or an ornamental plant (e.g., roses, tulips, violets), as well as trees such as forest trees (broad-leaved trees and evergreens, such as conifers; e.g., elm, ash, oak, maple, fir, spruce, cedar, pine, birch, cypress, eucalyptus, willow), as well as shrubs and other nursery stock. In some embodiments, the nucleic acid constructs of the invention and/or expression cassettes and/or vectors encoding the same may be used to modify maize, soybean, wheat, canola, rice, tomato, pepper, or sunflower.

In some embodiments, a plant that may be modified as described herein may include, but is not limited to, corn, soybean, canola, wheat, rice, cotton, sugarcane, sugar beet, barley, oats, alfalfa, sunflower, safflower, oil palm, sesame, coconut, tobacco, potato, sweet potato, cassava, coffee, apple, plum, apricot, peach, cherry, pear, fig, banana, citrus, cocoa, avocado, olive, almond, walnut, strawberry, watermelon, pepper, grape, tomato, cucumber, or a Brassica spp (e.g., B. napus, B. oleraceae, B. rapa, B. juncea, and/or B. nigra). In some embodiments, a plant that may be modified as described herein is soybean (i.e., Glycine max).

Thus, plants or plant cultivars which are to be treated with preference in accordance with the invention include all plants which, through genetic modification, received genetic material which imparts particular advantageous useful properties (“traits”) to these plants. Examples of such properties are better plant growth, vigor, stress tolerance, standability, lodging resistance, nutrient uptake, plant nutrition, and/or yield, in particular improved growth, increased tolerance to high or low temperatures, increased tolerance to drought or to levels of water or soil salinity, enhanced flowering performance, easier harvesting, accelerated ripening, higher yields, higher quality and/or a higher nutritional value of the harvested products, better storage life and/or processability of the harvested products.

Further examples of such properties are an increased resistance against animal and microbial pests, such as against insects, arachnids, nematodes, mites, slugs and snails owing, for example, to toxins formed in the plants. Among DNA sequences encoding proteins which confer properties of tolerance to such animal and microbial pests, in particular insects, mention will particularly be made of the genetic material from Bacillus thuringiensis encoding the Bt proteins widely described in the literature and well known to those skilled in the art. Mention will also be made of proteins extracted from bacteria such as Photorhabdus (WO97/17432 and WO98/08932). In particular, mention will be made of the Bt Cry or VIP proteins which include the CrylA, CryIAb, CryIAc, CryIIA, CryIIIA, CryIIIB2, Cry9c Cry2Ab, Cry3Bb and CryIF proteins or toxic fragments thereof and also hybrids or combinations thereof, especially the CrylF protein or hybrids derived from a CrylF protein (e.g., hybrid CrylA-CrylF proteins or toxic fragments thereof), the CrylA-type proteins or toxic fragments thereof, preferably the CrylAc protein or hybrids derived from the CrylAc protein (e.g., hybrid CrylAb-CrylAc proteins) or the CrylAb or Bt2 protein or toxic fragments thereof, the Cry2Ae, Cry2Af or Cry2Ag proteins or toxic fragments thereof, the CrylA.105 protein or a toxic fragment thereof, the VIP3Aa19 protein, the VIP3Aa20 protein, the VIP3A proteins produced in the COT202 or COT203 cotton events, the VIP3Aa protein or a toxic fragment thereof as described in Estruch et al. (1996), Proc Natl Acad Sci US A. 28; 93(11):5389-94, the Cry proteins as described in WO2001/47952, the insecticidal proteins from Xenorhabdus (as described in WO98/50427), Serratia (particularly from S. entomophila) or Photorhabdus species strains, such as Tc-proteins from Photorhabdus as described in WO98/08932. Also any variants or mutants of any one of these proteins differing in some amino acids (1-10, preferably 1-5) from any of the above named sequences, particularly the sequence of their toxic fragment, or which are fused to a transit peptide, such as a plastid transit peptide, or another protein or peptide, is included herein.

Another and particularly emphasized example of such properties is conferred tolerance to one or more herbicides, for example imidazolinones, sulphonylureas, glyphosate or phosphinothricin. Among DNA sequences encoding proteins (i.e., polynucleotides of interest) which confer properties of tolerance to certain herbicides on the transformed plant cells and plants, mention will be particularly be made to the bar or PAT gene or the Streptomyces coelicolor gene described in WO2009/152359 which confers tolerance to glufosinate herbicides, a gene encoding a suitable EPSPS (5-Enolpyruvylshikimat-3-phosphat-Synthase) which confers tolerance to herbicides having EPSPS as a target, especially herbicides such as glyphosate and its salts, a gene encoding glyphosate-n-acetyltransferase, or a gene encoding glyphosate oxidoreductase. Further suitable herbicide tolerance traits include at least one ALS (acetolactate synthase) inhibitor (e.g., WO2007/024782), a mutated Arabidopsis ALS/AHAS gene (e.g., U.S. Pat. No. 6,855,533), genes encoding 2,4-D-monooxygenases conferring tolerance to 2,4-D (2,4-dichlorophenoxyacetic acid) and genes encoding Dicamba monooxygenases conferring tolerance to dicamba (3,6-dichloro-2-methoxybenzoic acid).

Further examples of such properties are increased resistance against phytopathogenic fungi, bacteria and/or viruses owing, for example, to systemic acquired resistance (SAR), systemin, phytoalexins, elicitors and also resistance genes and correspondingly expressed proteins and toxins.

Particularly useful transgenic events in transgenic plants or plant cultivars which can be treated with preference in accordance with the invention include Event 531/PV-GHBK04 (cotton, insect control, described in WO2002/040677), Event 1143-14A (cotton, insect control, not deposited, described in WO2006/128569); Event 1143-51B (cotton, insect control, not deposited, described in WO2006/128570); Event 1445 (cotton, herbicide tolerance, not deposited, described in US-A 2002-120964 or WO2002/034946); Event 17053 (rice, herbicide tolerance, deposited as PTA-9843, described in WO2010/117737); Event 17314 (rice, herbicide tolerance, deposited as PTA-9844, described in WO2010/117735); Event 281-24-236 (cotton, insect control—herbicide tolerance, deposited as PTA-6233, described in WO2005/103266 or US-A 2005-216969); Event 3006-210-23 (cotton, insect control—herbicide tolerance, deposited as PTA-6233, described in US-A 2007-143876 or WO2005/103266); Event 3272 (corn, quality trait, deposited as PTA-9972, described in WO2006/098952 or US-A 2006-230473); Event 33391 (wheat, herbicide tolerance, deposited as PTA-2347, described in WO2002/027004), Event 40416 (corn, insect control—herbicide tolerance, deposited as ATCC PTA-11508, described in WO 11/075593); Event 43A47 (corn, insect control—herbicide tolerance, deposited as ATCC PTA-11509, described in WO2011/075595); Event 5307 (corn, insect control, deposited as ATCC PTA-9561, described in WO2010/077816); Event ASR-368 (bent grass, herbicide tolerance, deposited as ATCC PTA-4816, described in US-A 2006-162007 or WO2004/053062); Event B16 (corn, herbicide tolerance, not deposited, described in US-A 2003-126634); Event BPS-CV127-9 (soybean, herbicide tolerance, deposited as NCIMB No. 41603, described in WO2010/080829); Event BLRI (oilseed rape, restoration of male sterility, deposited as NCIMB 41193, described in WO2005/074671), Event CE43-67B (cotton, insect control, deposited as DSM ACC2724, described in US-A 2009-217423 or WO2006/128573); Event CE44-69D (cotton, insect control, not deposited, described in US-A 2010-0024077); Event CE44-69D (cotton, insect control, not deposited, described in WO2006/128571); Event CE46-02A (cotton, insect control, not deposited, described in WO2006/128572); Event COT102 (cotton, insect control, not deposited, described in US-A 2006-130175 or WO2004/039986); Event COT202 (cotton, insect control, not deposited, described in US-A 2007-067868 or WO2005/054479); Event COT203 (cotton, insect control, not deposited, described in WO2005/054480)); Event DAS21606-3/1606 (soybean, herbicide tolerance, deposited as PTA-11028, described in WO2012/033794), Event DAS40278 (corn, herbicide tolerance, deposited as ATCC PTA-10244, described in WO2011/022469); Event DAS-44406-6/pDAB8264.44.06.1 (soybean, herbicide tolerance, deposited as PTA-11336, described in WO2012/075426), Event DAS-14536-7/pDAB8291.45.36.2 (soybean, herbicide tolerance, deposited as PTA-11335, described in WO2012/075429), Event DAS-59122-7 (corn, insect control—herbicide tolerance, deposited as ATCC PTA 11384, described in US-A 2006-070139); Event DAS-59132 (corn, insect control—herbicide tolerance, not deposited, described in WO2009/100188); Event DAS68416 (soybean, herbicide tolerance, deposited as ATCC PTA-10442, described in WO2011/066384 or WO2011/066360); Event DP-098140-6 (corn, herbicide tolerance, deposited as ATCC PTA-8296, described in US-A 2009-137395 or WO 08/112019); Event DP-305423-1 (soybean, quality trait, not deposited, described in US-A 2008-312082 or WO2008/054747); Event DP-32138-1 (corn, hybridization system, deposited as ATCC PTA-9158, described in US-A 2009-0210970 or WO2009/103049); Event DP-356043-5 (soybean, herbicide tolerance, deposited as ATCC PTA-8287, described in US-A 2010-0184079 or WO2008/002872); EventEE-I (brinjal, insect control, not deposited, described in WO 07/091277); Event Fil 17 (corn, herbicide tolerance, deposited as ATCC 209031, described in US-A 2006-059581 or WO 98/044140); Event FG72 (soybean, herbicide tolerance, deposited as PTA-11041, described in WO2011/063413), Event GA21 (corn, herbicide tolerance, deposited as ATCC 209033, described in US-A 2005-086719 or WO 98/044140); Event GG25 (corn, herbicide tolerance, deposited as ATCC 209032, described in US-A 2005-188434 or WO98/044140); Event GHB119 (cotton, insect control—herbicide tolerance, deposited as ATCC PTA-8398, described in WO2008/151780); Event GHB614 (cotton, herbicide tolerance, deposited as ATCC PTA-6878, described in US-A 2010-050282 or WO2007/017186); Event GJ11 (corn, herbicide tolerance, deposited as ATCC 209030, described in US-A 2005-188434 or WO98/044140); Event GM RZ13 (sugar beet, virus resistance, deposited as NCIMB-41601, described in WO2010/076212); Event H7-1 (sugar beet, herbicide tolerance, deposited as NCIMB 41158 or NCIMB 41159, described in US-A 2004-172669 or WO 2004/074492); Event JOPLIN1 (wheat, disease tolerance, not deposited, described in US-A 2008-064032); Event LL27 (soybean, herbicide tolerance, deposited as NCIMB41658, described in WO2006/108674 or US-A 2008-320616); Event LL55 (soybean, herbicide tolerance, deposited as NCIMB 41660, described in WO 2006/108675 or US-A 2008-196127); Event LLcotton25 (cotton, herbicide tolerance, deposited as ATCC PTA-3343, described in WO2003/013224 or US-A 2003-097687); Event LLRICE06 (rice, herbicide tolerance, deposited as ATCC 203353, described in U.S. Pat. No. 6,468,747 or WO2000/026345); Event LLRice62 (rice, herbicide tolerance, deposited as ATCC 203352, described in WO2000/026345), Event LLRICE601 (rice, herbicide tolerance, deposited as ATCC PTA-2600, described in US-A 2008-2289060 or WO2000/026356); Event LY038 (corn, quality trait, deposited as ATCC PTA-5623, described in US-A 2007-028322 or WO2005/061720); Event MIR162 (corn, insect control, deposited as PTA-8166, described in US-A 2009-300784 or WO2007/142840); Event MIR604 (corn, insect control, not deposited, described in US-A 2008-167456 or WO2005/103301); Event MON15985 (cotton, insect control, deposited as ATCC PTA-2516, described in US-A 2004-250317 or WO2002/100163); Event MON810 (corn, insect control, not deposited, described in US-A 2002-102582); Event MON863 (corn, insect control, deposited as ATCC PTA-2605, described in WO2004/011601 or US-A 2006-095986); Event MON87427 (corn, pollination control, deposited as ATCC PTA-7899, described in WO2011/062904); Event MON87460 (corn, stress tolerance, deposited as ATCC PTA-8910, described in WO2009/111263 or US-A 2011-0138504); Event MON87701 (soybean, insect control, deposited as ATCC PTA-8194, described in US-A 2009-130071 or WO2009/064652); Event MON87705 (soybean, quality trait—herbicide tolerance, deposited as ATCC PTA-9241, described in US-A 2010-0080887 or WO2010/037016); Event MON87708 (soybean, herbicide tolerance, deposited as ATCC PTA-9670, described in WO2011/034704); Event MON87712 (soybean, yield, deposited as PTA-10296, described in WO2012/051199), Event MON87754 (soybean, quality trait, deposited as ATCC PTA-9385, described in WO2010/024976); Event MON87769 (soybean, quality trait, deposited as ATCC PTA-8911, described in US-A 2011-0067141 or WO2009/102873); Event MON88017 (corn, insect control—herbicide tolerance, deposited as ATCC PTA-5582, described in US-A 2008-028482 or WO2005/059103); Event MON88913 (cotton, herbicide tolerance, deposited as ATCC PTA-4854, described in WO2004/072235 or US-A 2006-059590); Event MON88302 (oilseed rape, herbicide tolerance, deposited as PTA-10955, described in WO2011/153186), Event MON88701 (cotton, herbicide tolerance, deposited as PTA-11754, described in WO2012/134808), Event MON89034 (corn, insect control, deposited as ATCC PTA-7455, described in WO 07/140256 or US-A 2008-260932); Event MON89788 (soybean, herbicide tolerance, deposited as ATCC PTA-6708, described in US-A 2006-282915 or WO2006/130436); Event MSl 1 (oilseed rape, pollination control—herbicide tolerance, deposited as ATCC PTA-850 or PTA-2485, described in WO2001/031042); Event MS8 (oilseed rape, pollination control—herbicide tolerance, deposited as ATCC PTA-730, described in WO2001/041558 or US-A 2003-188347); Event NK603 (corn, herbicide tolerance, deposited as ATCC PTA-2478, described in US-A 2007-292854); Event PE-7 (rice, insect control, not deposited, described in WO2008/114282); Event RF3 (oilseed rape, pollination control—herbicide tolerance, deposited as ATCC PTA-730, described in WO2001/041558 or US-A 2003-188347); Event RT73 (oilseed rape, herbicide tolerance, not deposited, described in WO2002/036831 or US-A 2008-070260); Event SYHT0H2/SYN-000H2-5 (soybean, herbicide tolerance, deposited as PTA-11226, described in WO2012/082548), Event T227-1 (sugar beet, herbicide tolerance, not deposited, described in WO2002/44407 or US-A 2009-265817); Event T25 (corn, herbicide tolerance, not deposited, described in US-A 2001-029014 or WO2001/051654); Event T304-40 (cotton, insect control—herbicide tolerance, deposited as ATCC PTA-8171, described in US-A 2010-077501 or WO2008/122406); Event T342-142 (cotton, insect control, not deposited, described in WO2006/128568); Event TC1507 (corn, insect control—herbicide tolerance, not deposited, described in US-A 2005-039226 or WO2004/099447); Event VIP1034 (corn, insect control—herbicide tolerance, deposited as ATCC PTA-3925, described in WO2003/052073), Event 32316 (corn, insect control-herbicide tolerance, deposited as PTA-11507, described in WO2011/084632), Event 4114 (corn, insect control-herbicide tolerance, deposited as PTA-11506, described in WO2011/084621), event EE-GM3/FG72 (soybean, herbicide tolerance, ATCC Accession No PTA-11041) optionally stacked with event EE-GM1/LL27 or event EE-GM2/LL55 (WO2011/063413A2), event DAS-68416-4 (soybean, herbicide tolerance, ATCC Accession No PTA-10442, WO2011/066360A1), event DAS-68416-4 (soybean, herbicide tolerance, ATCC Accession No PTA-10442, WO2011/066384A1), event DP-040416-8 (corn, insect control, ATCC Accession No PTA-11508, WO2011/075593A1), event DP-043A47-3 (corn, insect control, ATCC Accession No PTA-11509, WO2011/075595A1), event DP-004114-3 (corn, insect control, ATCC Accession No PTA-11506, WO2011/084621A1), event DP-032316-8 (corn, insect control, ATCC Accession No PTA-11507, WO2011/084632A1), event MON-88302-9 (oilseed rape, herbicide tolerance, ATCC Accession No PTA-10955, WO2011/153186A1), event DAS-21606-3 (soybean, herbicide tolerance, ATCC Accession No. PTA-11028, WO2012/033794A2), event MON-87712-4 (soybean, quality trait, ATCC Accession No. PTA-10296, WO2012/051199A2), event DAS-44406-6 (soybean, stacked herbicide tolerance, ATCC Accession No. PTA-11336, WO2012/075426A1), event DAS-14536-7 (soybean, stacked herbicide tolerance, ATCC Accession No. PTA-11335, WO2012/075429A1), event SYN-000H2-5 (soybean, herbicide tolerance, ATCC Accession No. PTA-11226, WO2012/082548A2), event DP-061061-7 (oilseed rape, herbicide tolerance, no deposit No available, WO2012071039A1), event DP-073496-4 (oilseed rape, herbicide tolerance, no deposit No available, US2012131692), event 8264.44.06.1 (soybean, stacked herbicide tolerance, Accession No PTA-11336, WO2012075426A2), event 8291.45.36.2 (soybean, stacked herbicide tolerance, Accession No. PTA-11335, WO2012075429A2), event SYHT0H2 (soybean, ATCC Accession No. PTA-11226, WO2012/082548A2), event MON88701 (cotton, ATCC Accession No PTA-11754, WO2012/134808A1), event KK179-2 (alfalfa, ATCC Accession No PTA-11833, WO2013/003558A1), event pDAB8264.42.32.1 (soybean, stacked herbicide tolerance, ATCC Accession No PTA-11993, WO2013/010094A1), event MZDT09Y (corn, ATCC Accession No PTA-13025, WO2013/012775A1).

The genes/events (e.g., polynucleotides of interest), which impart the desired traits in question, may also be present in combinations with one another in the transgenic plants. Examples of transgenic plants which may be mentioned are the important crop plants, such as cereals (wheat, rice, triticale, barley, rye, oats), maize, soya beans, potatoes, sugar beet, sugar cane, tomatoes, peas and other types of vegetable, cotton, tobacco, oilseed rape and also fruit plants (with the fruits apples, pears, citrus fruits and grapes), with particular emphasis being given to maize, soya beans, wheat, rice, potatoes, cotton, sugar cane, tobacco and oilseed rape. Traits which are particularly emphasized are the increased resistance of the plants to insects, arachnids, nematodes and slugs and snails, as well as the increased resistance of the plants to one or more herbicides.

Commercially available examples of such plants, plant parts or plant seeds that may be treated with preference in accordance with the invention include commercial products, such as plant seeds, sold or distributed under the GENUITY®, DROUGHTGARD®, SMARTSTAX®, RIB COMPLETE®, ROUNDUP READY®, VT DOUBLE PRO®, VT TRIPLE PRO®, BOLLGARD II®, ROUNDUP READY 2 YIELD®, YIELDGARD®, ROUNDUP READY® 2 XTEN^{DTm}, INTACTA RR2 PRO®, VISTIVE GOLD®, and/or XTENDFLEX™ trade names.

The invention will now be described with reference to the following examples. It should be appreciated that these examples are not intended to limit the scope of the claims to the invention but rather are intended to be exemplary of certain embodiments. Any variations in the exemplified methods that occur to the skilled artisan are intended to fall within the scope of the invention.

### EXAMPLES

### Example 1. Gene-Editing and Selection of Edited Plants

Disarmed Agrobacterium tumefaciens was used to introduce a T-DNA cassette expressing a selectable marker and CRISPR Cas gene editing components targeted to create double-strand breaks in CKX gene coding sequences and thereby generate CKX knock-outs. The T-DNA further expressed crRNAs comprising spacers selected from SEQ ID Nos. 99-113 (Table 1). These spacers are programmed to target the CKX coding genes. Different combinations of spacers were used to generate particular combinations of desired CKX knock-outs, including CKX1/2/3, CKX1/2/3/4 and CKX1/3/5/6, as shown in Table 2.

PCR and next generation sequencing (NGS) were used to confirm that intended genetic changes were achieved. Genomic DNA was isolated from leaf tissue and used as a template in PCR reactions using primers specific to the CKX genes targeted. The amplified products were subsequently sequenced and characterized to confirm the genetic changes. SEQ ID NOs:-114-284 provide examples of mutations achieved using the editing systems as described herein. Table 3 provides each of the example edits along with plant identification number (CEID), the edited locus, which corresponds to an edited CKX gene (Table 4), the start position of the deletion relevant to the wild-type genomic sequence and the deletion length.

First-generation edited events (E0) of interest were selfed and progeny (E1 generation) were selected from the segregating population. E1 plants comprising out-of-frame deletions in the coding region of the desired CKX genes were planted and grown and E2 seed was harvested. E2 seed was planted for phenotypic testing.

TABLE 1
Spacer sequences and targeted CKX genes in Soybeans

| CKX | Public gene ID | SEQ ID NO: | Spacer Name | Spacer seq |
|---|---|---|---|---|
| CKX1 | Glyma.15G170300 | PWg120386 | SEQ ID NO: | TCCTCCTGTTCATAACCATAACA |
| | | | 99 | |
| | | PWg120389 | SEQ ID NO: | TGACGACCCAGAGGCCCTCCAGG |
| | | | 100 | |
| | | PWg120425 | SEQ ID NO: | ACATACTTCATCCTCCTGTTCAT |
| | | | 101 | |
| CKX2 | Glyma.09G063900 | PWg120385 | SEQ ID NO: | TCCTCCTGCTCATAACCATAACA |
| | | | 102 | |
| | | PWg120388 | SEQ ID NO: | CAACGACCCAGAGGCCCTCCAGG |
| | | | 103 | |
| | | PWg120433 | SEQ ID NO: | GGAGGCGGCGTTAGGGCCTTCAT |
| | | | 104 | |
| CKX3 | Glyma.17G054500 | PWg120384 | SEQ ID NO: | TACTACTGCTAGTAACCATAACC |
| | | | 105 | |
| | | PWg120387 | SEQ ID NO: | TGATGACCCTGAAACCATTCAAA |
| | | | 106 | |
| | | PWg120432 | SEQ ID NO: | ACAGTGAATATCAAACGGGTTAT |
| | | | 107 | |
| CKX4 | Glyma.04g028900 | PWg120426 | SEQ ID NO: | TGGCTGTCAACAACAACAAGCTT |
| | | | 108 | |
| | | PWg120427 | SEQ ID NO: | CCGAAGCCGCCTCCAGTTCCAAC |
| | | | 109 | |
| CKX5 | Glyma.09g225400 | PWg120428 | SEQ ID NO: | TCAAAAGCTTCGCCTTCACGATA |
| | | | 110 | |
| | | PWg120429 | SEQ ID NO: | CTGAAATCAATTCCCCTTGAAGG |
| | | | 111 | |
| CKX6 | Glyma.09g063500 | PWg120430 | SEQ ID NO: | ACAACTATGGTTGTGCTATTGCT |
| | | | 112 | |
| | | PWg120431 | SEQ ID NO: | ACATGCCTCAACCGATTATGGGC |
| | | | 113 | |

TABLE 2
Constructs and Spacers used to target CKX genes in Soybean

| | | | Spacer SEQ ID | |
|---|---|---|---|---|
| Construct: | CKX | Public gene ID | No. | Spacer sequence |
| pWISE1092 | CKX1 | Glyma.15G170300 | SEQ ID NO: 99 | TCCTCCTGTTCATAACCATAACA |
| | | | SEQ ID NO: 100 | TGACGACCCAGAGGCCCTCCAGG |
| | CKX2 | Glyma.09G063900 | SEQ ID NO: 102 | TCCTCCTGCTCATAACCATAACA |
| | | | SEQ ID NO: 103 | CAACGACCCAGAGGCCCTCCAGG |
| | CKX3 | Glyma.17G054500 | SEQ ID NO: 105 | TACTACTGCTAGTAACCATAACC |
| | | | SEQ ID NO: 106 | TGATGACCCTGAAACCATTCAAA |
| pWISE1335 | CKX1 | Glyma.15G170300 | SEQ ID NO: 100 | TGACGACCCAGAGGCCCTCCAGG |
| | | | SEQ ID NO: 101 | ACATACTTCATCCTCCTGTTCA |
| | CKX3 | Glyma.17G054500 | SEQ ID NO: 106 | TGATGACCCTGAAACCATTCAAA |
| | | | SEQ ID NO: 107 | ACAGTGAATATCAAACGGGTTAT |
| | CKX5 | Glyma.09g225400 | SEQ ID NO: 110 | TCAAAAGCTTCGCCTTCACGATA |
| | | | SEQ ID NO: 111 | CTGAAATCAATTCCCCTTGAAGG |
| | CKX6 | Glyma.09g063500 | SEQ ID NO: 112 | ACAACTATGGTTGTGCTATTGCT |
| | | | SEQ ID NO: 113 | ACATGCCTCAACCGATTATGGGC |
| pWISE1336 | CKX1 | Glyma.15G170300 | SEQ ID NO: 99 | TCCTCCTGTTCATAACCATAACA |
| | | | SEQ ID NO: 100 | TGACGACCCAGAGGCCCTCCAGG |
| | CKX2 | Glyma.09G063900 | SEQ ID NO: 103 | CAACGACCCAGAGGCCCTCCAGG |
| | | | SEQ ID NO: 104 | GGAGGCGGCGTTAGGGCCTTCAT |
| | CKX3 | Glyma.17G054500 | SEQ ID NO: 105 | TACTACTGCTAGTAACCATAACC |
| | | | SEQ ID NO: 106 | TGATGACCCTGAAACCATTCAAA |
| | CKX4 | Glyma.04g028900 | SEQ ID NO: 109 | CCGAAGCCGCCTCCAGTTCCAAC |
| | | | SEQ ID NO: 108 | TGGCTGTCAACAACAACAAGCTT |

TABLE 3
Obtained Edits in CKX genes in Soybean

| Plant ID - LOCUS - Start Position: Deletion Size (D) | Construct | SEQ ID NO: |
|---|---|---|
| CE20753-LOCUS116-803: 26D | pWISE1092 | SEQ ID NO: 114 |
| CE20753-LOCUS115-2038: 2D | pWISE1092 | SEQ ID NO: 115 |
| CE20600-LOCUS116-708: 5D | pWISE1092 | SEQ ID NO: 116 |
| CE20600-LOCUS116-654: 56D, 815: 11D | pWISE1092 | SEQ ID NO: 117 |
| CE20600-LOCUS113-797: 142D | pWISE1092 | SEQ ID NO: 118 |
| CE48659-LOCUS116-710: 13D | pWISE1335 | SEQ ID NO: 119 |
| CE48659-LOCUS116-710: 8D | pWISE1335 | SEQ ID NO: 120 |
| CE48659-LOCUS115-1894: 9D, 2038: 4D | pWISE1335 | SEQ ID NO: 121 |
| CE48659-LOCUS114-785: 4D | pWISE1335 | SEQ ID NO: 122 |
| CE48637-LOCUS116-711: 5D | pWISE1335 | SEQ ID NO: 123 |
| CE48637-LOCUS116-703: 14D | pWISE1335 | SEQ ID NO: 124 |
| CE48637-LOCUS115-1898: 12D | pWISE1335 | SEQ ID NO: 125 |
| CE48637-LOCUS115-1897: 6D, 2036: 15D | pWISE1335 | SEQ ID NO: 126 |
| CE48637-LOCUS114-770: 21D | pWISE1335 | SEQ ID NO: 127 |
| CE48637-LOCUS114-691: 16D | pWISE1335 | SEQ ID NO: 128 |
| CE48637-LOCUS107-1566: 11D | pWISE1335 | SEQ ID NO: 129 |
| CE48618-LOCUS116-713: 3D | pWISE1335 | SEQ ID NO: 130 |
| CE48618-LOCUS116-707: 7D | pWISE1335 | SEQ ID NO: 131 |
| CE48618-LOCUS115-1899: 9D | pWISE1335 | SEQ ID NO: 132 |
| CE48618-LOCUS115-1897: 11D | pWISE1335 | SEQ ID NO: 133 |
| CE48618-LOCUS114-783: 8D | pWISE1335 | SEQ ID NO: 134 |
| CE48618-LOCUS114-700: 8D, 781: 9D | pWISE1335 | SEQ ID NO: 135 |
| CE48618-LOCUS107-1578: 131D | pWISE1335 | SEQ ID NO: 136 |
| CE48618-LOCUS107-1573: 15D | pWISE1335 | SEQ ID NO: 137 |
| CE48596-LOCUS116-705: 15D | pWISE1335 | SEQ ID NO: 138 |
| CE48596-LOCUS115-2038: 4D | pWISE1335 | SEQ ID NO: 139 |
| CE48596-LOCUS114-699: 10D | pWISE1335 | SEQ ID NO: 140 |
| CE48596-LOCUS114-699: 9D | pWISE1335 | SEQ ID NO: 141 |
| CE48596-LOCUS107-1698: 11D | pWISE1335 | SEQ ID NO: 142 |
| CE48280-LOCUS115-1897: 5D | pWISE1335 | SEQ ID NO: 143 |
| CE48280-LOCUS115-1889: 29D | pWISE1335 | SEQ ID NO: 144 |
| CE48280-LOCUS114-775: 77D | pWISE1335 | SEQ ID NO: 145 |
| CE48280-LOCUS107-1608: 202D | pWISE1335 | SEQ ID NO: 146 |
| CE48280-LOCUS107-1575: 6D | pWISE1335 | SEQ ID NO: 147 |
| CE48131-LOCUS116-806: 20D | pWISE1335 | SEQ ID NO: 148 |
| CE48131-LOCUS114-775: 15D | pWISE1335 | SEQ ID NO: 149 |
| CE48131-LOCUS114-698: 10D | pWISE1335 | SEQ ID NO: 150 |
| CE48116-LOCUS116-712: 6D | pWISE1335 | SEQ ID NO: 151 |
| CE48116-LOCUS116-703: 13D | pWISE1335 | SEQ ID NO: 152 |
| CE48116-LOCUS115-1899: 5D, 2038: 5D | pWISE1335 | SEQ ID NO: 153 |
| CE48116-LOCUS115-1897: 6D, 2038: 7D | pWISE1335 | SEQ ID NO: 154 |
| CE48116-LOCUS114-700: 11D, 764: 26D | pWISE1335 | SEQ ID NO: 155 |
| CE48116-LOCUS114-694: 14D, 772: 15D | pWISE1335 | SEQ ID NO: 156 |
| CE48116-LOCUS107-1576: 135D | pWISE1335 | SEQ ID NO: 157 |
| CE48116-LOCUS107-1553: 28D, 1697: 12D | pWISE1335 | SEQ ID NO: 158 |
| CE48108-LOCUS116-710: 6D | pWISE1335 | SEQ ID NO: 159 |
| CE48108-LOCUS116-702: 15D, 801: 28D | pWISE1335 | SEQ ID NO: 160 |
| CE48108-LOCUS115-1899: 9D | pWISE1335 | SEQ ID NO: 161 |
| CE48108-LOCUS114-699: 8D, 763: 60D | pWISE1335 | SEQ ID NO: 162 |
| CE48108-LOCUS107-1578: 3D, 1664: 65D | pWISE1335 | SEQ ID NO: 163 |
| CE48108-LOCUS107-1576: 14D, 1697: 15D | pWISE1335 | SEQ ID NO: 164 |
| CE48101-LOCUS116-713: 1D | pWISE1335 | SEQ ID NO: 165 |
| CE48101-LOCUS115-1897: 9D | pWISE1335 | SEQ ID NO: 166 |
| CE48101-LOCUS115-1825: 84D | pWISE1335 | SEQ ID NO: 167 |
| CE48101-LOCUS107-1607: 101D | pWISE1335 | SEQ ID NO: 168 |
| CE48036-LOCUS115-1899: 7D | pWISE1335 | SEQ ID NO: 169 |
| CE48036-LOCUS114-699: 13D | pWISE1335 | SEQ ID NO: 170 |
| CE31767-LOCUS116-709: 7D | pWISE1335 | SEQ ID NO: 171 |
| CE31767-LOCUS115-1900: 8D, 2024: 31D | pWISE1335 | SEQ ID NO: 172 |
| CE31767-LOCUS107-1578: 4D | pWISE1335 | SEQ ID NO: 173 |
| CE31743-LOCUS116-712: 7D | pWISE1335 | SEQ ID NO: 174 |
| CE31743-LOCUS116-712: 6D | pWISE1335 | SEQ ID NO: 175 |
| CE31743-LOCUS115-1899: 5D | pWISE1335 | SEQ ID NO: 176 |
| CE31743-LOCUS115-1887: 13D | pWISE1335 | SEQ ID NO: 177 |
| CE31743-LOCUS107-1577: 10D | pWISE1335 | SEQ ID NO: 178 |
| CE31693-LOCUS116-715: 1D | pWISE1335 | SEQ ID NO: 179 |
| CE31693-LOCUS116-707: 32D | pWISE1335 | SEQ ID NO: 180 |
| CE31693-LOCUS115-1899: 9D | pWISE1335 | SEQ ID NO: 181 |
| CE31693-LOCUS115-1899: 5D | pWISE1335 | SEQ ID NO: 182 |
| CE31693-LOCUS114-698: 29D | pWISE1335 | SEQ ID NO: 183 |
| CE31693-LOCUS107-1701: 3D | pWISE1335 | SEQ ID NO: 184 |
| CE31693-LOCUS107-1579: 130D | pWISE1335 | SEQ ID NO: 185 |
| CE31664-LOCUS116-711: 8D | pWISE1335 | SEQ ID NO: 186 |
| CE31664-LOCUS116-707: 16D | pWISE1335 | SEQ ID NO: 187 |
| CE31664-LOCUS115-1897: 6D, 2036: 10D | pWISE1335 | SEQ ID NO: 188 |
| CE31664-LOCUS114-700: 11D | pWISE1335 | SEQ ID NO: 189 |
| CE31638-LOCUS116-710: 12D | pWISE1335 | SEQ ID NO: 190 |
| CE31638-LOCUS116-708: 13D | pWISE1335 | SEQ ID NO: 191 |
| CE31638-LOCUS115-1899: 7D, 2037: 8D | pWISE1335 | SEQ ID NO: 192 |
| CE31638-LOCUS115-1892: 23D | pWISE1335 | SEQ ID NO: 193 |
| CE31638-LOCUS114-780: 4D | pWISE1335 | SEQ ID NO: 194 |
| CE31638-LOCUS107-1701: 10D | pWISE1335 | SEQ ID NO: 195 |
| CE31638-LOCUS107-1573: 15D | pWISE1335 | SEQ ID NO: 196 |
| CE31577-LOCUS115-1898: 12D | pWISE1335 | SEQ ID NO: 197 |
| CE31577-LOCUS114-698: 10D | pWISE1335 | SEQ ID NO: 198 |
| CE31532-LOCUS116-710: 75D, 822: 6D | pWISE1335 | SEQ ID NO: 199 |
| CE31532-LOCUS116-706: 16D | pWISE1335 | SEQ ID NO: 200 |
| CE31532-LOCUS115-1900: 9D | pWISE1335 | SEQ ID NO: 201 |
| CE31532-LOCUS107-1700: 6D | pWISE1335 | SEQ ID NO: 202 |
| CE31532-LOCUS107-1563: 11D | pWISE1335 | SEQ ID NO: 203 |
| CE31492-LOCUS116-712: 66D | pWISE1335 | SEQ ID NO: 204 |
| CE31492-LOCUS116-710: 11D | pWISE1335 | SEQ ID NO: 205 |
| CE31492-LOCUS115-1897: 11D | pWISE1335 | SEQ ID NO: 206 |
| CE31492-LOCUS107-1576: 134D | pWISE1335 | SEQ ID NO: 207 |
| CE31492-LOCUS107-1556: 153D | pWISE1335 | SEQ ID NO: 208 |
| CE28077-LOCUS115-1902: 3D | pWISE1335 | SEQ ID NO: 209 |
| CE28077-LOCUS115-1898: 11D | pWISE1335 | SEQ ID NO: 210 |
| CE28077-LOCUS107-1693: 16D | pWISE1335 | SEQ ID NO: 211 |
| CE28024-LOCUS107-1573: 11D | pWISE1335 | SEQ ID NO: 212 |
| CE27997-LOCUS115-1901: 5D | pWISE1335 | SEQ ID NO: 213 |
| CE27956-LOCUS115-1898: 11D | pWISE1335 | SEQ ID NO: 214 |
| CE27956-LOCUS115-1898: 7D | pWISE1335 | SEQ ID NO: 215 |
| CE27956-LOCUS114-700: 3D | pWISE1335 | SEQ ID NO: 216 |
| CE27956-LOCUS107-1686: 23D | pWISE1335 | SEQ ID NO: 217 |
| CE27929-LOCUS116-712: 8D | pWISE1335 | SEQ ID NO: 218 |
| CE27929-LOCUS116-708: 11D | pWISE1335 | SEQ ID NO: 219 |
| CE27929-LOCUS115-1898: 13D | pWISE1335 | SEQ ID NO: 220 |
| CE49952-LOCUS113-867: 1D | pWISE1336 | SEQ ID NO: 221 |
| CE49927-LOCUS116-703: 125D | pWISE1336 | SEQ ID NO: 222 |
| CE49927-LOCUS116-702: 19D, 819: 4D | pWISE1336 | SEQ ID NO: 223 |
| CE49927-LOCUS115-2045: 1D | pWISE1336 | SEQ ID NO: 224 |
| CE49927-LOCUS113-862: 8D | pWISE1336 | SEQ ID NO: 225 |
| CE49927-LOCUS113-859: 13D | pWISE1336 | SEQ ID NO: 226 |
| CE49927-LOCUS105-1554: 7D | pWISE1336 | SEQ ID NO: 227 |
| CE49900-LOCUS116-704: 6D | pWISE1336 | SEQ ID NO: 228 |
| CE49900-LOCUS113-860: 25D | pWISE1336 | SEQ ID NO: 229 |
| CE49900-LOCUS105-1539: 22D | pWISE1336 | SEQ ID NO: 230 |
| CE49820-LOCUS116-707: 7D | pWISE1336 | SEQ ID NO: 231 |
| CE49820-LOCUS116-704: 6D | pWISE1336 | SEQ ID NO: 232 |
| CE49820-LOCUS116-576: 3D, 596: 116D | pWISE1336 | SEQ ID NO: 233 |
| CE49820-LOCUS113-853: 18D | pWISE1336 | SEQ ID NO: 234 |
| CE49820-LOCUS105-1556: 5D | pWISE1336 | SEQ ID NO: 235 |
| CE49819-LOCUS115-1908: 5D, 2038: 4D | pWISE1336 | SEQ ID NO: 236 |
| CE49819-LOCUS115-1904: 6D, 2038: 4D | pWISE1336 | SEQ ID NO: 237 |
| CE49819-LOCUS113-859: 13D | pWISE1336 | SEQ ID NO: 238 |
| CE49819-LOCUS113-838: 39D | pWISE1336 | SEQ ID NO: 239 |
| CE49819-LOCUS105-1555: 5D | pWISE1336 | SEQ ID NO: 240 |
| CE49819-LOCUS105-1553: 8D | pWISE1336 | SEQ ID NO: 241 |
| CE49804-LOCUS116-703: 17D, 818: 10D | pWISE1336 | SEQ ID NO: 242 |
| CE49804-LOCUS116-703: 17D | pWISE1336 | SEQ ID NO: 243 |
| CE49804-LOCUS113-862: 9D | pWISE1336 | SEQ ID NO: 244 |
| CE49804-LOCUS113-854: 16D | pWISE1336 | SEQ ID NO: 245 |
| CE49804-LOCUS105-1554: 6D | pWISE1336 | SEQ ID NO: 246 |
| CE49804-LOCUS105-1550: 47D | pWISE1336 | SEQ ID NO: 247 |
| CE49799-LOCUS105-1553: 10D | pWISE1336 | SEQ ID NO: 248 |
| CE49760-LOCUS116-704: 18D, 819: 16D | pWISE1336 | SEQ ID NO: 249 |
| CE49760-LOCUS113-865: 9D | pWISE1336 | SEQ ID NO: 250 |
| CE49760-LOCUS105-1525: 39D | pWISE1336 | SEQ ID NO: 251 |
| CE29267-LOCUS116-816: 8D | pWISE1336 | SEQ ID NO: 252 |
| CE29267-LOCUS116-703: 14D | pWISE1336 | SEQ ID NO: 253 |
| CE29267-LOCUS105-1553: 9D | pWISE1336 | SEQ ID NO: 254 |
| CE29267-LOCUS105-1552: 10D | pWISE1336 | SEQ ID NO: 255 |
| CE29257-LOCUS116-706: 8D | pWISE1336 | SEQ ID NO: 256 |
| CE29257-LOCUS116-705: 122D | pWISE1336 | SEQ ID NO: 257 |
| CE29257-LOCUS113-860: 7D | pWISE1336 | SEQ ID NO: 258 |
| CE29257-LOCUS105-1660: 16D | pWISE1336 | SEQ ID NO: 259 |
| CE29257-LOCUS105-1552: 7D | pWISE1336 | SEQ ID NO: 260 |
| CE29233-LOCUS116-708: 5D | pWISE1336 | SEQ ID NO: 261 |
| CE29233-LOCUS116-702: 19D | pWISE1336 | SEQ ID NO: 262 |
| CE29233-LOCUS115-1904: 14D | pWISE1336 | SEQ ID NO: 263 |
| CE29233-LOCUS113-863: 9D | pWISE1336 | SEQ ID NO: 264 |
| CE29233-LOCUS113-859: 13D | pWISE1336 | SEQ ID NO: 265 |
| CE29233-LOCUS105-1553: 47D | pWISE1336 | SEQ ID NO: 266 |
| CE29233-LOCUS105-1546: 6D | pWISE1336 | SEQ ID NO: 267 |
| CE27443-LOCUS116-704: 10D | pWISE1336 | SEQ ID NO: 268 |
| CE27443-LOCUS113-857: 16D | pWISE1336 | SEQ ID NO: 269 |
| CE27326-LOCUS116-707: 8D | pWISE1336 | SEQ ID NO: 270 |
| CE27248-LOCUS116-704: 6D, 820: 5D | pWISE1336 | SEQ ID NO: 271 |
| CE27248-LOCUS116-703: 12D | pWISE1336 | SEQ ID NO: 272 |
| CE27248-LOCUS115-2038: 4D | pWISE1336 | SEQ ID NO: 273 |
| CE27248-LOCUS113-861: 10D | pWISE1336 | SEQ ID NO: 274 |
| CE27248-LOCUS105-1552: 9D | pWISE1336 | SEQ ID NO: 275 |
| CE27247-LOCUS116-702: 14D | pWISE1336 | SEQ ID NO: 276 |
| CE27247-LOCUS116-701: 21D | pWISE1336 | SEQ ID NO: 277 |
| CE27247-LOCUS113-860: 11D | pWISE1336 | SEQ ID NO: 278 |
| CE27247-LOCUS113-848: 17D | pWISE1336 | SEQ ID NO: 279 |
| CE27220-LOCUS116-705: 7D | pWISE1336 | SEQ ID NO: 280 |
| CE27220-LOCUS116-704: 6D | pWISE1336 | SEQ ID NO: 281 |
| CE27220-LOCUS105-1550: 13D | pWISE1336 | SEQ ID NO: 282 |
| CE27218-LOCUS116-704: 6D | pWISE1336 | SEQ ID NO: 283 |
| CE27218-LOCUS116-702: 12D | pWISE1336 | SEQ ID NO: 284 |

TABLE 4
LOCUS - CKX correlation

| LOCUS | CKX Gene | SEQ ID NO: (genomic) |
|---|---|---|
| 115 | 1 | 72 |
| 113 | 2 | 75 |
| 116 | 3 | 78 |
| 105 | 4 | 81 |
| 114 | 5 | 84 |
| 107 | 6 | 87 |

### Example 2. Phenotype Evaluation E0 Plant CE20600, CKX2 and CKX3 Knock-Out

The E0 plant CE20600 was selected for further phenotype evaluation. The E0 plant was self-pollinated to generate the E1 population and a single plant from the E1 population was allowed to self-pollinate to generate the E2 population. The CE20600 plant described in Example 1 has edits in the CKX genes (SEQ ID NO:116-118), which are expected to knock out the genes CKX2 (SEQ ID NO:75) and CKX3 (SEQ ID NO:78).

Phenotyping data was collected included count measurements per plant for node count (plant, mainstem and branches), branch count, seed count, plant height, and dry seed weight. From these averages, calculations for seed per pod, nodes per branch, pods per node (plant, mainstem, and branches), and 100 seed weight were generated. A statistically significant difference (p=0.06) at a 10% level was observed in the average number of seeds per pod when comparing CE20600 (E2 generation) with a transformation control such that the CE20600 plant contained a greater number of seeds per pod than the transformation control.

A highly statistically significant difference (p<0.05) in plant height was observed between CE20600 (E2 generation) and the transformation control such that the CE20600 plant was taller than the transformation control. The number of pods on the mainstem and the number of nodes on the mainstem were also statistically different between CE20600 and the transformation control, with the CE20600 plant showing an increase in the number of pods and in the number of nodes on the mainstem, the latter being expected due to the difference in plant height.

There was no strong statistical evidence of a difference in the number of seeds per plant, number of branches, number of nodes or the number of pods between CE20600 and the transformation control.

A highly statistically significant (p<0.05) difference in 100 seed weight between CE20600 (E2 generation) and the transformation control with the 100 seed weight of CE20600 being less than the 100 seed weight of the transformation control.

Taken together, this data suggests that the combination of knock-out of CKX2 and CKX3 may lead to a yield increase when CE20600 is grown in a field environment.

### Example 3: Greenhouse Phenotype Analysis of CE48101 and CE48659

The E0 plants CE48101 and CE48659 were selected for further phenotype evaluation and the E0 plant was self-pollinated to generate the E1 population. The CE48101 plant described in Example 1 has edits in CKX genes (SEQ ID NO:165-168), which are expected to knock out the genes CKX1 (SEQ ID NO:72), CKX3 (SEQ ID NO:78) and CKX6 (SEQ ID NO:87). The CE48659 plant described in Example 1 has edits in the CKX genes (SEQ ID NO:120-122) which are expected to knock out the genes CKX1 (SEQ ID NO:72), CKX3 (SEQ ID NO:78) and CKX5 (SEQ ID NO:84). The edited alleles of the CKX genes in both CE48101 and CE48659 were found to be segregating in the E1 generation such that various combinations of edits were identified.

The E1 generation was evaluated at 110 days after sowing for a variety of phenotypes and compared to transformation control plants and the data is summarized below in Table 5. In general, the CE48101, CE48659 and control plants have a lot of vegetative biomass (more than usual) and very few set pods.

TABLE 5

| | | | | | | | | Pods per | Pods |
|---|---|---|---|---|---|---|---|---|---|
| | | No. of | Stem | Nodes on | No. of | Pod on | Pods on | node on | per |
| Family | Genotype | plants | thickness | Mainstem | branches | branches | Mainstem | mainstem | plant |
| CE48101 | CKX1, | 7 | 19.01 | 21.86 | 15.14 | 15.00 | 12.43 | 0.56 | 27.43 |
| | CKX3, | | | | | | | | |
| | CKX6 | | | | | | | | |
| | CKX1, | 2 | 20.54 | 22.50 | 17.50 | 17.00 | 7.50 | 0.33 | 24.50 |
| | CKX6 | | | | | | | | |
| CE48659 | CKX1, | 4 | 18.77 | 19.75 | 14.25 | 9.50 | 9.25 | 0.46 | 18.75 |
| | CKX3 | | | | | | | | |
| | CKX1, | 6 | 19.78 | 19.33 | 15.17 | 11.83 | 5.83 | 0.30 | 17.67 |
| | CKX3, | | | | | | | | |
| | CKX5 | | | | | | | | |
| | CKX3, | 4 | 19.92 | 18.75 | 14.00 | 15.25 | 7.00 | 0.36 | 22.25 |
| | CKX5 | | | | | | | | |
| WT | WT | 8 | 19.09 | 21.63 | 14.88 | 17.25 | 13.13 | 0.59 | 30.38 |

A two-tailed T-test did not detect any significant differences between CE48101 and CE48659 when either is compared to the wild type control, except for the decrease in the number of nodes on the mainstem in the CE48659 (CKX1, 3; CKX1, 3, 5; and CKX3, 5) family.

### Example 4: Greenhouse Phenotype Analysis of CE28077, Knock-Out CKX1 and CKX6

The E0 plant CE28077 was selected for further phenotype evaluation and the E0 plant was self-pollinated to generate the E1 population. The CE28077 plant described in Example 1 has edits in the CKX genes (SEQ ID NO:209-211) which are expected to knock out the genes CKX1 (SEQ ID NO:72), and CKX6 (SEQ ID NO:87).

No statistically significant (p value=0.38) difference in the average number of seeds per pod was found between the transformation control and the CE28077. Also, no statistically significant increase was observed for any of the yield-based traits, including count measurements per plant for node count (plant, mainstem and branches), branch count, seed count, plant height, and dry seed weight. From these averages, calculations for seed per pod, nodes per branch, pods per node (plant, mainstem, and branches), and 100 seed weight between the transformation control and CE28077. However, there was an observational increase in CE28077 for the number of nodes on the plant, as well as the number of branches on the plant. These observations suggest that the knock-out allele combination of CKX1 and CKX6 may give rise to architectural changes that could increase overall yield which can be evaluated in a field planting setting rather than a greenhouse environment.

### Example 5: Greenhouse Phenotype Analysis of CE29267, CE27443, and CE29257

Phenotyping data was collected on the E2 population of CE29267, CE27443 and CE29257. The E2 population was produced by allowing the E0 generation to self-pollinate giving rise to the E1 population. A single plant from the E1 population was allowed to self-pollinate to give rise to the E2 population which was evaluated for phenotypes in the greenhouse. The CE29267 plant contains edited CKX genes (SEQ ID NO:253-255) such that the genes CKX3 (SEQ ID NO:78) and CKX4 (SEQ ID NO:81) are expected to be knocked out. The CE27443 plant contains edited CKX genes (SEQ ID NO:268-269) such that the genes CKX2 (SEQ ID NO:75) and CKX3 (SEQ ID NO:78) are expected to be knocked out. The CE29257 plant contains edited CKX genes (SEQ ID NO:256-260) such that the genes CKX2 (SEQ ID NO:75), CKX3 (SEQ ID NO:78) and CKX4 (SEQ ID NO:81) are expected to be knocked out.

Phenotypes were evaluated by comparing CE29267, CE27443 and CE29257 against a transformation control line. Count measurements were collected per plant for number of mainstem nodes, number of pods (plant, mainstem and branches), and number of seeds per plant. In addition, a quantitative measurement for plant height was taken. From this data, two additional phenotypic traits were calculated for analysis, the number of seed per pod and number of pods per node for the mainstem.

There was no statistically significant support (i.e., p<0.05) for a difference in overall plant yield or the average seeds per pod when comparing CE29267, CE27443 or CE29257 against a wildtype, non-edited, control.

There was a statistically significant (p-value=0.061) increase in the number of pods per node on the mainstem and total pods on the mainstem for CE29267 as compared to the transformation control; however, this increase did not transfer to the overall number of pods per plant. With respect to CE29267, there was strong statistically significant decrease in the pod count on branches as compared to the transformation control. As for the pods on the mainstem, this decrease could be attributed to the presence of tall plants in the CE29267 population. The reduction in the pods on branches for the CE29267 translated to an overall lower pod count for this line as compared to the transformation control.

No statistically significant increase was noted for the total pod count for any of the lines CE29267, CE27443 or CE29257 when compared to the transformation control. In addition, there was no statistically significant difference in plant height between for any of the lines CE29267, CE27443 or CE29257 and the transformation control; however, there was observational evidence in CE29267 (knockout of CKX3 and CKX4) may give rise to architectural changes in the plant habit that may lead to an increase in yield when the plants are grown in a field environment.

### Example 6: Greenhouse Phenotype Evaluation of Knock-Out Edits in CKX1, CKX3 and CKX6

Phenotyping data was collected on the E2 population of CE31532 and CE31492. The E2 population was produced by allowing the E0 plant to self-pollinate giving rise to the E1 population. A single plant from the E1 population was allowed to self-pollinate to give rise to the E2 population which was evaluated for phenotypes in the greenhouse. The CE31532 plant contains edited CKX genes SEQ ID NO:199-203, and the CE31492 plant contains edited CKX genes SEQ ID NO:204-208. The edited CKX genes in both CE31532 and CE31492 are expected to give rise to knock-outs of all three of CKX1 (SEQ ID NO:72), CKX3 (SEQ ID NO:78) and CKX6 (SEQ ID NO:87).

Phenotypes were evaluated by comparing CE31532 and CE31492 against a transformation control line. Count measurements were collected per plant for number of mainstem nodes, number of pods (plant, mainstem and branches), and number of seeds per plant. In addition, a quantitative measurement for plant height was taken. From this data, two additional phenotypic traits were calculated for analysis, the number of seed per pod and number of pods per node for the mainstem.

There was no statistically significant (p<0.05) difference in the average number of seeds per pod when comparing CE31532 or CE31492 to a wild type, non-edited, control. There was strong statistical support of a lower overall seeds per plant in CE31532 and CE31492 as compared to the wild type, non-edited control; however, this lower seed count was balanced by a statistically lower pod count as well.

The pod counts for the branches and the mainstem indicated a statistically significant (p-value >0.001) decrease in the number of pods on the branches in the CE31532 and CE31492 lines as compared to the control. This reduction in pods was not observed when comparing the number of pods on the mainstem. There was no statistically significant increase in the average pods per node on the mainstem.

A statistically significant difference was observed as an increase in the number of nodes on the mainstem for CE31532 (p-value=0.01) in relation to the unedited control. In parallel, it was observed that CE31532 had a statistically significant increase in height (p-value=0) which would account for the increase in the number of nodes.

The architectural changes observed suggest that the knock-out combination of CKX1, CKX3 and CKX6 may result in yield changes which can be measured in a field environment.

### Example 7: Greenhouse Phenotype Evaluation of Knock-Outs in CKX1, CKX3, CKX5 and CKX6

The plants CE48618, CE48108 and CE48637 were selected for further phenotype analysis. All three of these plants contain edited versions of all 4 genes CKX1 (SEQ ID NO:72), CKX3 (SEQ ID NO:78), CKX5 (SEQ ID NO:84) and CKX6 (SEQ ID NO:87) with these genes being expected to be knock-outs in these plants. The E0 plants CE48618, CE48108 and CE48637 were self-pollinated to generate the E1 population. As described in Example 1, the CE48618 plant has edited sequences SEQ ID NO:130-137, CE48108 has edited sequences SEQ ID NO:159-164 and CE48637 has edited sequences SEQ ID NO:123-129.

The E1 populations were grown in pots in the greenhouse and evaluated at the R6 stage, which is the stage of growth where the seeds have formed but are not yet drying down. The R6 stage is the green bean stage where the total pod weight peaks and seed growth is in a rapid phase. The leaves on the lowest nodes of the plant begin to yellow. This stage is approximately 110 days after sowing of the seed.

The numbers of plants per genotype were too low for statistical analysis; however, it was observed that there was a trend for a reduced number of branches in the CE48108 E1 plants, and an increase in the number of pods on mainstem as compared to the wildtype, non-edited control plants. No difference in the number of seeds per plant or in the number of seeds per pod was observed; however, it should be noted that the sample size was very small.

The architectural changes observed in CE48108 suggest that the knock-out of CKX1, CKX3, CKX5 and CKX6 may lead to an increase in yield when grown in a field environment.

### Example 8: Greenhouse Phenotype Analysis of CE20753

Phenotyping data was collected on the E2 population of CE20753. The E2 population was produced by allowing the E0 generation to self-pollinate giving rise to the E1 population. A single plant from the E1 population was allowed to self-pollinate to give rise to the E2 population which was evaluated for phenotypes in the greenhouse. As described in Example 1, CE20753 contains edited CKX genes (SEQ ID NO:114-115), which are expected to give rise to knock-outs of CKX1 (SEQ ID NO:72) and CKX3 (SEQ ID NO:78).

Phenotypes were evaluated by comparing CE20753 against a transformation control line. Count measurements were collected per plant for number of mainstem nodes, number of pods (plant, mainstem and branches), and number of seeds per plant. In addition, a quantitative measurement for plant height was taken. From this data, two additional phenotypic traits were calculated for analysis, the number of seed per pod and number of pods per node for the mainstem.

There was strong statistical support of lower values in CE20753 as compared to the transformation control line for the phenotype of average number of seeds per pod and the number of seeds per plant. With respect to pod count traits, there was no statistically significant difference in the total number of pods per plant, average number of pods per node on the mainstem, or number pods on the mainstem for CE20753 as compared to the transformation control. No statistically significant difference was observed for the count of pods on branches or for plant height between CE20753 edited progeny and the transformation control.

### Example 9: Greenhouse Phenotype Analysis CE29233

Phenotyping data was collected on the E2 population of CE29233. The E2 population was produced by allowing the E0 generation to self-pollinate giving rise to the E1 population. A single plant from the E1 population was allowed to self-pollinate to give rise to the E2 population which was evaluated for phenotypes in the greenhouse. As described in Example 1, CE29233 contains edited CKX genes (SEQ ID NO:261-267) which are expected to give rise to knock-outs of CKX1 (SEQ ID NO:72), CKX3 (SEQ ID NO:78), CKX2 (SEQ ID NO:75) and CKX4 (SEQ ID NO:81). The edited alleles of the CKX genes in CE29233 were found to be segregating in the E1 generation such that various combinations of edits were identified in the E2 population evaluated.

Phenotypes were evaluated by comparing CE29233 against a transformation control line. Count measurements were collected per plant for number of mainstem nodes, number of pods (plant, mainstem and branches), and number of seeds per plant. In addition, a quantitative measurement for plant height was taken. From this data, two additional phenotypic traits were calculated for analysis, the number of seed per pod and number of pods per node for the mainstem.

A statistically significant increase was observed in the average number of pods per node on the mainstem, number of pods on the mainstem and number of nodes on the mainstem of the CE29233 plants with edits in the CXK2 and CKX4 genes when compared to the transformation control line. This was determined by comparing with the transformation control and the difference for these phenotypes was significant to the 0.05 level (p-values <=0.01).

There was limited evidence of a reduction in the number of pods on branches for CE29233 plants with edits in CXK2 and CKX4 (p-value=0.094).

In the CE29233 plants with edits in the CKX1, CKX2 and CKX4 genes, there was evidence for an increase in number of nodes on the mainstem (p-value 0.00). However, there was strong evidence that these plants exhibited a decrease in the average number of seeds per pod, number of seed per plant and number of pods on branches (p-values=0.00).

We did find evidence that the CE29233 plants with edits in CXK2 and CKX4 deletion showed an increase in the number of pods and nodes on the mainstem with limited evidence of a decrease in the number of pods on branches. However, there was no associated significant difference in the number of seeds on the plant.

We also found evidence that the CE29233 plants with edits in the CKX1, CKX2 and CKX4 genes had an increase in the number of nodes on the mainstem along with a decrease in the number of pods on branches.

### Example 10: Greenhouse Phenotype Analysis CE31638

Phenotyping data was collected on the E2 population of CE31638. The E2 population was produced by allowing the E0 generation to self-pollinate giving rise to the E1 population. A single plant from the E1 population was allowed to self-pollinate to give rise to the E2 population which was evaluated for phenotypes in the greenhouse. As described in Example 1, CE31638 contains edited CKX genes (SEQ ID NO:190-196) which are expected to give rise to knock-outs of CKX1 (SEQ ID NO:72), CKX3 (SEQ ID NO:78), CKX5 (SEQ ID NO:84) and CKX6 (SEQ ID NO:87). The edited alleles of the CKX genes in CE31638 segregated in the E1 generation and various combinations of these edited alleles were identified in the E2 population.

Phenotypes were evaluated by comparing CE31638 against a transformation control line. Count measurements were collected per plant for number of mainstem nodes, number of pods (plant, mainstem and branches), and number of seeds per plant. In addition, a quantitative measurement for plant height was taken. From this data, two additional phenotypic traits were calculated for analysis, the number of seed per pod and number of pods per node for the mainstem.

When comparing the various CKX knock-out allele combinations in CE31638 with the wild type, non-edited control, there was no statistically significant evidence of an increase in any of the traits at the p<0.05 level. There is very limited evidence for an increase in the average number of pods per node on the mainstem for the CKX1, CKX3, CKX5 and CKX6 knock-out combination (p-value 0.156). There was evidence of a reduction in the number of pods on branches and the number of pods per plant for the knock-out combination of CKX1, CKX5 and CKX6 (p-value=0.019 and 0.021 respectively).

There is observational evidence that the knock-out combination of CKX1, CKX3, CKX5 and CKX6 may alter the average number of pods per node on the mainstem and there may also be an upward trend for the number of seeds per plant when compared to the wild type, non-edited, control. In addition, both of the genotypes (e.g., knock-out of CKX1, CKX3, CKX5, CKX6, and knock-out of CKX1, CKX5 and CKX6) trended above the wild type, non-edited, control for the average number of pods per node on the mainstem and number of pods on the mainstem. Taken together, this data suggests that both of the CKX1, CKX3, CKX5 and CKX6; and CKX1, CKX5 and CKX6 knock-outs could increase plant yield when the plants are grown in a field environment.

### Example 11. Greenhouse Nursery Phenotype Evaluation

Selected E2 seed families were placed into greenhouse nurseries to increase seeds of a set of cytokinin oxidase gene editing events. To take advantage of nursery grow-out, phenotypic observations of these plants were made on a single plant basis. The E2 seed families that were evaluated are listed below in Table 5.

Within each family, the phenotypic changes observed to be most consistent among different plants of the event included plant height change, enhancement of pod setting, reduction of internode length, changes on branching number and position and delayed leaf senescence. In addition, there were a few additional phenotypic changes observed when comparing the individual E2 families, including the number of leaflet(s) and the number of seeds per pod.

Overall, these phenotypic observations were made at the single plant level. More than half of the E2 seed families produced similar or a higher number of seeds than that of the control plants. However, due to some of nursery practices such as no randomization of the nursery planting design, topping some plants that were too high and variation of growth conditions, the seed number data listed in Table 5 is just for monitoring off-types, not for quantification or comparison. The E2 families CE43708, CE55653 and CE59145 did show an average of a 5% increase in seed number when compared to non-edited/control plants in the same environment.

TABLE 5
E2 seed families

| | | Average | |
|---|---|---|---|
| Construct # | E2 Family name | seed#/plant | Plant # |
| Control | | 781 | 6 |
| PWISE1092 | CE43708 | 956 | 6 |
| PWISE1336 | CE55549 | 587 | 6 |
| PWISE1336 | CE55634 | 572 | 5 |
| PWISE1336 | CE55653 | 820 | 6 |
| PWISE1336 | CE55653 | 555 | 5 |
| PWISE1336 | CE55704 | 559 | 6 |
| PWISE1336 | CE55799 | 645 | 5 |
| PWISE1336 | CE59145 | 896 | 6 |
| PWISE1336 | CE64036 | 800 | 5 |
| PWISE1335 | CE48974 | 496 | 6 |
| PWISE1335 | CE49021 | 783 | 5 |
| PWISE1335 | CE73381 | 732 | 6 |
| PWISE1335 | CE76573 | 764 | 5 |
| PWISE1335 | CE76674 | 791 | 5 |
| PWISE1335 | CE76755 | 724 | 6 |

The foregoing is illustrative of the present invention and is not to be construed as limiting thereof. The invention is defined by the following claims, with equivalents of the claims to be included therein.

1. A plant or plant part thereof comprising at least one non-natural mutation in at least one endogenous Cytokinin Oxidase/Dehydrogenase (CKX) gene encoding a CKX protein.

2-6. (canceled)

7. The plant or part thereof of claim 1, wherein the at least one non-natural mutation results in a truncated CKX protein.

8. The plant or part thereof of claim 1, wherein the at least one non-natural mutation results in a 3′ end truncation of the CKX gene, which produces a truncated CKX protein or no protein.

9. (canceled)

10. The plant or part thereof of claim 1, wherein the endogenous CKX gene is an endogenous CKX1 gene, which encodes a CKX1 protein, an endogenous CKX2 gene, which encodes a CKX2 protein, an endogenous CKX3 gene, which encodes a CKX3 protein, an endogenous CKX4 gene, which encodes a CKX4 protein, an endogenous CKX5 gene, which encodes a CKX5 protein, or an endogenous CKX6 gene, which encodes a CKX6 protein, or any combination thereof.

11. The plant or part thereof of claim 10, wherein the at least one non-natural mutation is a mutation in at least two of the endogenous CKX genes of CKX1, CKX2, CKX3, CKX4, CKX5 and/or CKX6 gene, in any combination.

12. The plant or part thereof of claim 10, wherein the at least one non-natural mutation is a mutation in at least three of the endogenous CKX genes of CKX1, CKX2, CKX3, CKX4, CKX5 or CKX6 gene, in any combination.

13. (canceled)

14. The plant or part thereof of claim 1,
wherein the endogenous CKX gene is
a CKX1 gene that (a) comprises a sequence having at least 80% sequence identity to the nucleotide sequence of SEQ ID NO:72 or SEQ ID NO:73; (b) comprises a region having at least 80% sequence identity to any one of the nucleotide sequences of SEQ ID NO:93; and/or (c) encodes a polypeptide comprising a sequence having at least 80% sequence identity to the amino acid sequence of SEQ ID NO:74;
a CKX2 gene that (a) comprises a sequence having at least 80% sequence identity to the nucleotide sequence of SEQ ID NO:75 or SEQ ID NO:76; (b) comprises a region having at least 80% sequence identity to the nucleotide sequence of SEQ ID NO:94; and/or (c) encodes a polypeptide comprising a sequence having at least 80% sequence identity to the amino acid sequence of SEQ ID NO:77;
a CKX3 gene that (a) comprises a sequence having at least 80% sequence identity to the nucleotide sequence of SEQ ID NO:78 or SEQ ID NO:79; (b) comprises a region having at least 80% sequence identity to the nucleotide sequence of SEQ ID NO:95; and/or (c) encodes a polypeptide comprising a sequence having at least 80% sequence identity to the amino acid sequence of SEQ ID NO:80;
a CKX4 gene that (a) comprises a sequence having at least 80% sequence identity to the nucleotide sequence of SEQ ID NO:81 or SEQ ID NO:82; (b) comprises a region having at least 80% sequence identity to the nucleotide sequence of SEQ ID NO:96; and/or (c) encodes a polypeptide comprising a sequence having at least 80% sequence identity to the amino acid sequence of SEQ ID NO:83;
a CKX5 gene that (a) comprises a sequence having at least 80% sequence identity to the nucleotide sequence of SEQ ID NO:84 or SEQ ID NO:91; (b) comprises a region having at least 80% sequence identity to the nucleotide sequence of SEQ ID NO:97; and/or (c) encodes a polypeptide comprising a sequence having at least 80% sequence identity to the amino acid sequence of SEQ ID NO:92; and/or
a CKX6 gene that (a) comprises a sequence having at least 80% sequence identity to the nucleotide sequence of SEQ ID NO:87 or SEQ ID NO:88; (b) comprises a region having at least 80% sequence identity to the nucleotide sequence of SEQ ID NO:98; and/or (c) encodes a polypeptide comprising a sequence having at least 80% sequence identity to the amino acid sequence of SEQ ID NO:89.

15-18. (canceled)

19. The plant or part thereof of claim 1, wherein the plant is soybean.

20. A plant cell, comprising an editing system comprising:
(a) a CRISPR-associated effector protein; and
(b) a guide nucleic acid (gRNA, gDNA, crRNA, crDNA, sgRNA, sgDNA) comprising a spacer sequence with complementarity to an endogenous target gene encoding a CKX protein in the plant cell.

21-23. (canceled)

24. The plant cell of claim 1, wherein the endogenous target gene comprises a sequence having at least 80% sequence identity to any one of the nucleotide sequences of SEQ ID NOs: 72, 73, 75, 76, 78, 79, 81, 82, 84, 87, 88, or 91, and/or comprises a region having at least 80% sequence identity to any one of the nucleotide sequences of SEQ ID NOs:93-98.

25. The plant cell of claim 1, wherein the CKX protein comprises at least 80% sequence identity to any one of the amino acid sequences SEQ ID NOs:74, 77, 80, 83, 89, or 92.

26. The plant cell of claim 1, wherein the guide nucleic acid comprises any one of the nucleotide sequences of SEQ ID NOs:99-113.

27. A plant regenerated from the plant part of claim 1.

28. The plant of claim 27, wherein the plant comprises one or more mutated CKX genes comprising a nucleotide sequence having at least 90% sequence identity to any one of SEQ ID NOs:114-284, or an combination thereof.

29. (canceled)

30. A plant cell comprising at least one non-natural mutation within an endogenous Cytokinin Oxidase/Dehydrogenase (CKX) gene that results in a null allele or knockout of the CKX gene, wherein the at least one non-natural mutation is a base substitution, base insertion or a base deletion that is introduced using an editing system that comprises a nucleic acid binding domain that binds to a target site in the CKX gene.

31. The plant cell of claim 30, wherein the endogenous CKX gene is a CKX1 gene, a CKX2 gene, a CKX3 gene, a CKX4 gene, a CKX5 gene, and/or a CKX6 gene, or any combination thereof.

32-33. (canceled)

34. The plant cell of claim 30, wherein the endogenous CKX gene comprises a sequence having at least 80% sequence identity to any one of the nucleotide sequences of SEQ ID NOs:72, 73, 75, 76, 78, 79, 81, 82, 84, 87, 88, or 91, comprises a region having at least 80% sequence identity to any one of the nucleotide sequences of SEQ ID NOs:93-98 and/or encodes a polypeptide having at least 80% identity to any one of the amino acid sequences of SEQ ID NOs: 74, 77, 80, 83, 89, or 92.

35. The plant cell of claim 30, wherein the target site is within a region of the CKX gene, the region comprising a sequence having at least 80% sequence identity to a sequence comprising:
(a) about nucleotide 1884 to about nucleotide 2060 of the nucleotide sequence of SEQ ID NO:72 (CKX1) or about nucleotide 28 to about nucleotide 204 of the nucleotide sequence of SEQ ID NO:73 (CKX1) (e.g., SEQ ID NO:93);
(b) about nucleotide 803 to about nucleotide 955 of the nucleotide sequence of SEQ ID NO:75 (CKX2) or about nucleotide 38 to about nucleotide 190 of the nucleotide sequence of SEQ ID NO:76 (CKX2) (e.g., SEQ ID NO:94);
(c) about nucleotide 692 to about nucleotide 826 of the nucleotide sequence of SEQ ID NO:78 (CKX3) or about nucleotide 35 to about nucleotide 169 of the nucleotide sequence of SEQ ID NO:79 (CKX3) (e.g., SEQ ID NO:95);
(d) about nucleotide 1540 to about nucleotide 1689 of the nucleotide sequence of SEQ ID NO:81 (CKX4) or about nucleotide 2 to about nucleotide 151 of the nucleotide sequence of SEQ ID NO:82 (CKX4) (e.g., SEQ ID NO:95);
(e) about nucleotide 690 to about nucleotide 790 of the nucleotide sequence of SEQ ID NO:84 (CKX5), or about nucleotide 43 to about nucleotide 143 of the nucleotide sequence of SEQ ID NO:91 (CKX5) (e.g., SEQ ID NO:97); and/or
(f) about nucleotide 1562 to about nucleotide 1709 of the nucleotide sequence of SEQ ID NO:87 (CKX6) or about nucleotide 31 to about nucleotide 178 (CKX6) of the nucleotide sequence of SEQ ID NO:88 (CKX6) (e.g., SEQ ID NO:98).

36. The plant cell of claim 30, wherein the editing system further comprises a nuclease, and the nucleic acid binding domain binds to a target site in the CKX gene, wherein the CKX gene comprises a sequence having at least 80% sequence identity to any one of the nucleotide sequences of SEQ ID NOs:72, 73, 75, 76, 78, 79, 81, 82, 84, 87, 88, or 91, comprises a region having at least 80% sequence identity to any one of the nucleotide sequences of SEQ ID NOs:93-98, and/or encodes a polypeptide having at least 80% identity to any one of the amino acid sequences of SEQ ID NOs: 74, 77, 80, 83, 89, or 92, and the at least one non-natural mutation is made following cleavage by the nuclease.

37. The plant cell of claim 36, wherein the target site comprises a sequence having at least 80% sequence identity to any one of the nucleotide sequences of SEQ ID NOs:93-98.

38-116. (canceled)
